(19) United States
(12) Patent Application Publication
(10) Pub. No.: US 2024/0307470 A1
(43) Pub. Date: Sep. 19, 2024

(54) ARMED SENECA VALLEY VIRUS ONCOLYTIC THERAPY COMPOSITIONS AND METHODS THEREOF

(71) Applicant: Seneca Therapeutics, Inc., Blue Bell, PA (US)

(72) Inventors: Paul L. HALLENBECK, Doylestown, PA (US); Sunil CHADA, Houston, TX (US)

(21) Appl. No.: 18/272,986

(22) PCT Filed: Jan. 19, 2022
(86) PCT No.: PCT/US2022/013001

§ 371 Date: Jul. 18, 2023

Related U.S. Application Data
(60) Provisional application No. 63/138,999, filed on Jan. 19, 2021.

(51) Int. Cl. A61K 35/768; A61K 31/165; A61K 31/553; A61K 38/18; A61K 38/19; A61K 38/20; A61K 38/50; A61K 39/395; A61K 45/06; C12N 15/86

(52) U.S. Cl. CPC A61K 35/768; A61K 31/165; A61K 31/553; A61K 38/1841; A61K 38/195; A61K 38/2013; A61K 38/2086; A61K 38/50; A61K 39/3955; A61K 45/06; C12N 15/86; C12Y 305/04001; C12N 2770/32022; C12N 2770/32032; C12N 2770/32043

(57) ABSTRACT

Provided herein in are armed Seneca Valley Viruses which have been altered to carry a therapeutic payload, i.e. to encode an agent for treating cancer. These armed Seneca Valley Viruses are oncolytic and express a cancer treating agent. Also provided herein are compositions and methods of using an armed Seneca Valley Virus to treat cancer in a subject.

### CROSS-REFERENCE TO RELATED APPLICATIONS

This application claims priority to U.S. Provisional Application No. 63/138,999, filed Jan. 19, 2021, the disclosure of which is incorporated by reference in its entirety.

### SEQUENCE LISTING

The instant application contains a Sequence Listing which has been submitted electronically in ASCII format and is hereby incorporated by reference in its entirety. Said ASCII copy, created on Jan. 19, 2022, is named 115029_000049_SL.txt and is 142,398 bytes in size.

### TECHNICAL FIELD

The disclosed inventions relate to compositions and methods for treating cancer. More particularly, the disclosed inventions relate to the field of treating cancer in a subject using an oncolytic virus, in particular Seneca Valley Virus, which has been engineered to encode a therapeutic agent that helps in the treatment of cancer.

### BACKGROUND

Cancer is the second most common cause of death in the United States. One out of every four individuals dies from it, and more than one million new cancer diagnoses are made every year. The disease begins with the uncontrolled proliferation and growth of abnormal, transformed cells. However, the definition does not end with a description of one disease but of hundreds of different diseases. No two cancers are the same, nor are they clonal. The mutations driving and bought during cell transformation may be similar, but they are never identical. This conundrum adds to the complexity and heterogeneity of the pathologies that patients develop. Current cancer therapies, including chemotherapeutics and radiation, are most effective when combined with immunomodulatory agents to create and enhance the antitumor microenvironment. Many malignancies may be resistant to treatment via these traditional methods.

Oncolytic viruses show enormous potential as anti-cancer agents. The picornavirus Seneca Valley virus (SVV) is a single stranded (+) RNA virus that has been investigated as an oncolytic therapy. It has been shown that SVV can target and facilitate regression of many intractable malignancies, including small and non-small cell lung cancers and pediatric solid tumors. Often, these oncolytic viruses are used in conjunction with another compound useful for treating cancer. Such combination therapy may require the administration of different compound via different routes.

Accordingly, what is needed is an improved therapeutic approach for using oncolytic viruses, in particular SVV, in combination with another agent useful for treating cancer.

### SUMMARY

Provided herein are armed Seneca Valley Viruses, which have been genetically engineered that to express an agent that is useful to treat cancer. In certain embodiments, the agent is a binding fragment of an anti-PD-1 antibody, a CXCL9, a TGFβ receptor decoy, an IL-2 mutant, or a nitroreductase. In certain embodiment, the disclosure provides an armed Seneca Valley Virus that comprises Seneca Valley Virus or oncolytic fragment thereof and a nucleic acid encoding a therapeutic protein of interest such as an interleukin, a chemokine, or a nanobody acting as a checkpoint inhibitor. In certain embodiments, the armed Seneca Valley Virus comprises Seneca Valley Virus or oncolytic fragment thereof into which the nucleic acid encoding a therapeutic protein of interest has been inserted.

The armed Seneca Valley Virus may comprise the sequence of a Seneca Valley Virus or oncolytic fragment thereof into which a nucleic acid at least 85%, 95%, 99%, or 100% identical to the nucleic acid of SEQ ID NO: 1, 3, 5, 7, 9, 11, 29, 31, 33, 35, 37, 39, 41, 43, 45, 47, 49, or 51 has been inserted. Alternatively, the armed Seneca Valley Virus comprises the sequence of a Seneca Valley Virus or oncolytic fragment thereof into which a nucleic acid encoding a protein at least 85%, at least 90%, at least 95%, at least 99%, or 100% identical to the amino acid sequence of SEQ ID NO: 2, 4, 6, 8, 10, 12, 30, 32, 34, 36, 38, 40, 42, 44, 46, 48, 50, or 52 has been inserted.

Alternatively, the armed Seneca Valley Viruses comprises a nucleotide sequence at least 85%, at least 90%, at least 95%, at least 99%, or 100% identical to: nucleotides 1-7762 of SEQ ID NO: 13; nucleotides 1-7783 of SEQ ID NO: 14; nucleotides 1-7759 of SEQ ID NO: 15; nucleotides 1-7984 of SEQ ID NO: 16; nucleotides 1-8140 of SEQ ID NO: 17; or nucleotides 1-7738 of SEQ ID NO: 18.

The disclosure also provides for vectors such as plasmids containing an armed Seneca Valley Virus. In certain embodiments, plasmid comprises SEQ ID NO: 13-18 or 53-64 or a fragment thereof.

Also provided herein are improved methods, compositions, kits, and pharmaceutical composition for treating cancer which use an armed Seneca Valley Virus, which has been genetically engineered to express an agent that is useful to treat cancer.

In particular, the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer. The cancer may also be a neuroblastoma, a melanoma, a neuroendocrine cancer, or a small cell lung cancer (SCLC) tumor.

One embodiment of the invention is a method of treating a cancer in a subject in need thereof comprising administering to the subject an effective amount of an armed Seneca Valley Virus, wherein the virus has been genetically engineered to express an agent useful to treat cancer.

Another embodiment of the invention is a method of improving the success of oncolytic cancer virus treatment comprising administering an effective amount of an armed Seneca Valley Virus, wherein the virus has been genetically engineered to express an agent useful to treat cancer.

Also provided herein is a pharmaceutical composition for treating a cancer in a subject, the pharmaceutical composition comprising an armed SVV, and a pharmaceutical acceptable carrier, wherein the armed SVV has been genetically engineered to express an agent useful to treat cancer.

Further provided herein is a kit for treating cancer in a subject comprising an armed Seneca Valley Virus, wherein the armed SVV has been altered to express an agent useful to treat cancer.

Additionally, provided herein is an armed Seneca Valley Virus (SVV) for use in the manufacture of a medicament for treatment of a cancer, wherein the armed SVV encodes an agent useful to treat cancer.

Another embodiment of the invention is an armed SVV. Yet another embodiment is a method of generating an armed SVV. The armed SVV has been altered to carry a nucleic acid encoding an agent useful for treating cancer.

Other features and advantages of the invention will be apparent from the detailed description and examples that follow.

### BRIEF DESCRIPTION OF THE DRAWINGS

The summary, as well as the following detailed description, is further understood when read in conjunction with the appended drawings. For the purpose of illustrating the invention, there are shown in the drawings' exemplary embodiments of the invention. However, the invention is not limited to the specific methods and compositions disclosed and the invention is not limited to the precise arrangements and instrumentalities of the embodiments depicted in the drawings. In addition, the drawings are not necessarily drawn to scale. In the drawings:

FIG. 1 shows a map of plasmid pNTX-11 VHH aPDL-1 (SEQ ID NO: 13).

FIG. 2 shows a schematic of how to generate the plasmid pNTX-11 VHH aPDL-1 (SEQ ID NO: 13).

FIG. 3 shows a map of plasmid pNTX-11 IL2 quad mutant (SEQ ID NO: 14).

FIG. 4 shows a schematic of how to generate the plasmid pNTX-11 IL2 quad mutant (SEQ ID NO: 14).

FIG. 5 shows a map of plasmid pNTX-11 CXCL9 (SEQ ID NO: 15).

FIG. 6 shows a schematic of how to generate the plasmid pNTX-11 CXCL9 (SEQ ID NO: 15).

FIG. 7 shows a map of plasmid pNTX-11+ TGFbDNRII (SEQ ID NO: 16).

FIG. 8 shows a schematic of how to generate the plasmid pNTX-11+TGFbDNRII (SEQ ID NO: 16).

FIG. 9 shows a map of plasmid pNTX-11 nfsa mut 22 (SEQ ID NO: 17).

FIG. 10 shows a schematic of how to generate the plasmid pNTX-11 nfsa mut 22 (SEQ ID NO: 17).

FIG. 11 shows a map of plasmid pNTX-11 Neoleukin 2-15 (SEQ ID NO: 18).

FIG. 12 shows a schematic of how to generate the plasmid pNTX-11 Neoleukin 2-15 (SEQ ID NO: 18).

FIG. 13 shows a map of plasmid pNTX-11 ova+covid epitopes (SEQ ID NO: 19).

FIG. 14 shows a schematic of how to generate the plasmid pNTX-11 ova+covid epitopes (SEQ ID NO: 19).

FIG. 15A shows a schematic of the Seneca Valley Virus (SVV-001) genome. Specifically, FIG. 15A shows the insertion site for the armed constructs of the invention between the nucleotide sequences encoding SVV protein 2A and SVV protein 2B.

FIG. 15B shows a closeup schematic view of the insertion site including restriction enzyme binding sites.

FIG. 15C shows the strategy for insertion of exogenous GFP-coding sequences into the SVV-001 genome. A portion of the SVV-001 polyprotein is reproduced with gaps of long sequence between junctions indicated by dashes. Proteolytic events are indicated by arrowheads, whilst ribosome skips are indicated by diamond heads.

FIG. 16A and FIG. 16B show the rapid generation of armed SVV viruses in cell lines. FIG. 16A shows the results for SVV-GFP (SVV engineered to express GFP and FIG. 16B shows the results for SVV-mCherry (SVV engineered to express mCherry).

FIG. 17 shows the RT-PCR data for armed SVV generated to express IL-2, CXCL9, and IL-2/15. The RT-PCR data demonstrates that the armed SVV express therapeutic transgenes.

FIG. 18 shows the transfection of linearized DNA in PerC-T7 pol cells.

FIG. 19A and FIG. 19B show the IL-2 and IL2-15 bioassay.

FIG. 20A and FIG. 20B show the functional activity of SVV-IL2 and SVV-IL2/15 (Neoleukin 2-15). FIG. 20A shows the activity for SVV-IL2 and SVV-IL2/15 (Neoleukin 2-15). FIG. 20B shows the activity for the IL-2 standard positive control.

FIG. 21A and FIG. 21B show the data for SVV-CXCL9 Elisa. FIG. 21A shows the human CXCL standard curve. FIG. 21B shows the human CXCL9 level detection in amplified SVV-CXCL9 supernatants.

FIG. 22A shows the nucleic acid sequence of plasmid pSVV-aCTLA4 VHH (SEQ ID NO: 53) containing the armed SVV encoding the anti-CLA nanobody of SEQ ID NO: 30.

FIG. 22B shows the map of plasmid pSVV-aCTLA4 VHH.

FIG. 23A shows the nucleic acid sequence of plasmid pSVV-CD3 VHH (SEQ ID NO: 54) containing the armed SVV encoding the anti-CD3 nanobody of SEQ ID NO: 32.

FIG. 23B shows the map of plasmid pSVV-CD3 VHH.

FIG. 24A shows the nucleic acid sequence of plasmid pSVV-new aPDL1 VHH v. 2 (SEQ ID NO: 55) containing the armed SVV encoding the anti-PDL1 nanobody of SEQ ID NO: 34.

FIG. 24B shows the map of plasmid pSVV-new aPDL1 VHH v. 2.

FIG. 25A shows the nucleic acid sequence of plasmid pSVV-new aPDL1-GSS-aCTLA4 (SEQ ID NO: 56) containing the armed SVV encoding the anti-PDL1 and anti-CTLA4 nanobodies of SEQ ID NO: 36.

FIG. 25B shows the map of plasmid pSVV-new aPDL1-GSS-aCTLA4.

FIG. 26A shows the nucleic acid sequence of plasmid pSVV-aCD3-GSS-aCTLA4 (SEQ ID NO: 57) containing the armed SVV encoding the anti-CD3 and anti CTLA4 nanobodies of SEQ ID NO: 38.

FIG. 26B shows the map of plasmid pSVV-aCD3-GSS-aCTLA4.

FIG. 27A shows the nucleic acid sequence of plasmid pSVV-new aPDL1-GSS-aCD3 (SEQ ID NO: 58) containing the armed SVV encoding the anti-CD3 and anti-PDL1 nanobodies of SEQ ID NO: 40.

FIG. 27B shows the map of plasmid pSVV-new aPDL1-GSS-aCD3.

FIG. 28A shows the nucleic acid sequence of plasmid pSVV-hIL-2 v.3 no signal seq (SEQ ID NO: 59) containing the armed SVV encoding IL-2 of SEQ ID NO: 42.

FIG. 28B shows the map of plasmid pSVV-hIL-2 v.3 no signal seq.

FIG. 29A shows the nucleic acid sequence of plasmid pSVV-hIL-2 v.2 (SEQ ID NO: 60) containing the armed SVV encoding IL-2 of SEQ ID NO: 44.

FIG. 29B shows the map of plasmid pSVV-hIL-2 v.2.

FIG. 30A shows the nucleic acid sequence of plasmid pSVV-TGFbR DN v.2 no signal seq+met+pro (SEQ ID NO: 61) containing the armed SVV encoding the TGF-beta RII decoy of SEQ ID NO: 46.

FIG. 30B shows the map of pSVV-TGFbR DN v.2 no signal seq+met+pro.

FIG. 31A shows the nucleic acid sequence of plasmid pSVV-TGFbR DN delta v.3 ser+met (SEQ ID NO: 62) containing the armed SVV encoding the TGF-beta RII decoy of SEQ ID NO: 48.

FIG. 31B shows the map of plasmid pSVV-TGFbR DN delta v.3 ser+met.

FIG. 32A shows the nucleic acid sequence of plasmid pSVV-FCY2+3 mutations (SEQ ID NO: 63) encoding the cytosine deaminase of SEQ ID NO: 50.

FIG. 32B shows the map of plasmid pSVV-FCY2+3 mutations.

FIG. 33A shows the nucleic acid sequence of plasmid pSVV-nfsa mut 22-78 encoding the Nfsa mutant of SEQ ID NO: 52.

FIG. 33B shows the map of plasmid pSVV-nfsa mut 22-78.

### DETAILED DESCRIPTION OF THE INVENTION

The general description and the following detailed description are exemplary and explanatory only and are not restrictive of the invention, as defined in the appended claims. Other aspects of the present invention will be apparent to those skilled in the art in view of the detailed description of the invention as provided herein.

The invention relates to an armed Seneca Valley Virus (SVV), which encodes an agent useful for treating cancer. The invention also relates to methods of generating such an armed SVV.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein the term “Seneca Valley Virus” or “SVV” encompass wild type SVV or an SVV derivative. Exemplary suitable SSV strains include SVV-001, NTX-010, and the SVV strain having ATCC Patent Deposit Number PTA-5343. As used herein, the term “derivative” specifies that a derivative of a virus can have a nucleic acid or amino acid sequence difference in respect to a template viral nucleic acid or amino acid sequence. As used herein, the term SVV also encompasses fragments of SVV that maintain the oncolytic activity of SVV. For instance, an SVV derivative can refer to an SVV that has a nucleic acid or amino acid sequence different with respect to the wild-type SVV nucleic acid or amino acid sequence of ATCC Patent Deposit Number PTA-5343. In other embodiments, the SVV derivative may be the ONCR-788. In some embodiments, the SVV derivative encompasses an SVV mutant, an SVV variant or a modified SVV (e.g. genetically engineered SVV). In some embodiments, the modified SVV derivative is modified to be capable of recognizing different cell receptors or to be capable of evading the immune system while still being able to invade, replicate and kill the cell of interest (i.e. cancer cell). In general, an SVV or SVV derivative can be derived from an already pre-existing stock of virus that is passaged to produce more viruses. SVV or SVV derivative can also be derived from a plasmid,

As used herein, the phrases “armed Seneca Valley Virus” or “armed SVV” encompass a “Seneca Valley Virus” or “SVV” as defined above, that has been modified to express an agent that is useful for treating cancer. An armed SVV encodes an agent useful for treating cancer. An armed SVV has been engineered to express therapeutic genes.

As used herein, “higher” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control reference. A disclosed herein an expression level higher than a reference value refers to an expression level (mRNA or protein) that is higher than a normal or control level from an expression (mRNA or protein) measured in a healthy subject or defined or used in the art.

As used herein, “lower” refers to expression levels which are at least 10% lower or more, for example, 20%, 30%, 40%, or 50%, 60%, 70%, 80%, 90% lower or more, and/or 1.1 fold, 1.2 fold, 1.4 fold, 1.6 fold, 1.8 fold, 2.0 fold lower or more, and any and all whole or partial increments in between, than a control reference. A disclosed herein an expression level lower than a reference value refers to an expression level (miRNA or protein) that is lower than a normal or control level from an expression (mRNA or protein) measured in a healthy subject or defined or used in the art.

As used herein, the terms “control,” or “reference” can be used interchangeably and refer to a value that is used as a standard of comparison.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime. For purposes herein, a combination therapy can include a treatment regime that includes administration of an oncolytic virus and another anti-cancer agent, each for treating the same hyperproliferative disease or conditions, such as the same tumor or cancer. Combination therapy can also include using an armed SVV in combination with an unarmed SVV.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. As used herein, when a nucleic acid sequenced is provided as a DNA sequence, it should be understood that the RNA sequence may also be used.

Nucleic acids can be single stranded or double-stranded or can contain portions of both double-stranded and single-stranded sequence. The nucleic acid can be DNA, both genomic and cDNA, RNA, or a hybrid, where the nucleic acid can contain combinations of deoxyriboand ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. “Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and CG) or Hoogsteen base pairing between nucleotides or nucleotide analogs of nucleic acid molecules.

Consensus” or “Consensus Sequence” as used herein may mean a synthetic nucleic acid sequence, or corresponding polypeptide sequence, constructed based on analysis of an alignment of multiple subtypes of a particular antigen. The sequence may be used to induce broad immunity against multiple subtypes, serotypes, or strains of a particular antigen. Synthetic antigens, such as fusion proteins, may be manipulated to generate consensus sequences (or consensus antigens).

“Fragment” as used herein means a nucleic acid sequence or a portion thereof that encodes an armed SVV which is able to be oncolytic and in which the encodes a protein capable of treating cancer.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. Identity can be performed manually or by using a computer sequence algorithm such as BLAST or BLAST 2.0.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein means a nucleic acid sequence containing an origin of replication. A vector can be a viral vector, bacteriophage, bacterial artificial chromosome, or yeast artificial chromosome. A vector can be a DNA or RNA vector. A vector can be a self-replicating extrachromosomal vector, and preferably, is a DNA plasmid.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to intra-tumoral, intravenous, intrapleural, oral, aerosol, parenteral, ophthalmic, pulmonary, and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) (e.g. an armed SVV) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the virus particle or infectious units generated from vector of the invention which is required to prevent the particular disease condition, or which reduces the severity of and/or ameliorates the disease condition or at least one symptom thereof or condition associated therewith.

As used herein the phrase “cancer refractory to monotherapy” with the checkpoint inhibitor refers to any cancer that may be resistant at the beginning of treatment to monotherapy with a checkpoint inhibitor, or becomes resistant to monotherapy with a checkpoint inhibitor during treatment. The phrase includes cancers that have been treated with the checkpoint inhibitors but have not responded (i.e. are resistant to the cancer treatment). The phrase also includes cancers that have been treated with a checkpoint inhibitor and initially responded to the treatment, but subsequently the tumor regrows (relapsed/resistant). For the proposes of this definition, the term monotherapy with a checkpoint inhibitor refers cancer that have been treated with a checkpoint inhibitor as the only anti-cancer agent. Examples of such cancers include cold tumors, which are cancers that have not been recognized or have not provoked a strong response by the immune system.

Cold tumors are resistant to checkpoint inhibitors and/or checkpoint blockage.

A “subject” or “patient,” as used therein, may be a human or non-human mammal. Non-human mammals include, for example, livestock and pets, such as ovine, bovine, porcine, canine, feline and murine mammals. Preferably, the subject is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Armed SVV

The disclosure provides for armed Seneca Valley Viruses which have been altered to carry a therapeutic payload, i.e. to encode an agent for treating cancer. Without being bound by theory, it is contemplated that when such armed SVV are used to treat cancer to success of cancer treatment is improved compared to treatment using Seneca Valley Virus that has not been armed (i.e. an SVV that is not carrying a therapeutic payload).

In certain embodiments, the disclosure provides for a Seneca Valley Virus which has been altered to encode a binding fragment of an anti-PD-1 antibody, CXCL9, a TGFβ receptor decoy, an IL-2 mutant, or a nitroreductase.

Additional embodiments of the disclosure include: armed SVV encoding anti-CTLA4 nanobody; armed SVV encoding an anti-CD3 nanobody; armed SVV encoding an anti-PDL1 nanobody; armed SVV encoding both an anti-CTLA4+anti-PDL1 nanobody; armed SVV encoding both an anti-CTLA4+anti-CD3 nanobody; armed SVV encoding both an anti-CD3+anti-PDL1 nanobody; armed SVV encoding IL-2 (versions 2 and 3); armed SVV encoding TGF-beta dominant negative RII decoy-no SS v.2; armed SVV encoding TGF-beta dominant negative RII decoy; armed SVV encoding cytosine deaminase; and armed SVV encoding Nfsa mut 22-78.

In one embodiment, the disclosure provides for an armed Seneca Valley Virus contains a nucleic acid encoding an IL-2 quadruple mutant such as IL-2 quadruple mutant T3A/F42A/Y45A/L72G (C125A). In another embodiment, the disclosure provides for an armed SVV which contains neoleukin 2-15.

In certain embodiments, the disclosure is directed to an armed Seneca Valley Virus, wherein the armed Seneca Valley Virus comprises Seneca Valley Virus or oncolytic fragment thereof and a nucleic acid encoding a therapeutic protein of interest. In certain embodiments, the protein of interest is an interleukin, a chemokine, or a nanobody acting as a checkpoint inhibitor. For example, the protein of interest may be an anti-PD-L1 nanobody, IL-2, CXCL9, IL-15, IL-2/IL-15, a TGF-β decoy, NfsA. In other embodiments, the protein of interest comprises an interleukin, a chemokine, or a nanobody acting as a checkpoint inhibitor. In some embodiments, the therapeutic protein of interest includes an anti-PD-L1 nanobody, IL-2 or mutant thereof, CXCL9, IL-15, IL-2/IL-15 (Neoleukin 2-15), a TGF-β decoy or mutant thereof, NfsA or mutant thereof, an anti-CTLA4 nanobody, an anti-CD3 nanobody, an anti-CTLA-4+anti-PDLI-1 nanobody, an anti-CLTA4+anti-PLD-1 nanobody, or a cytosine deaminase.

In certain embodiments, the armed Seneca Valley Virus comprises Seneca Valley Virus or a fragment thereof into which the nucleic acid encoding a therapeutic protein of interest has been inserted.

In certain embodiments, the armed Seneca Valley Virus is generated by inserting a nucleic acid sequence encoding a therapeutic protein into the genome of a Seneca Valley Virus or oncolytic fragment thereof between the coding sequences for protein 2A and 2B.

TGF-β decoy receptors bind to TGF-β (e.g. TGF-β1, TGF-β2 and/or TGF-β3) and are derived from TGF-beta receptors lacking the amino acid sequence encoding a transmembrane domain. Expression of TGF-b decoys will reduce the immunosuppressive milieu in the tumor microenvironment and augment T cell responses.

Neoleukin 2-15, is an improved IL-2 mutant which lacks binding site for IL-2Rα (also called CD25) or IL-15Rα (also known as CD215). The molecule is hyper-stable, binds human and mouse IL-2Rβγc with higher affinity than the natural cytokines. The molecule is hyper-stable, binds human and mouse IL-2Rβγc with higher affinity than the natural cytokines.

In certain embodiments, the SVV is armed with an IL-2 mutein that is capable of only binding to CD122 and CD132 (beta and gamma chains) of IL-2 receptor and lacks a CD25 binding domain.

Certain embodiments of the invention are directed to an armed Seneca Valley Virus, which is generated by inserting a nucleic acid of SEQ ID NO: 1, 3, 5, 7, 9, 11, 29, 31, 33, 35, 37, 39, 41, 43, 45, 47, 49, or 51 into a Seneca Valley Virus, wherein the resulting armed Seneca Valley Virus is oncolytic and wherein the therapeutic protein encoded by SEQ ID NO: 1, 3, 5, 7, 9, 11, 29, 31, 33, 35, 37, 39, 41, 43, 45, 47, 49, or 51 is expressed when the Seneca Valley Virus is administered to a cancer cell.

Certain embodiments of the invention are directed to an armed Seneca Valley Virus, which is generated by inserting a nucleic acid at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1, 3, 5, 7, 9, 11, 29, 31, 33, 35, 37, 39, 41, 43, 45, 47, 49, or 51 into a Seneca Valley Virus, wherein the resulting armed Seneca Valley Virus is oncolytic and wherein the therapeutic protein encoded by SEQ ID NO: 1, 3, 5, 7, 9, 11, 29, 31, 33, 35, 37, 39, 41, 43, 45, 47, 49, or 51 is expressed when the Seneca Valley Virus is administered to a cancer cell.

Another embodiment of the invention is an armed Seneca Valley Virus, which contains the sequence of a Seneca Valley Virus or oncolytic fragment thereof that has been altered to express a protein of SEQ ID NO: 2, 4, 6, 8, 10, 12, 30, 32, 34, 36, 38, 40, 42, 44, 46, 48, 50, or 52, wherein the armed Seneca Valley Virus is oncolytic and wherein the therapeutic protein is expressed when the Seneca Valley Virus is administered to a cancer cell.

Yet another embodiment of the invention is an armed Seneca Valley Virus, which contains the sequence of a Seneca Valley Virus or oncolytic fragment thereof that has been altered to express a protein at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the protein sequence of SEQ ID NO: 2, 4, 6, 8, 10, 12, 30, 32, 34, 36, 38, 40, 42, 44, 46, 48, 50, or 52, wherein the armed Seneca Valley Virus is oncolytic and wherein the therapeutic protein is expressed when the Seneca Valley Virus is administered to a cancer cell.

An alternate embodiment of the invention is an armed Seneca Valley Virus, which contains the sequence of a Seneca Valley Virus or oncolytic fragment thereof that has been altered to include a nucleic acid sequence encoding the protein of SEQ ID NO: 2, 4, 6, 8, 10, 12, 30, 32, 34, 36, 38, 40, 42, 44, 46, 48, 50, or 52, wherein the armed Seneca Valley Virus is oncolytic and wherein the therapeutic protein is expressed when the Seneca Valley Virus is administered to a cancer cell.

Yet another embodiment of the invention is an armed Seneca Valley Virus, which contains the sequence of a Seneca Valley Virus or oncolytic fragment thereof that has been altered to include a nucleic acid sequence encoding a protein at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the protein sequence of SEQ ID NO: 2, 4, 6, 8, 10, 12, 30, 32, 34, 36, 38, 40, 42, 44, 46, 48, 50, or 52, wherein the armed Seneca Valley Virus is oncolytic and wherein the therapeutic protein is expressed when the Seneca Valley Virus is administered to a cancer cell.

In one embodiment, the armed Seneca Valley Virus comprises nucleotides 1-7762 of SEQ ID NO: 13. In another embodiment, the armed Seneca Valley Virus comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7762 of SEQ ID NO: 13.

In another embodiment, the armed Seneca Valley Virus comprises nucleotides 1-7783 of SEQ ID NO: 14. In another embodiment, the armed Seneca Valley Virus comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7783 of SEQ ID NO: 14.

In an alternate embodiment, the armed Seneca Valley Virus comprises nucleotides 1-7759 of SEQ ID NO: 15. In another embodiment, the armed Seneca Valley Virus comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7759 of SEQ ID NO: 15.

In yet another alternate embodiment, the armed Seneca Valley Virus comprises nucleotides 1-7984 of SEQ ID NO: 16. In another embodiment, the armed Seneca Valley Virus comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7984 of SEQ ID NO: 16.

In yet an alternate embodiment, the armed Seneca Valley Virus comprises nucleotides 1-8140 of SEQ ID NO: 17. In another embodiment, the armed Seneca Valley Virus comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-8140 of SEQ ID NO: 17.

In an additional embodiment, the armed Seneca Valley Virus comprises nucleotides 1-7738 of SEQ ID NO: 18. In another embodiment, the armed Seneca Valley Virus comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7738 of SEQ ID NO: 18.

Another embodiment of the invention is an armed Seneca Valley Virus generated by inserting a nucleic acid sequence encoding a therapeutic protein into the genome of a Seneca Valley Virus between the coding sequences for protein 2A and 2B. The nucleic acid encoding the therapeutic protein may be comprise: a nucleic acid of SEQ ID NO: 1, 3, 5, 7, 9, 11, 29, 31, 33, 35, 37, 39, 41, 43, 45, 47, 49, or 51; a nucleic acid at least 85%, 95%, or 99% identical to the nucleic acid of SEQ ID NO: 1, 3, 5, 7, 9, 11, 29, 31, 33, 35, 37, 39, 41, 43, 45, 47, 49, or 51; a nucleic acid encoding the amino acid sequence of SEQ ID NO: 2, 4, 6, 8, 10, 12, 30, 32, 34, 36, 38, 40, 42, 44, 46, 48, 50, or 52; or a nucleic acid encoding a protein at least 85%, at least 90%, at least 95%, or at 99% identical to the amino acid sequence of SEQ ID NO: 2, 4, 6, 8, 10, 12, 30, 32, 34, 36, 38, 40, 42, 44, 46, 48, 50, or 52. In certain embodiments, the armed Seneca Valley Virus is generated by arming SVV-001.

In another embodiment, the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted. In yet another embodiment, the armed Seneca Valley Virus comprises the sequence of a Seneca Valley Virus or oncolytic fragment thereof into which a nucleic acid at least at least 85%, 95%, or 99% identical to nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 has been inserted. The nucleic acids may be inserted between the coding sequences for protein 2A and 2B in SVV or SVV derivative.

### Vectors Carrying Armed SSV

The disclosure also provides for vectors containing the armed SVV constructs of the disclosure. The vector can have a nucleic acid sequence containing an origin of replication. The vector can be a plasmid, bacteriophage, bacterial artificial chromosome, or yeast artificial chromosome. The vector can be either a self-replicating extrachromosomal vector or a vector which integrates into a host genome.

In one embodiment, the vector comprises a nucleic acid sequence at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 13-18 or 53-64.

In certain embodiments, the vector is a plasmid forming the nucleic acid sequence of any one of SEQ ID NO: 13-18 or 53-64 or a nucleic acid substantially identical to the nucleic acid sequence of SEQ ID NO: 13-18 or 53-64.

In one embodiment, the plasmid comprises nucleotides 1-7762 of SEQ ID NO: 13. In another embodiment, the plasmid comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7762 of SEQ ID NO: 13.

In another embodiment, the plasmid comprises nucleotides 1-7783 of SEQ ID NO: 14. In another embodiment, the plasmid comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7783 of SEQ ID NO: 14.

In an alternate embodiment, the plasmid comprises nucleotides 1-7759 of SEQ ID NO: 15. In another embodiment, the plasmid comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7759 of SEQ ID NO: 15.

In yet another alternate embodiment, the plasmid comprises nucleotides 1-7984 of SEQ ID NO: 16. In another embodiment, the plasmid comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7984 of SEQ ID NO: 16.

In yet an alternate embodiment, the plasmid comprises nucleotides 1-8140 of SEQ ID NO: 17. In another embodiment, the plasmid comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-8140 of SEQ ID NO: 17.

In an additional embodiment, the plasmid comprises nucleotides 1-7738 of SEQ ID NO: 18. In another embodiment, the plasmid comprises a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7738 of SEQ ID NO: 18.

The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins. The vectors may have expression signals such as a strong promoter, a strong termination codon, adjustment of the distance between the promoter and the cloned gene, and the insertion of a transcription termination sequence and a PTIS (portable translation initiation sequence).

### Methods of Arming SVV

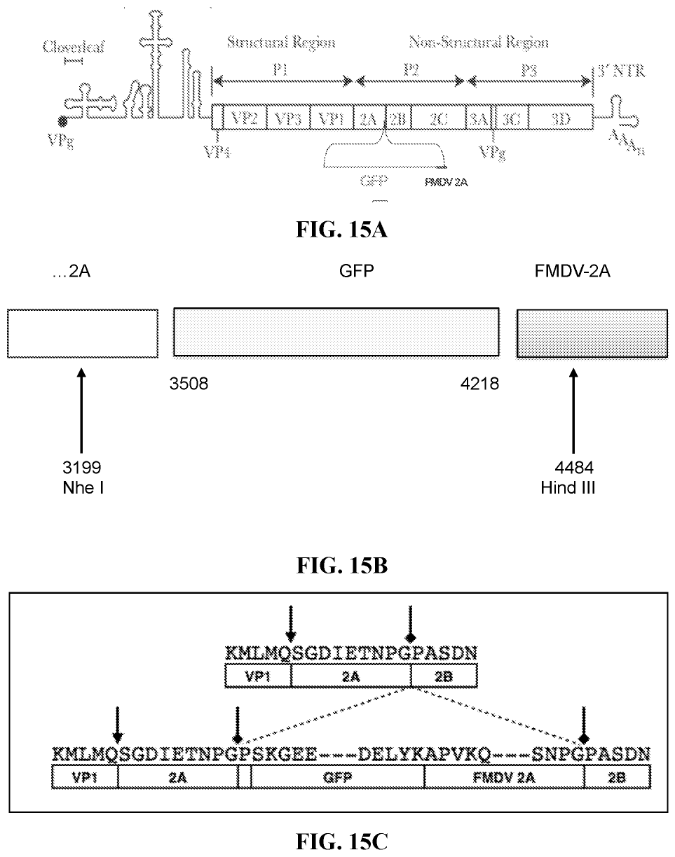

The disclosure also provides for methods of arming a Seneca Valley Virus to express a therapeutic protein. The methods include providing a Seneca Valley Virus nucleic acid sequence such as nucleic acid sequence of SVV-001, NTX-010, and the SVV strain having ATCC Patent Deposit Number PTA-5343 and then inserting the therapeutic protein at the appropriate location, whereby the resultant armed SSV virus is oncolytic and expresses the therapeutic protein. In certain embodiments, the therapeutic protein is inserted into the Seneca Valley Virus between the nucleic sequence encoding the 2A peptide and the nucleic acid sequence encoding the 2B peptide of SVV. A schematic of this insertion site is shown in FIG. 15A-C.

In certain embodiments, the methods of invention can be used to arm SVV to contain a nucleic acid encoding a therapeutic protein that is up to 800 base pairs in length. In one embodiment of the method, the Seneca Valley Virus is NTX-010 or SVV-001.

In one embodiment, the disclosure provides for methods of generating armed SVV constructs which includes following steps: (1) construct armed SVV plasmid; (2) linearize armed SVV plasmid to define 3′ end; (3) in vitro transcription reaction using T7 polymerase to generate RNA transcript with authentic 5′ and 3′ termini; (4) transfect RNA into target cells; and (5) isolate armed SVV virus.

In another embodiment, the disclosure provides for methods of generating armed SVV constructs which includes following steps: (1) clone T7 polymerase optimized mammalian expression plasmid into target cell line; (2) linearize armed SVV plasmid; (3) transfect plasmid into T7-pol cells; and (4) isolate the armed SVV virus.

In an alternate embodiment, the method comprises: constructing a plasmid comprising the Seneca Valley Virus or oncolytic fragment thereof and the nucleic acid encoding a therapeutic protein of interest; linearization of the plasmid to define 3′ end; in vitro transcription reaction using T7 polymerase to generate RNA transcript with authentic 5′ and 3′ termini; transfection of the RNA transcript into target cells; and isolation of the armed SVV virus.

In yet another embodiment, the method comprises: cloning a T7 polymerase optimized mammalian expression plasmid into target cells; providing a linearized armed SVV plasmid comprising the Seneca Valley Virus or oncolytic fragment thereof and the nucleic acid encoding a therapeutic protein of interest; transfecting the armed SVV plasmid into the T7-pol target cells; and isolating the armed Seneca Valley Virus.

In certain embodiments, the armed SVV constructs may be generated using the methods shown in the Examples below.

In certain embodiments of the invention, the method comprises inserting the nucleic acid of SEQ ID NO: 1, 3, 5, 7, 9, 11, 29, 31, 33, 35, 37, 39, 41, 43, 45, 47, 49, or 51 into a Seneca Valley Virus. In other embodiments, the method comprises inserting a nucleic acid at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1, 3, 5, 7, 9, 11, 29, 31, 33, 35, 37, 39, 41, 43, 45, 47, 49, or 51 into a Seneca Valley Virus or oncolytic fragment thereof Alternatively, the method comprises inserting a nucleic acid encoding a protein of SEQ ID NO: 2, 4, 6, 8, 10, 12, 30, 32, 34, 36, 38, 40, 42, 44, 46, 48, 50, or 52 into a Seneca Valley Virus. The method may also comprise inserting a nucleic acid encoding a protein at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the protein sequence of SEQ ID NO: 2, 4, 6, 8, 10, 12, 30, 32, 34, 36, 38, 40, 42, 44, 46, 48, 50, or 52. The methods result in the production of an armed SSV that is oncolytic and which produces a functional therapeutic protein or functional fragment thereof, i.e. the therapeutic protein or fragment thereof maintain their therapeutic functionality.

### Immunogenic SVV Construct

The disclosure also provides for a Seneca Valley Virus that has been modified to a contain a protein for screening such as for example ovalbumin and a COVID epitope Accordingly, one embodiment is a Seneca Valley Virus that comprises nucleic acids 1-7891 of SEQ ID NO: 19. Another embodiment of the invention is a Seneca Valley virus that comprises a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 1-7891 of SEQ ID NO: 19.

Yet another embodiment is an SVV construct which is generated by inserting a nucleic acid sequence into the genome of a Seneca Valley Virus between the coding sequences for protein 2A and 2B, wherein the nucleic acid sequence comprises nucleotides 3508-4014 of SEQ ID NO: 19 or wherein the nucleic acid sequences comprises a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to nucleotides 3508-4014 of SEQ ID NO: 19.

In certain embodiments, the SVV construct may be used to assess the in vivo immunogenicity of Seneca Valley Virus and/or an armed Seneca Valley Virus. Specifically, the SVV construct may be used to test the immune activating properties of SVV.

Methods of Treating Cancer with Armed SVV

The disclosure provides for methods, compositions, kits, and pharmaceutical composition for treating cancer which utilize an armed Seneca Valley Virus, whereby the armed Seneca Valley Virus encodes an agent useful for treating cancer. In particular, the disclosure provides for methods, compositions, kits, and pharmaceutical composition for treating cancer which utilize an armed Seneca Valley Virus as described herein.

The treatment of cancer provided herein may include the treatment of solid tumors or the treatment of metastasis. Metastasis is a form of cancer wherein the transformed or malignant cells are traveling and spreading the cancer from one site to another. Such cancers include cancers of the skin, breast, brain, cervix, testes, etc. More particularly, cancers may include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, pheochromocytoma, paraganglioma, meningioma, adrenocortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Kaposi's sarcoma, moles dysplastic nevi, lipoma, angioma, dermatofibroma, keloids, rhabdomyosarcoma, medulloblastoma, and psoriasis.

In some embodiments, the cancer treated by the presently disclosed methods comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

In other embodiments, the cancer is a neuroblastoma or a melanoma. In yet another embodiment, the cancer is a neuroendocrine cancer or a small cell lung cancer (SCLC) tumor.

### Combination Therapy

The compositions and methods for treating a cancer in a subject using an armed SVV described herein may be combined with at least one additional compound useful for treating cancer. The additional compound may comprise a commercially available compound, known to treat, prevent, or reduce the symptoms of cancer and/or metastasis.

In one aspect, the pharmaceutical composition disclosed herein comprises an armed SVV and a pharmaceutical acceptable carrier. The composition may also include an additional SVV. The pharmaceutical composition may be used in combination with a therapeutic agent such as an anti-tumor agent, including but not limited to a chemotherapeutic agent, an anti-cell proliferation agent or any combination thereof For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include aclacinomycin, actinomycin, anthramycin, azaserine, bleomycins, cactinomycin, calicheamicin, carubicin, caminomycin, carzinophilin, chromomycin, dactinomycin, daunorubicin, detorubicin, 6-diazo-5-oxo-L-norleucine, doxorubicin, epirubicin, esorubicin, idarubicin, marcellomycin, mitomycins, mitoxantrone, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are used for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine.

Other examples of chemotherapeutic agents include, but are not limited to, the following and their pharmaceutically acceptable salts, acids and derivatives: MEK inhibitors, such as but not limited to, refametinib, selumetinib, trametinib or cobimetinib; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatrexate; defofamine; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K (PSK); razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and docetaxel (TAXOTERE, Rhone-Poulene Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; and capecitabine.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria toxin, mitogillin, ricin, botulinum toxin, cholera toxin, saporin 6, or a combination thereof.

An immunotherapeutic agent may be, but is not limited to, an interleukin-2 or other cytokine, an inhibitor of programmed cell death protein 1 (PD-1) signaling such as a monoclonal antibody that binds to PD-1, Ipilimumab. The immunotherapeutic agent can also block cytotoxic T lymphocytes associated antigen A-4 (CTLA-4) signaling and it can also relate to cancer vaccines and dendritic cell-based therapies.

In one embodiment the subject suffering from cancer is administered at least one anti-cancer therapeutic agent selected from the group consisting of. a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an engineered anti-cancer virus or virus derivative and a combination of any thereof.

In certain embodiments, the subject is administered a checkpoint inhibitor. The checkpoint inhibitor may be a PD-1 inhibitor, a PD-L1 inhibitor, a CTLA-4 inhibitor, or combinations thereof. In certain embodiments, the checkpoint inhibitor is an anti-PD1-antibody, an anti-PD-L1 antibody, or an anti-CTLA-4 antibody. In certain embodiment, the checkpoint inhibitor blocks one or more of the following checkpoint proteins on cancer cells: PD-1, PD-L1, CTLA-4, B7-1, B7-2. In other embodiments, the checkpoint inhibitor blocks one or more of the following checkpoint proteins: LAG-3; TIM-3; TIGIT; VISTA, B7-H3, BTLA, and Siglec-15. See Qin, S. et al. Mol Cancer 18, 155 (2019); Gaynor et al. Semin Cancer Biol. 2020 Jul. 2; S1044-579X(20)30152-8. The checkpoint inhibitor may be an antibody such as, e.g., a monoclonal antibody.

Additional exemplary suitable checkpoint inhibitors include but are not limited to ipilimumab (Yervoy®), pembrolizumab (Keytruda®), nivolumab (Opdivo®), and atezolizumab (Tecentriq®). In one embodiment, the checkpoint inhibitor is an anti-PD-1 antibody.

In one embodiment, the least one anti-cancer therapeutic agent is administered formerly, simultaneously, or subsequently to the administering of the armed SVV.

In one embodiment, the subject is administered an IFN-I inhibiting agent. The IFN-I inhibiting agent used herein encompasses any agent known in the art for inhibiting, suppressing, or reducing partially or fully and temporarily or permanently IFN type I pathway. In some embodiments, the inhibition effect of the IFN-I inhibiting agent can be reversible. In other embodiments, the inhibition of the IFN-I is reversed.

The inhibiting agent comprises siRNA, ribozyme, an antisense molecule, an aptamer, a peptidomimetic, a small molecule, an mTOR inhibitor, a histone deacetylase (HDAC) inhibitor, a Janus kinase (JAK) inhibitor, an IFN inhibitor, an IFN antibody, an IFN-a Receptor 1 antibody, an IFN-α Receptor 2 antibody and viral peptide and a combination of any thereof The viral peptide can be, but not limited to, NS1 protein from an Influenza virus or NS2B3 protease complex from dengue virus.

The mTOR pathway and its inhibition are known to be implicated in various diseases such as cancer. Rapamycin is a natural product produced by the bacterium Streptomyces hygroscopicus that can inhibit mTOR through association with its intracellular receptor FK-506 binding protein 12 (FKBP12). The FKBP12-rapamycin complex binds directly to the FKBP12-rapamycin binding domain of mTOR. mTOR functions as a catalytic subunit for two distinct molecular complexes, mTOR complex 1 (mTORC1) and mTOR complex 2 (mTORC2). mTORC1 is composed of regulatory associated protein of mTOR (Raptor) and mammalian LST8/G-protein β-subunit like protein (mLST8/GβL). This complex functions as a nutrient/energy/redox sensor and plays a role in regulating protein synthesis. The activity of mTORC1 is stimulated by insulin, growth factors, serum, phosphatidic acid, amino acids (particularly leucine) and oxidative stress (Hay and Sonenberg, Genes Dev. 18(16):1926-1945, 2004; Wullschleger et al., Cell 124(3):471-484). In contrast, mTORC1 is known to be inhibited by low nutrient levels, growth factor deprivation, reductive stress, caffeine, rapamycin, farnesylthiosalicylic acid and curcumin (Beevers et al., Int. J. Cancer 119(4):757-764, 2006; McMahon et al., Mol. Endocrinol. 19(1):175-183). The components of mTORC2 are rapamycin-insensitive companion of mTOR (Rictor), GβL, mammalian stress-activated protein kinase interacting protein 1 and mTOR. mTORC2 has been shown to function as an important regulator of the cytoskeleton through its stimulation of F-actin stress fibers, paxillin, RhoA, Rac1, Cdc42 and protein kinase C alpha (Sarbassov et al., Curr. Biol. 14(14): 1296-302, 2004; Sarbassov et al., Science 307(5712): 1098-101, 2005). Unlike mTORC1, mTORC2 is not sensitive to rapamycin.

A number of mTOR inhibitors are known in the art and have potent immunosuppressive and anti-tumor activities. Inhibitors of mTOR, such as rapamycin or rapamycin analogs or derivatives, are known to show immunosuppressive and anti-proliferative properties. Other mTOR inhibitors include everolimus, tacrolimus, zotarolimus (ABT-578), pimecrolimus, biolimus, FK-506, PP242 (2-(4-Amino-1-isopropyl-1H-pyrazolo[3,4-d]pyrimidin-3-yl)-1H-indol-5-ol), Ku-0063794 (rel-5-[2-[(2R,6S)-2,6-Dimethyl-4-morpholinyl]-4-(4-morpholinyl)pyrido[2,3-d]pyrimidin-7-yl]-2-methoxybenzenemethanol), PI-103 (3-(4-(4-Morpholinyl)pyrido[3′,2′:4,5]furo[3,2-d]pyrimidin-2-yl)phenol), PKI-179 (N-[4-[4-(4-Morpholinyl)-6-(3-oxa-8-azabicyclo[3.2.1]oct-8-yl)-1,3,5-triazin-2-yl]phenyl]-N′-4-pyridinylurea hydrochloride), AZD8055 (5-[2,4-Bis[(3S)-3-methyl-4-morpholinyl]pyrido[2,3-d]pyrimidin-7-yl]-2-methoxybenzenemethanol), WYE-132/WYE-125132 (1-{4-[1-(1,4-Dioxa-spiro[4.5]dec-8-yl)-4-(8-oxa-3-aza-bicyclo[3.2.1]oct-3-yl)-1H-pyrazolo[3,4-d]pyrimidin-6-yl]-phenyl}-3-methyl-urea), WYE-23 (4-{6-[4-(3-Cyclopropyl-ureido)-phenyl]-4-morpholin-4-yl-pyrazolo[3,4-d]pyrimidin-1-yl}-piperidine-1-carboxylic acid methyl ester), WYE-28 (4-(6-{4-[3-(4-Hydroxymethyl-phenyl)-ureido]-phenyl}-4-morpholin-4-yl-pyrazolo[3,4-d]pyrimidin-1-yl)-piperidine-1-carboxylic acid methyl ester), WYE-354 (4-[6-[4-[(Methoxycarbonyl)amino]phenyl]-4-(4-morpholinyl)-1H-pyrazolo[3,4-d]pyrimidin-1-yl]-1piperidinecarboxylic acid methyl ester), C20-methallylrapamycin and C16-(S)-butylsulfonamidorapamycin, C16-(S)-3-methylindolerapamycin (C16-iRap), C16-(S)-7-methylindolerapamycin (AP21967/C16-AiRap), CCI-779 (temsirolimus), RAD001(40-O-(2-hydroxyethyl)-rapamycin), AP-23575, AP-23675, AP-23573, 20-thiarapamycin, 15-deoxo-19-sulfoxylrapamycin, WYE-592, ILS-920, 7-epi-rapamycin, 7-thiomethyl-rapamycin, (3S,6R,7E,9R,10R,12R,14S,15E,17E,19E,21S,23S,26R,27R,34aS)-9,10,12,13,14,2-1,22,23,24,25,26,27,32,33,34,34a-Hexadecahydro-9,27-dihydroxy-3-[(1R)-2-[(-1S,3R,4R)-3-methoxy-4-tetrazol-1-yl)cyclohexyl]-1-methylethyl]-10,21-dime-t-hoxy-6,8,12,14,20,26-hexamethyl-23,27-epoxy-3H-pyrido[2,1-c] [1,4]oxaazac-yc-lohentriacontine-1,5,11,28,29(4H,6H,31H)-pentone) 23,27-Epoxy-3H pyrido [2,1-c][1,4]oxaazacyclohentriacontine-1,5,11,28,29(4H,6H,31H)-pentone (U.S. Pat. No. 6,015,815), A-94507, Deforolimus, AP-23675, AP-23841, Zotarolimus, CC1779/Temsirolimus, RAD-001/Everolimus, 7-epi-trimethoxy-rapamycin, 2-desmethyl-rapamycin, and 42-O-(2-hydroxy)ethyl-rapamycin, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, 42-O-(2-hydroxy)ethyl rapamycin, ridaforolimus, ABI-009, MK8669, TOP216, TAFA93, TORISEL™ (prodrug), CERTICAN™, Ku-0063794, PP30, Torin1, Torin2, EC0371, AP23102, AP23573, AP23464, AP23841; 40-(2-hydroxyethyl)rapamycin, 40-[3-hydroxy(hydroxymethyl) methylpropanoate]-rapamycin (also called CC1779), 32-deoxorapamycin, and 16-pentynyloxy-32(S)-dihydrorapanycin. Non-rapamycin analog mTOR inhibiting compounds include, but are not limited to, LY294002, wortmannin, quercetin, myricentin, staurosporine, and ATP competitive inhibitors. Other examples of suitable mTOR inhibitors may be found in U.S. Pat. No. 6,329,386, U.S. Publication 2003/129215, and U.S. Publication 2002/123505.

In some embodiments, the mTOR inhibitor inhibits at least one of mTORC1 and mTORC2. In other embodiments, the mTOR inhibitor is Torin 1 or Torin 2.

Many HDAC inhibitors are known and used in the art. The most common HDAC inhibitors bind to the zinc-containing catalytic domain of the HDACs. These HDAC inhibitors can be classified into several groupings named according to their chemical structure and the chemical moiety that binds to the zinc ion. Some examples include, but are not limited to, hydroxamic acids or hydroxamates (such as Trichostatin A (TSA) or Vorinostat/SAHA (FDA approved)), aminobenzamides Entinostat (MS-275), Tacedinaline (CI994), and Mocetinostat (MGCD0103), cyclic peptides (Apicidin, Romidepsin (FDA approved)), cyclic tetrapeptides or epoxyketones (such as Trapoxin B), depsipeptides, benzamides, electrophilic ketones, and carboxylic aliphatic acid compounds (such as butyrate, phenylbutyrate, valproate, and valproic acid). Other HDAC inhibitors include, but are not limited to, Belinostat (PXD101), LAQ824, and Panobinostat (LBH589). Examples of HDCA inhibitors in clinical trials include Panobinostat (LBH-589), Belinostat (PXD101), Entinostat (MS275), Mocetinostat (MGCD01030), Givinostat (ITF2357), Practinostat (SB939), Chidamide (CS055/HBI-8000), Quisinostat (JNJ-26481585), Abexinostat (PCI-24781), CHR-3996 and AR-Z2. In one embodiment, the HDAC inhibitor is Trichostatin A.

JAK inhibitors (also referred as JAK/SAT inhibitors) inhibit the activity of one or more of the Janus kinase family of enzymes (e.g. JAK1, JAK2, JAK3, and/or TYK2), thereby interfering with the JAK-STAT signaling pathway. Various JAK inhibitors are known and used in the art for the treatment of inflammatory diseases or cancer. Non-limiting examples of JAK inhibitors are FDA approved compounds including Ruxolitinib (Jakafi/Jakavi), Tofacitinib (Jakvinus, formerly known as tasocitinib and CP-690550), Oclacitinib (Apoquel), Baricitinib (Olumiant, LY3009104), Decemotinib (VX-509). Other JAK inhibitors are under clinical trials and/or used as experimental drugs. These include for instance Filgotinib (G-146034, GLPG-0634), Cerdulatinib (PRT062070), Gandotinib (LY-2784544), Lestaurtinib (CEP-701), Momelotinib (GS-0387, CYT-387), Pacritinib (SB1518), PF-04965842, Upadacitinib (ABT-494), Peficitinib (ASP015K, JNJ-54781532), Fedratinib (SAR302503), Cucurbitacin I, CHZ868, ABT-494, dimethyl fumarate (DMF, Tecfidera), GLPG0634, and CEP-33779. In one embodiment, the JAK/STAT inhibitor is staurosporine (STS; antibiotic AM-2282) which is an inhibitor of protein kinase C (PKC).

In one embodiment, the subject is further administered at least one IFN-I inhibiting agent selected from the group consisting of HDAC inhibitor, JAK/STAT inhibitor, IFN inhibitor, IFN antibody, IFN-α Receptor 1 antibody, IFN-α Receptor 2 antibody and viral peptide and a combination of any thereof. In another embodiment, the at least one IFN-I inhibiting agent is administered formerly, simultaneously, or subsequently to the administering of the armed SVV. In some embodiments, the at least one IFN-I inhibiting agent is subsequently removed once the armed SVV has replicated in the tumor cells and before the addition of an anti-cancer therapeutic agent (e.g. checkpoint inhibitor).

In one embodiment, the anti-cancer therapeutic agent is administered formerly, simultaneously, or subsequently to the administering of the at least one IFN-I inhibiting agent. In one embodiment, the anti-cancer therapeutic agent is administered subsequently to the administering of the at least one IFN-I inhibiting agent. In another embodiment, the anti-cancer therapeutic agent is administered subsequently to the administering of the at least one IFN-I inhibiting agent and the armed SVV.

In one embodiment treatment with the armed SVV is preceded by the administration of IFN-I inhibiting agent. In one embodiment, once armed SVV replication and cancer cells death are confirmed, the administration of IFN-I inhibiting agent is terminated. For instance, cancer cells can be treated with an IFN-I inhibitor, (e.g. (5-(tetradecyloxy)-2-furoic acid), acetyl-CoA carboxylase inhibitor: TOFA), 24 hours before armed SVV treatment and then both treatments can be pursued for several weeks until robust armed SVV replication is observed and markers of cell death are detected. Then the treatment with IFN-I inhibiting agent can be terminated and an anti-cancer therapeutic agent can be initiated. Upon armed SVV replication, various nucleic acids and cellular debris are generated which can trigger the activation of an influx of immune cells (e.g. T-cells, NK, cells, APCs, etc.) to proceed in cancer cells' inhibition, reduction and/or elimination/killing. This process of immune response is enhanced further by the termination of IFN-I inhibition.

### Pharmaceutical Compositions

In certain embodiments, the invention is directed to pharmaceutical compositions comprising an armed SVV, whereby the armed SVV encodes an agent for treating cancer. In certain embodiments, the pharmaceutical compositions may be supplemented with one or more agent disclosed above.

Provided herein is a pharmaceutical composition for treating a cancer in a subject in need thereof. The pharmaceutical composition comprises an armed SVV, and a pharmaceutically acceptable carrier; whereby the armed SVV encodes an agent for treating cancer.

Also provided herein is a pharmaceutical composition for treating a cancer in a subject in need thereof The pharmaceutical composition comprises an armed SVV, and a pharmaceutically acceptable carrier; whereby the armed SVV encodes an agent for treating cancer.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment provided herein, the pharmaceutical composition useful for practicing the method of the invention may be administered to deliver a dose of between 1 ng/kg/day and 100 mg/kg/day. In another embodiment, the pharmaceutical composition useful for practicing the invention may be administered to deliver a dose of between 1 ng/kg/day and 500 mg/kg/day. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In some embodiments, the SVV can be formulated in a natural capsid, a modified capsid, as a naked RNA, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In one aspect a pharmaceutical composition is disclosed for treating a cancer in a subject. The pharmaceutical composition comprises an armed SVV and a pharmaceutical acceptable carrier. Pharmaceutically acceptable carriers, which are useful, include, but are not limited to, glycerol, water, saline, ethanol, and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The compositions may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea, and combinations thereof. A particularly preferred preservative is a combination of about 0.5% to 2.0% benzyl alcohol and 0.05% to 0.5% sorbic acid.

The compositions may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The pharmaceutical composition disclosed herein may be used in combination with an additional therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

### Administration/Dosing

The armed SVV may be administered using suitable administration routes known to the art. In certain embodiments, the armed SVV is used in combination with an additional compound useful for treating cancer. In other embodiments, the armed SVV is used in conjunction with an SVV (which has not been armed).

In certain embodiments of the invention, the armed SVV and the additional compound useful for treating cancer are administered at the same time. In other embodiments, the additional compound is administered before the armed SVV is administered. In another embodiment, the additional inhibitor is administered after armed SVV administration.

The regimen of administration may affect what constitutes an effective amount. For example, the therapeutic formulations may be administered to the patient subject either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In general, SVV or armed SVV is administered in an amount of between 107 and 1×10¹¹ vp/kg. The exact dosage to be administered depends on a variety of factors including the age, weight, and sex of the patient, and the size and severity of the tumor being treated.

SVV or armed SVV is typically administered at a therapeutically effective dose. A therapeutically effective dose refers to that amount of the virus that results in amelioration of symptoms or a prolongation of survival in a patient. Toxicity and therapeutic efficacy of viruses can be determined by standard procedures in cell cultures or experimental animals, e.g., for determining the LD₅₀ (the dose lethal to 50% of the population of animals or cells; for viruses, the dose is in units of vp/kg) and the ED₅₀ (the dose, vp/kg, therapeutically effective in 50% of the population of animals or cells), or the TC₁₀ (the therapeutic concentration or dose allowing inhibition of 50% of tumor cells and can be related to PFU) or the EC₅₀ (the effective concentration, vp/cell, in 50% of the population of animals or cells). The dose ratio between toxic and therapeutic effects is the therapeutic index and it can be expressed as the ratio between LD₅₀ and ED₅₀ or EC₅₀. The dosage of viruses lies preferably within a range of circulating concentrations that include the ED₅₀ or EC₅₀ with little or no toxicity. The dosage may vary within this range depending upon the dosage form employed- and the route of administration utilized.

The SVV or armed SVV may be present in the composition in multidose and single dosage amounts, including, but not limited to between or between about 1×105 and 1×10¹² pfu, 1×10⁶ to 1×10¹⁰ pfu, or 1×10⁷ to 1×10¹⁰ pfu, each inclusive, such as at least, or about at least 1×10⁵, 1×10⁶, 1×10⁷, 1×10⁸, 1×10⁹, 2×10⁹, 3×10⁹, 4×10⁹, 5×10⁹, 6×10⁹7×10⁹, 8×10⁹, 9×10⁹, 1×¹⁰, 1×10¹¹, or 1×10¹² pfu.

The volume of the composition can be any volume, and can be for single or multiple dosage administration, including, but not limited to, from or from about 0.01 mL to 100 mL, 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, or 0.5 mL to 5 mL, each inclusive.

The infectivity of the SVV and/or armed SVV can be manifested, such as by increased titer or half-life of the oncolytic virus when exposed to a bodily fluid, such as blood or serum. Infectivity can be increased by any amount, including, but not limited to, at least 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold. 6-fold, 7-fold, 8-fold, 9-fold, or 10-fold.

Administration of the compositions of the present invention to a patient subject, preferably a mammal, more preferably a human, may be carried out using known procedures, at dosages and for periods of time effective to treat cancer in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic compound is from about 0.01 to about 50 mg/kg of body weight/per day.

The armed SVV can be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for treating cancer in a patient.

### Routes of Administration

One skilled in the art will recognize that although more than one route can be used for administration, a particular route can provide a more immediate and more effective reaction than another route. In one embodiment, the armed SVV is administered intratumorally.

Routes of administration of the disclosed compositions (containing an armed SVV) include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein. In one embodiment, the armed SVV treatment comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal, and any combination thereof.

### Kits

The invention also includes kits containing an armed SVV, whereby the kits are used to treat cancer.

In further embodiments a kit is provided for treating or ameliorating a cancer, as described elsewhere herein wherein the kit comprises: a) an armed SVV or composition comprising an armed SVV; and optionally b) an additional agent or therapy as described herein. The kit can further include instructions or a label for using the kit to treat or ameliorate the cancer. The kit can also include an assay to confirm that the cancer is indeed refractory to the checkpoint inhibitor. In yet other embodiments, the invention extends to kits assays for a given cancer (such as, but not limited to, small-cell lung cancer or triple negative breast cancer), as described herein. Such kits may, for example, contain the reagents from PCR or other nucleic acid hybridization technology (microarrays) or reagents for immunologically based detection techniques (e.g., ELISpot, ELISA).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention, and are not to be construed as limiting in any way the remainder of the disclosure.

### Example 1: Construction of Armed Seneca Valley Viruses

Hales et al., 2008 described the genome sequence of SVV-001. The RNA genome of SVV-001 consists of 7280 nt, excluding a 39 poly(A) tail, and has a 666 nt 5′ UTR and a shorter 3′ UTR (71 nt). The deduced amino acid sequence of the open reading frame (ORF) revealed a large, single ORF of 6543 bp with the potential to encode a polyprotein precursor of 2181 aa.

Poirer et al., 2012 describe the construction of pNTX-11, an SVV plasmid encoding GFP where the GFP cDNA is inserted between the SVV 2A and 2B coding sequences as indicated below. The Methods used by Poirer et al. are shown in Example 2.

The therapeutic cDNAs are inserted into the SVV-001 clone pNTX-11 deleted GFP where the GFP cDNA was excised and replaced by the therapeutic genes at nt. 3508. Thus, all the therapeutic gene insertions occur at nucleotide 3508 of pNTX-11 deleted GFP as indicated. To facilitate cloning, novel transgenes are synthesized to incorporate SVV sequences from the Nhe I (nt 3199) to Hind III (nt 4484) sites, allowing for facile cloning into the pNTX-11 deleted GFP backbone. The SVV genome starts at nt. 1 in pNTX-11 deleted GFP plasmid and the SVV ORF ends at 7281. pGEM 4Z plasmid sequences are found after the SVV 3′ UTR and (A)n sequence from nt. 7383-9885.

When generating the armed SVV using the procedure above, the nucleic acid sequence encoding the agent useful for treating cancer is used in place of the nucleic acid sequence of GFP. Specifically, the therapeutic cDNA is inserted into the SVV-001 clone pNTX-11 GFP where the GFP cDNA is excised and replaced by the therapeutic gene at nt. 3508. During translation of the SVV-therapeutic polyprotein, the ribosome will initially “skip” at the “TNPG↑P” motif of SVV 2A protein, and then once again at another “TNPG↑P” motif in the T2A protein. Two events of ribosomal skipping result in release of the therapeutic payload protein, flanked by one extra N-terminal proline and a C-terminal T2A cleavage product.

The parent plasmid for all the armed SVV viruses described in this Example is pNTX-11 deleted GFP, a 9,885 bp plasmid derived from pNTX-11, a 10,596 bp plasmid encoding GFP where the GFP cDNA was deleted from nt. 3508-4218. The SVV genome in pNTX-11 is contained in a pGEM-4Z plasmid backbone (Promega). Other parent plasmids may also be used.

Plasmids carrying armed SVV and a therapeutic protein have been constructed (see SEQ ID NOs: 15-18). These plasmid are as follows: pNTX-11 CXCL9 (SEQ ID NO: 15); pNTX-11+ TGFbDNRII (SEQ ID NO: 16); pNTX-11 nfsa mut 22 (SEQ ID NO: 17); and pNTX-11 Neoleukin 2-15 (SEQ ID NO: 18). In addition, a plasmid carrying epitopes for both chicken ovalbumin and Sars-Cov-2 (Covid virus) has been designed (pNTX-11 ova+covid epitopes (SEQ ID NO: 19)).

Table 1 shows the locations of the armed SVV and protein in the plasmids. A detailed description of each of these plasmids follows.

TABLE 1
Locations of SVV construct on plasmids

| SEQ | | Location of | Location |
|---|---|---|---|
| ID NO: | Description | SVV construct | of protein |
| 13 | pNTX-11 VHH aPDL-1 | 1-7762 | 3508-3885 |
| 14 | pNTX-11 IL2 quad mutant | 1-7783 | 3505-3906 |
| 15 | pNTX-11 CXCL9 | 1-7759 | 3508-3882 |
| 16 | pNTX-11 + TGFbDNRII | 1-7984 | 3508-4107 |
| 17 | pNTX-11 nfsa mut 22 | 1-8140 | 3508-4263 |
| 18 | pNTX-11 Neoleukin 2-15 | 1-7738 | 3508-3861 |
| 19 | pNTX-11 ova + covid | 1-7891 | 3508-4014 |
| | epitopes | | (ova + covid |
| | | | epitopes) |

### Construction of Armed SVV Encoding Anti PDL-1

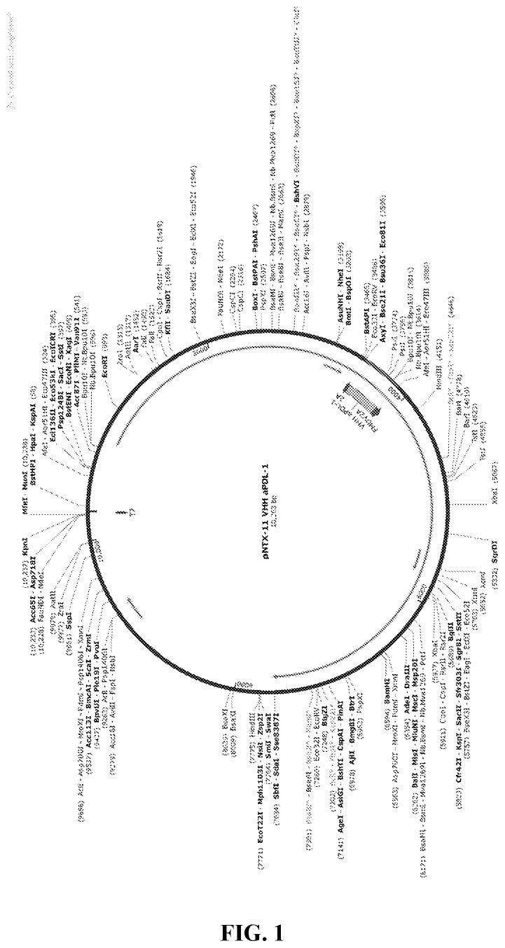

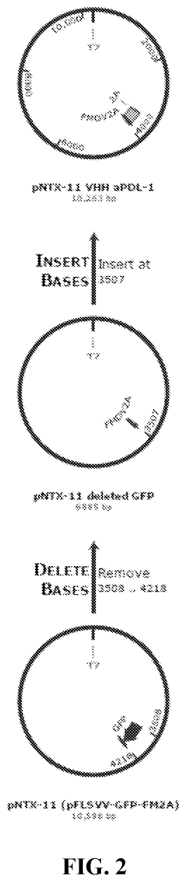

To construct an armed SVV encoding anti-PDL-1, the nucleotide sequence of a VHH nanobody encoding anti-PDL-1 (SEQ ID NO: 1) was inserted into pNTX-11 deleted GFP using the procedure described herein. The plasmid map of the resulting plasmid pNTX-11 VHH aPDL-1 is shown in FIG. 1. A schematic of the generating this plasmid (pNTX-11 VHH aPDL-1) is shown in FIG. 2. The resulting modified SVV expresses the anti-PDL1 protein (SEQ ID NO: 2).

nucleotide sequence of VHH anti-PD-L1
sequence (378 pb)
SEQ ID NO: 1
CAGGTGCAGCTGCAGGAGTCTGGAGGAGGCTCGGTGCAGACCGGA
GGGTCTCTGAGACTCTCCTGTACAGCCTCTACATCAATATACAGT
AACAACTACATGGCCTGGTTCAGCCAGTCTCCAGGAAAGGGGCGC
GAGGGGGTCGCAGCTGTTTATATGGATGATGGTCGCCCATACTAT
GCCGATTCCGTGAAGGGCCGATTCACCATCTCCCTAGACAGCGCC
AAGAACACGATGTATTTGCAAATGAACAGCCTGAAACCTGAGGAC
ACTGCCATGTACTACTGTGCGGCAGCTCCAGGCCCCTTAAGTCGT
AACTACTGGTACACGTCCGCCAACTATGACTACTGGGGCCAGGGG
ACCCAGGTCACCGTCTCCTCA
amino acid sequence of VHH anti-PD-L1
sequence.
SEQ ID NO: 2
QVQLQESGGGSVQTGGSLRLSCTASTSIYSNNYMAWFSQSPGKGR
EGVAAVYMDDGRPYYADSVKGRFTISLDSAKNTMYLQMNSLKPED
TAMYYCAAAPGPLSRNYWYTSANYDYWGQGTQVTVSS

With reference to the resulting plasmid (SEQ ID NO: 13) carrying the armed SVV construct, the SVV-anti-PD-L1 virus is from 1-7762. The anti-PD-L1 cDNA is from nt. 3508-3885. The sequence used to generate the SVV armed virus was derived from SEQ ID NO: 1 of WO 2017/157334 A.

### Construction of Armed SVV Encoding IL-2 Quadruple Mutant

The quadruple mutant 11-2 does not bind to CD25 (alpha receptor), but only binds CD122 and CD132 (beta and gamma chains) of IL-2 receptor. Removing the CD25 binding domain reduces T-suppressor generation and the potential for IL-2 toxicity mediated by vascular leak syndrome.

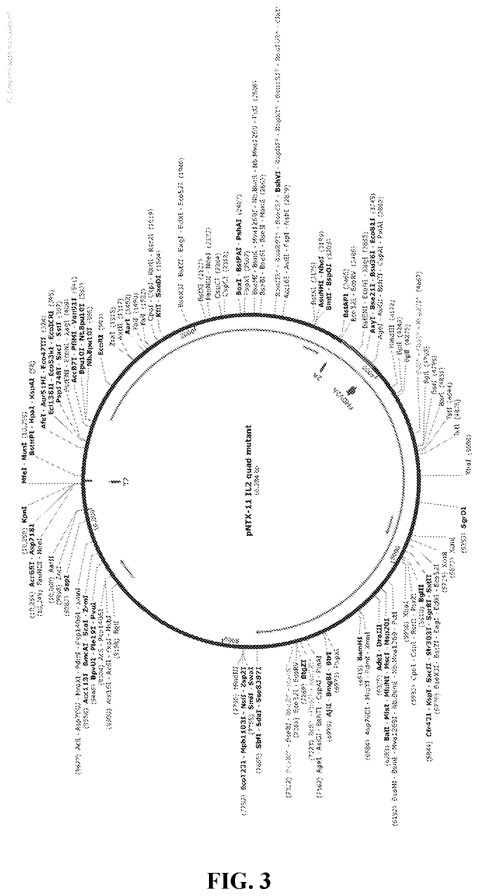

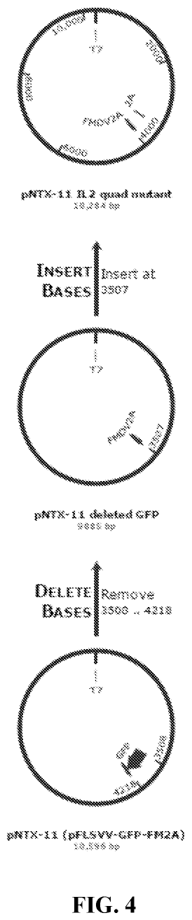

To construct an armed SVV encoding the IL-2 quadruple mutant, the nucleotide sequence of the IL-2 quad mutant (T3A/F42A/Y45A/L72G) (SEQ ID NO: 3) was inserted into pNTX-11 deleted GFP using the procedure described herein. The plasmid map of the resulting plasmid pNTX-11 IL2 quad mutant is shown in FIG. 3. A schematic of the generating this plasmid (pNTX-11 IL2 quad mutant) is shown in FIG. 4. The resulting modified SVV expresses the IL-2 quadruple mutant protein (SEQ ID NO: 4).

DNA sequence of IL-2 quadruple mutant
SEQ ID NO: 3
gctcctgcct cctccagcac caagaaaacc cagctccagc tggaacatct cctgctggat 60
ctgcagatga tcctgaacgg catcaacaac tacaagaacc ccaagctgac ccggatgctg 120
accgccaagt tcgccatgcc caagaaggcc accgagctga aacatctgca gtgcctggaa 180
gaggaactga agcctctgga agaggtgctg aacggcgccc agtccaagaa cttccacctg 240
aggcctcggg acctgatctc caacatcaac gtgatcgtgc tggaactgaa gggctccgag 300
acaaccttca tgtgcgagta cgccgacgag acagctacca tcgtggaatt tctgaaccgg 360
tggatcacct tcgcccagtc catcatctcc accctgacc 399
IL-2 quadruple mutant T3A/F42A/Y45A/L72G (C125A) amino-acid
sequence (133 amino acids)
SEQ ID NO: 4
Ala Pro Ala Ser Ser Ser Thr Lys Lys Thr Gln Leu Gln Leu Glu His Leu
Leu Leu Asp Leu Gln Met Ile Leu Asn Gly Ile Asn Asn Tyr Lys Asn Pro
Lys Leu Thr Arg Met Leu Thr Ala Lys Phe Ala Met Pro Lys Lys Ala Thr
Glu Leu Lys His Leu Gln Cys Leu Glu Glu Glu Leu Lys Pro Leu Glu Glu
Val Leu Asn Gly Ala Gln Ser Lys Asn Phe His Leu Arg Pro Arg Asp Leu
Ile Ser Asn Ile Asn Val Ile Val Leu Glu Leu Lys Gly Ser Glu Thr Thr
Phe Met Cys Glu Tyr Ala Asp Glu Thr Ala Thr Ile Val Glu Phe Leu Asn
Arg Trp Ile Thr Phe Ala Gln Ser Ile Ile Ser Thr Leu Thr

With reference to the resulting plasmid (SEQ ID NO: 14) carrying the armed SVV construct, the SVV IL-2 quad mutein virus sequence is from 1-7783. The IL-2 quad mutein cDNA sequence is from 3508-3906 in the SVV virus. The IL-2 quad mutein used to generate the armed SVV was derived from Ast et al., 2010, EP3075745B1, and U.S. Pat. No. 9,266,938B2.

### Construction of Armed SVV Encoding CXCL9 (417 bp)

CXCL9 is a chemokine thought to be involved in T cell trafficking. The encoded protein binds to C—X-C motif chemokine 3 and is a chemoattractant for lymphocytes but not for neutrophils. CXCL9 is also known as MIG-1 (Monokine Induced By Interferon-Gamma). CXCL9 has 125 amino acids and has a Molecular mass of 14,019 Da.

To construct an armed SVV encoding CXCL9, the nucleotide sequence of CXCL9 (SEQ ID NO: 5) was inserted into pNTX-11 deleted GFP using the procedure described herein. The GFP gene is resected from pNTX-11 and the CXCL9 cDNA is inserted into the SVV-001 clone at nt. 3508. During translation of the SVV-CXCL9 polyprotein, the ribosome initially skipped at the “TNPG↑P” motif of SVV 2A protein, and then did once again at another “TNPG↑P” motif in the T2A protein. Two events of ribosomal skipping result in release of the CXCL9 protein, flanked by one extra N-terminal proline and a C-terminal T2A cleavage product

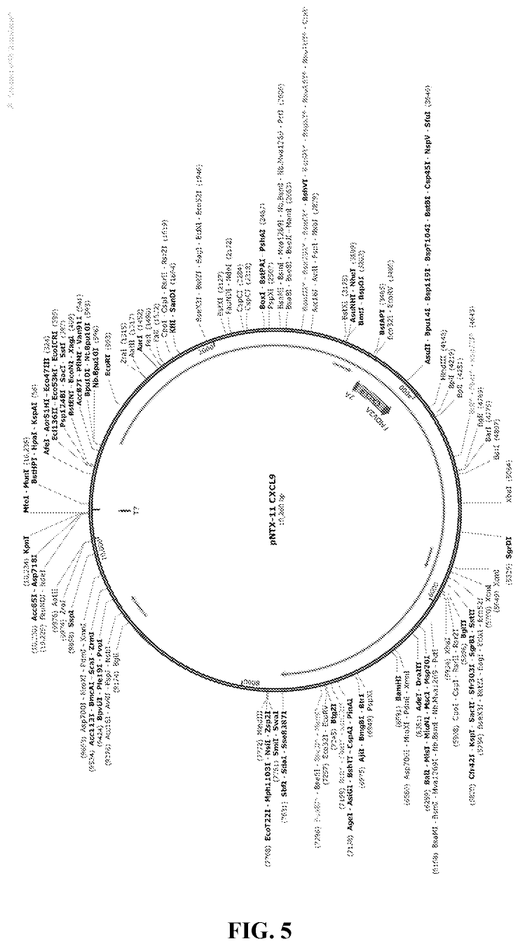

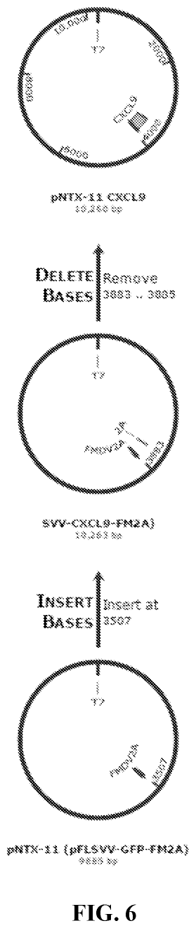

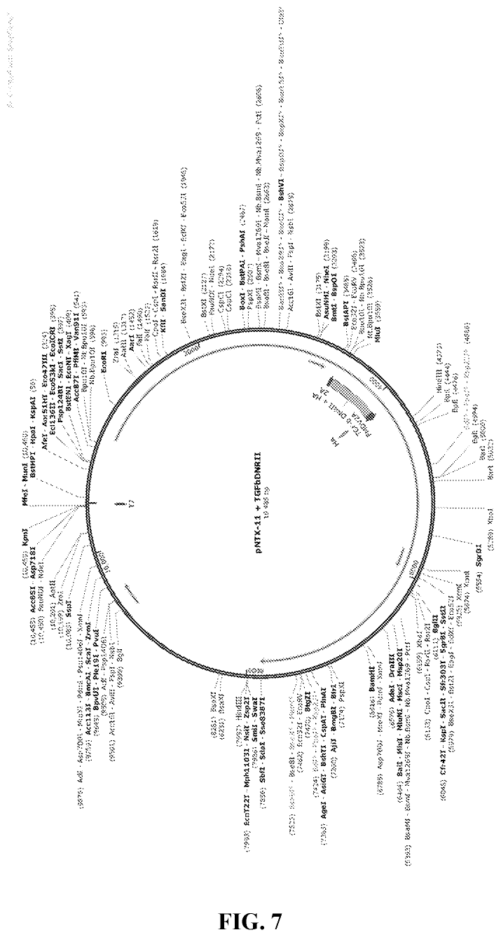

The plasmid map of the resulting plasmid pNTX-11 CXCL9 is shown in FIG. 5. A schematic of the generating this plasmid (pNTX-11 IL2 quad mutant) is shown in FIG. 6. The resulting modified SVV expresses the CXCL9 protein (SEQ ID NO: 6).

```
CXCL9 Nucleotide Sequence (Length: 378 bp)
SEQ ID NO: 5
    1 ATGAAGAAAA GTGGTGTTCT TTTCCTCTTG
        GGCATCATCT TGCTGGTTCT GATTGGAGTG
  61 CAAGGAACCC CAGTAGTGAG AAAGGGTCGC
        TGTTCCTGCA TCAGCACCAA CCAAGGGACT
121 ATCCACCTAC AATCCTTGAA AGACCTTAAA
        CAATTTGCCC CAAGCCCTTC CTGCGAGAAA
181 ATTGAAATCA TTGCTACACT GAAGAATGGA
        GTTCAAACAT GTCTAAACCC AGATTCAGCA
241 GATGTGAAGG AACTGATTAA AAAGTGGGAG
        AAACAGGTCA GCCAAAAGAA AAAGCAAAAG
301 AATGGGAAAA AACATCAAAA AAAGAAAGTT
        CTGAAAGTTC GAAAATCTCA ACGTTCTCGT
361 CAAAAGAAGA CTACATAA
CXCL9 protein sequence
SEQ ID NO: 6
MKKSGVLFLLGIILLVLIGVQGTPVVRKGRCSCISTNQG
THLQSLKDLKQFAPSPSCEKIEIIATLKNGVQTCLNPDS
ADVKELIKKWEKQVSQKKKOKNGKKHQKKKVLKVRKSQR
SRQKKTT
```

With reference to the resulting plasmid (SEQ ID NO: 15) carrying the armed SVV construct, the SVV CXCL9 virus sequence is from 1-7759. The CXCL9 cDNA sequence is from 3508-3882 in the SVV virus.

### Construction of Armed SVV Encoding TGF-Beta Decoy

For the construction of this armed SVV, a nucleic acid encoding the extracellular domains of human TGβRI ECD1-128 (384 bp) and TβRII ECD1-184 (486 bp) was used. TGF-β decoy receptors bind to TGF-β (e.g. TGF-β1, TGF-β2, and/or TGF-β3) and are derived from TGF-beta receptors lacking the amino acid sequence encoding a transmembrane domain. Expression of TGF-b decoys will reduce the immunosuppressive milieu in the tumor microenvironment and augment T cell responses.

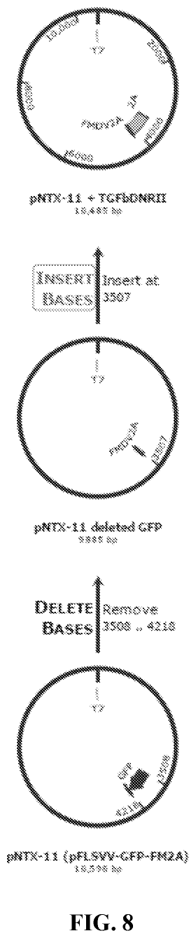

To construct an armed SVV encoding the TGF-beta decoy, the nucleotide sequence of a TGF-beta decoy (SEQ ID NO: 7) was inserted into pNTX-11 deleted GFP using the procedure described herein. The plasmid map of the resulting plasmid pNTX-11+TGFbDNRII is shown in FIG. 7. A schematic of the generating this plasmid (pNTX-11+TGFbDNRII) is shown in FIG. 8. The resulting modified SVV expresses the TGF-beta decoy protein (SEQ ID NO: 8). This construct has a myc tag appended at the COOH-tail of the protein. The sequence was obtained from: Addgene-plasmid-130888. The myc tag may be removed.

SEQ ID NO: 7:
TGF-beta decoy nucleotide sequence
ATGGGTCGGGGGCTGCTCAGGGGCCTGTGGCCGCTGCACATCGTC
CTGTGGACGCGTATCGCCAGCACGATCCCACCGCACGTTCAGAAG
TCGGTTAATAACGACATGATAGTCACTGACAACAACGGTGCAGTC
AAGTTTCCACAACTGTGTAAATTTTGTGATGTGAGATTTTCCACC
TGTGACAACCAGAAATCCTGCATGAGCAACTGCAGCATCACCTCC
ATCTGTGAGAAGCCACAGGAAGTCTGTGTGGCTGTATGGAGAAAG
AATGACGAGAACATAACACTAGAGACAGTTTGCCATGACCCCAAG
CTCCCCTACCATGACTTTATTCTGGAAGATGCTGCTTCTCCAAAG
TGCATTATGAAGGAAAAAAAAAAGCCTGGTGAGACTTTCTTCATG
TGTTCCTGTAGCTCTGATGAGTGCAATGACAACATCATCTTCTCA
GAAGAATATAACACCAGCAATCCTGACTTGTTGCTAGTCATATTT
CAAGTGACAGGCATCAGCCTCCTGCCACCACTGGGAGTTGCCATA
TCTGTCATCATCATCTTCTACTGCTACCGCGTTTACCCATACGAT
GTTCCAGATTACGCT
SEQ ID NO: 8:
TGF-beta decoy protein sequence
MGRGLLRGLWPLHIVLWTRIASTIPPHVQKSVNNDMIVTDNNGAV
KFPQLCKFCDVRESTCDNQKSCMSNCSITSICEKPQEVCVAVWRK
NDENITLETVCHDPKLPYHDFILEDAASPKCIMKEKKKPGETFFM
CSCSSDECNDNIIESEEYNTSNPDLLLVIFQVTGISLLPPLGVAI
SVIIIFYCYRVYPYDVPDYA

With reference to the resulting plasmid (SEQ ID NO: 16) carrying the armed SVV construct, the SVV TGF-beta decoy virus sequence is from 1-7984. The TGF-beta decoy cDNA sequence is from 3508-4107 in the SVV virus.

### Construction of Armed SVV Expressing NfsA

Gene-directed enzyme prodrug therapy (GDEPT) is a developing strategy for cancer treatment, involving delivery to tumor cells of an exogenous gene, encoding an enzyme that can convert a non-toxic prodrug into cytotoxic products. In principle, local generation of highly reactive cytotoxins within the cancer cells allows optimal therapeutic effect, whereas systemic toxicity remains lower than with conventional chemotherapy. The nitroreductase NfsB from Escherichia coli can activate the prodrug, CB1954, to a potent bifunctional alkylating agent. NfsA preferentially reduces the 2-NO₂ group of CB1954, resulting in an improved bystander cell killing compared to Nfsb. Overall, the results suggest that NfsA could have advantages over NfsB for use in GDEPT with CB1954 or several other nitroaromatic prodrugs.

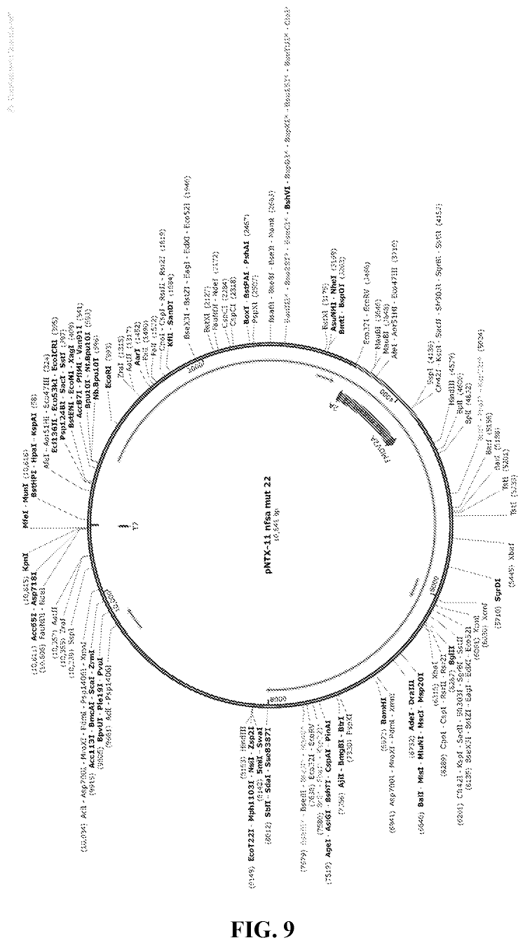

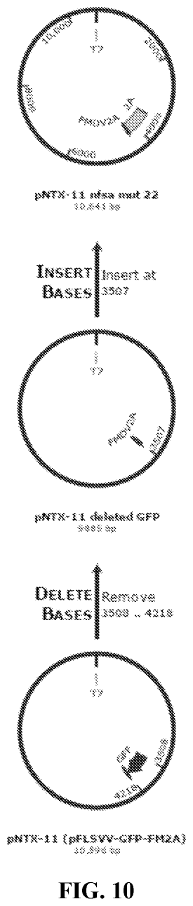

To construct an armed SVV encoding NfsA, the nucleotide sequence of a NfsA (SEQ ID NO: 9) was inserted into pNTX-11 deleted GFP using the procedure described herein. The plasmid map of the resulting plasmid pNTX-11 nfsa mut 22 is shown in FIG. 9. A schematic of the generating this plasmid (pNTX-11 nfsa mut 22) is shown in FIG. 10. The resulting modified SVV expresses the Nfsa protein (SEQ ID NO: 10).

Nucleotide sequence of NfsA
SEQ ID NO: 9
AGCGCGATGCCGCTGGAAAGCGAACAGGAAAACTGCGAAATGACC
CCGACCATTGAACTGATTTGCGGCCATCGCAGCATTCGCCATTTT
ACCGATGAACCGATTAGCGAAGCGCAGCGCGAAGCGATTATTAAC
AGCGCGCGCGCGACCAGCAGCAGCTATTTTCTGCAGTGCAGCAGC
ATTATTCGCATTACCGATAAAGCGCTGCGCGAAGAACTGGTGACC
CTGACCGGCGGCCAGAAACATGTGGCGCAGGCGGCGGAATTTTGG
GTGTTTTGCGCGGATTTTAACCGCCATCTGCAGATTTGCCCGGAT
GCGCAGCTGGGCCTGGCGGAACAGCTGCTGATGGGCTGGGATACC
GCGATGATGGCGCAGAACGCGCTGATTGCGGCGGAAAGCCTGGGC
CTGGGCGGCGTGTATATTGGCGGCCTGCGCAACAACATTGAAGCG
GTGACCAAACTGCTGAAACTGCCGCAGCATGTGCTGCCGCTGTTT
GGCCTGTGCCTGGGCTGGCCGGCGGATAACCCGGATCTGAAACCG
CGCCTGCCGGCGAGCATTCTGGTGCATGAAAACAGCTATCAGCCG
CTGGATAAAGGCGCGCTGGCGCAGTATGATGAACAGCTGGCGGAA
TATTATCTGACCCGCGGCAGCAACAACCGCCGCGATACCTGGAGC
GATCATATTCGCCGCACCATTATTAAAGAAAGCCGCCCGAGCATT
CTGGATTATCTGCATAAACAGGGCTGGGCGACCCGC
amino-acid sequence of NfsA
SEQ ID NO: 10
MTPTIELICGHRSIRHFTDEPISEAQREAIINSARATSSSYFLQC
SSIIRITDKALREELVTLTGGQKHVAQAAEFWVFCADFNRHLQIC
PDAQLGLAEQLLMGWDTAMMAQNALIAAESLGLGGVYIGGLRNNI
EAVTKLLKLPQHVLPLFGLCLGWPADNPDLKPRLPASILVHENSY
QPLDKGALAQYDEQLAEYYLTRGSNNRRDTWSDHIRRTIIKESRP
SILDYLHKQGWATR

With reference to the resulting plasmid (SEQ ID NO: 17), the SVV Nfsa virus sequence is from 1-8140. The Nfsa cDNA sequence is from 3508-4263 in the SVV virus.

The NsFA sequence used to construct this armed SVV was derived from SEQ ID NO: 32 from WO2012008860. See also Vass, S., Jarrom, D., Wilson, W. et al. E. coli NfsA: an alternative nitroreductase for prodrug activation gene therapy in combination with CB1954. Br J Cancer 100, 1903-1911 (2009). https://doi.org/10.1038/sj.bjc.6605094

### Construction of Armed SVV Expressing Neoleukin 2-15

Neoleukin 2-15 is an improved IL-2 mutant which lacks binding site for IL-2Rα (also called CD25) or IL-15Rα (also known as CD215). The molecule is hyper-stable, binds human and mouse IL-2Rβγc with higher affinity than the natural cytokines and is more potent.

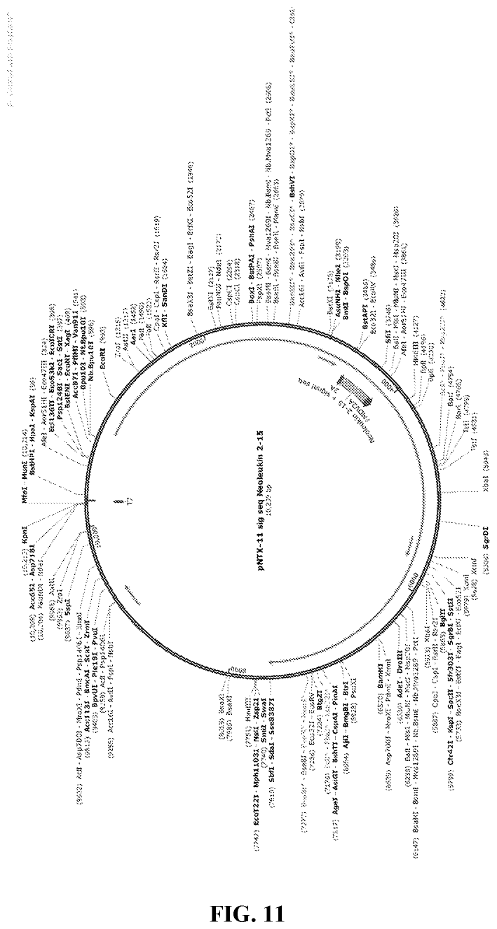

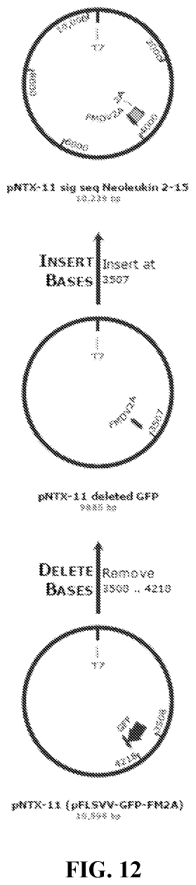

To construct an armed SVV encoding Neoleukin 2-15, the nucleotide sequence of a Neoleukin 2-15 (SEQ ID NO: 11) was inserted into pNTX-11 deleted GFP using the procedure described herein. The plasmid map of the resulting plasmid pNTX-11 sig seq Neoleukin is shown in FIG. 11. A schematic of the generating this plasmid (pNTX-11 sig seq Neoleukin) is shown in FIG. 12. The resulting modified SVV expresses the Neoleukin 2-15 (SEQ ID NO: 12).

DNA sequence of Neoleukin 2-15
SEQ ID NO: 11
ATGAAGTGGGTGACCTTCATCAGCCTGCTGTTCCTGTTCAGCAGC
GCCTACAGCCCCAAGAAGAAGATCCAGCTGCACGCCGAGCACGCC
CTGTACGACGCCCTGATGATCCTGAACATCGTGAAGACCAACAGC
CCCCCCGCCGAGGAGAAGCTGGAGGACTACGCCTTCAACTTCGAG
CTGATCCTGGAGGAGATCGCCAGACTGTTCGAGAGCGGCGACCAG
AAGGACGAGGCCGAGAAGGCCAAGAGAATGAAGGAGTGGATGAAG
AGAATCAAGACCACCGCCAGCGAGGACGAGCAGGAGGAGATGGCC
AACGCCATCATCACCATCCTGCAGAGCTGGATCTTCAGC
Amino acid sequence of Neoleukin 2-15
SEQ ID NO: 12
MKWVTFISLLFLESSAYSPKKKIQLHAEHALYDALMILNIVKTNS
PPAEEKLEDYAFNFELILEEIARLFESGDQKDEAEKAKRMKEWMK
RIKTTASEDEQEEMANAIITILQSWIES

With reference to the result plasmid (SEQ ID NO: 18), the SVV Neoleukin 2-15 virus sequence is from 1-7738. The Neoleukin 2-15 cDNA sequence is from 3508-3861 in the SVV virus.

The sequences used to generate this construct were derived from Silva D A, Yu S, Ulge U Y, et al. De novo design of potent and selective mimics of IL-2 and IL-15. Nature. 2019; 565(7738):186-191.

### Construction of SVV Immunogenic Construct

A novel armed virus encoding multiple immunogenic epitopes from chicken ovalbumin, including SIINFEKYL and an immunogenic COVID peptide: gly-pro-lys-lys-ser-thr-asn-leu was designed. The immunogenic Covid murine H2-D^{d}-restricted CD8+ CTL epitope GPKKSTNL (aa 526-533) from the SARS-CoV-2 spike (S) protein is described by Muraoka et al., 2020.

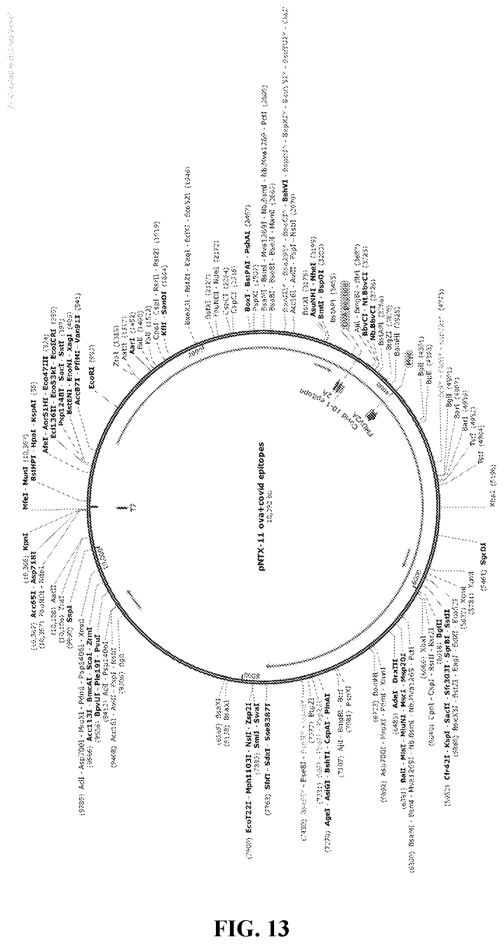

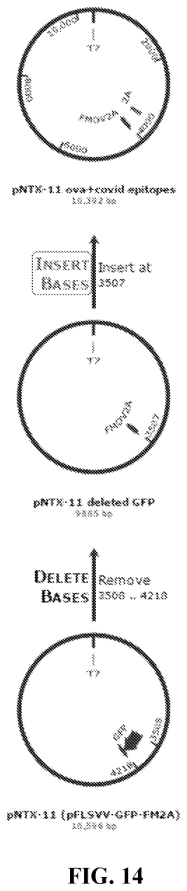

A map of the plasmid carrying this SVV construct (NTX-11 ova+covid epitope) is shown in FIG. 13. FIG. 14 shows a schematic of generating this plasmid. With reference to the resulting plasmid (SEQ ID NO: 19), the SVV ova+covid epitopes virus sequence is from 1-7891. The ova+covid epitopes cDNA sequence is from 3508-4014 in the SVV virus

### Example 2: Cloning of Full-Length SVV cDNA, Insertion, and Rescue of SVV-GFP

Synthesis and cloning of the full-length SVV-001 genome into a bacterial plasmid was described previously (Poirier et al., 2012). Generation of a GFP-expressing derivative of SVV-001 http://vir.sgmjournals.org 2611, the disclosure of which is incorporated herein).

Briefly, three cDNA fragments representing the full-length SVV001 genome were amplified by three PCR reactions employing six sets of SVV001-specific primers (see Table 2).

TABLE 2
Oligonucleotide primers used for the
construction of SVV-001 infectious
cDNA clone.

| | | SEQ |
|---|---|---|
| | Primer Sequence | ID |
| Primer ID | (5′ → 3′) | NO |
| 5'SVV-001- | TTTGAAATGGGGGGCTGGGC | 20 |
| SVV-0011029RT-RI | GAGGAGACCCGCTAATCCG | 21 |
| Nde-ApaT7SVV-001 | TATGGGTACCTGTAATACGA | 22 |
| | CTCACTATAGGGCTTTGAAA | |
| | TGGGGGGCTGGGCC | |
| SVV-0016056 | CCGTCAAAGAAGCAATTCTG | 23 |
| | GGCA | |
| SVV-0017309NsiB | GCATGCATTTTTTTTTTTTTT | 24 |
| | TTTTTTTTTTTTTTTTCCCTT | |
| | TTCTGTTCCGACTGAGTT | |
| SVV-001911L | GGTAACATGACCTTCAATTAC | 25 |
| | TACGCAAAC | |
| SVV-0016157R | GATCAGTACGTCGAAGGCCGT | 26 |
| | TG | |
| SVV-0013SwaRev | GCTTGCATGCATTTAAATTTT | 27 |
| | TTTTTTTTTTTTTTTTTTTTT | |
| | TTTTTTCCCTTTTCTGTTCC | |
| | GACTGAGTTCTCCC | |
| Kpn-Mun- | CAATTGTGTAATACGACTCAC | 28 |
| ApaT7SVV-001 | TATAGTTTGAAATGGGGGGCT | |
| | GGGCC | |

Turbo Pfu polymerase (Stratagene) was used in the PCRs. First, a fragment representing the 5′ end of the SVV-001 genome was amplified with primers 59SVV-001-A and SVV0011029RT-RI and the resulting fragment was cut with ApaI and EcoRI and gel purified. The gel-purified fragment was ligated to NdeApa T7SVV-001, an annealed oligonucleotide duplex containing an engineered NdeI site at the 5′ end, a T7 core promoter sequence in the middle and the first 17 nt of SVV-001 with an ApaI site at the 39 end, and cloned into the NdeI and EcoRI sites of pGEM-3Z (Promega) by three-way ligation to generate pNTX-03.

Next, a fragment representing the 3′ end of the viral genome was amplified by PCR with primers SVV-0016056 and SVV-0017309NsiB. The reverse primer, SVV0017309NsiB was used to introduce a poly(A) tail of 30 nt in length and an NsiI recognition sequence at the 3′ end to clone into the PstI site of the pGEM-3Z plasmid. The resulting PCR product was digested with BamH-I and gel purified.

A fragment covering an internal portion of the viral genome was amplified with primers SVV-0019111L and SVV0016157R. The resulting PCR product was digested with EcoRI and BamHI and gel purified. The two gel-purified fragments representing the middle and 3′ end of SVV genome were cloned into the EcoRI and SmaI sites of pGEM-4Z by three-way ligation to generate pNTX-02.

To generate the full-length SVV-001 cDNA, pNTX-02 was digested with EcoRI and NsiI and the resulting 7.3 kb fragment was gel purified and cloned into the EcoRI and PstI sites of pNTX-03. The resulting full-length plasmid was called pNTX-04. pNTX-04 was further modified at both the 5′ and 3′ ends to facilitate in vitro transcription and rescue of the virus following RNA transfection into PER.C6 cells.

First, a SwaI restriction enzyme site was inserted immediately downstream of the poly(A) tail to liberate the 3′ end of SVV-001 cDNA from the plasmid backbone prior to in vitro transcription and to provide a blunt end for termination. A PCR approach was used to insert the site utilizing the primer pair SVV-0016056 and SVV0013SwaRev and pNTX-04 as a template. The reverse primer SVV0013SwaRev contained 58 nt representing the 3′ end of the SVV-001 genome and recognition sequences for SwaI and SphI restriction enzyme sites. The resulting PCR fragment was digested with BamHI and SphI and used to replace the corresponding fragment from pNTX04 to generate pNTX-06. Next, the four extra nucleotides present between the T7 promoter transcription start site and the 59 end of the SVV-001 cDNA in pNTX-06 were removed using an annealed oligonucleotide duplex approach. The duplex oligonucleotides were engineered to contain a KpnI recognition site, T7 core promoter sequence and the first 17 nt of SVV-001 with an ApaI site at 3′ end. The annealed oligonucleotides were used to replace the corresponding portion of pNTX-06 using KpnI and ApaI sites to generate pNTX-07. Finally, a 2 bp deletion observed in the 3D polymerase-encoding region of pNTX-07 was restored by replacing the BamHI and SphI fragment with a corresponding fragment amplified from SVV-001 cDNA by PCR to generate pNTX-09.

Insertion of GFP-Coding Sequences into the Full-Length SVV001 Plasmid.

To insert a GFP-coding sequence fused to the F2A protein in between the SVV-001 2A- and 2B-coding regions, overlap extension PCR was used. Six primers were designed, each having overlap sequences to amplify three individual PCR fragments. The first PCR fragment (PCR a-b) was amplified using forward primer NI-03 binding upstream of 2A sequences and reverse primer NI-04 with 18 bp of 2A sequence, 3 bp of 2B sequence and 15 bp of the 5′ GFP sequence. A second PCR fragment (PCR c-d) with GFP-coding sequences was amplified with forward primer NI-05 having 9 bp of 2A sequence, 3 bp of 2B sequence and 29 bp of the GFP 5′ sequence and reverse primer (NI-06) with 21 bp of the GFP 3′ sequence and 48 bp of the F2A sequence. A third PCR fragment (PCR e-f) was amplified with forward primer NI-07 containing 46 bp of F2A sequence and 24 bp of 2B sequence and reverse primer NI-08 binding 615 bp downstream of the SVV-001 2A sequence. PCR fragments a-b and c-d were fused by amplification using primers NI-03 and NI-06 to generate the PCR a-d fragment. Finally, the PCR a-d and e-f fragments were fused by amplification using primers NI-03 and NI-08 to generate the PCR a-f fragment. The PCR a-f fragment was digested with NheI and HindIII and inserted into the corresponding sites in pNTX-09 to generate pNTX-11, an SVV full-length plasmid containing GFP-coding sequences fused to F2A.

### In Vitro Transcription and Infectivity of RNA.

The infectivity of in vitro-transcribed RNA was tested by first digesting pNTX-09 with SwaI to liberate the 3′ end of the SVV-001 sequence from the plasmid backbone. The linearized plasmid was subjected to in vitro transcription using T7 RNA polymerase (Promega). To assess transfection of in vitro-transcribed RNA in SVV-permissive cells, PER.C6 cells were plated in six-well tissue culture dishes. On the following day, Lipofectamine reagent (Invitrogen) was used to transfect in vitro transcribed RNA (1.5 mg) into the cells following the recommendations of the supplier. A CPE due to virus production was seen by 36 h post-transfection. The transfected cells were subjected to three freeze-thaw cycles, and the presence of virus in lysates was further confirmed by infecting PER.C6 cells.

### Results

A cDNA encoding the full-length wild-type SVV-001 genome was cloned into the pGEM-4Z expression vector as described above. To generate a recombinant reporter virus expressing GFP, a fusion protein of GFP and the F2A protein was cloned following the SVV-001 2A (S2A) protein in pNTX-09 to yield pNTX-11 as depicted in FIGS. 15A-C.

The F2A sequence was chosen over repetition of the S2A sequence to guard against unwanted recombination events between duplicated sequences. During translation of the SVV-GFP polypeptide, the ribosome skips at TNPGQP of the S2A sequence, continues in frame to produce a GFP-F2A fusion protein with one additional N-terminal proline from SVV-001 2B (S2B), skips a second time at the F2A SNPGQP sequence and continues in frame a second time to translate the remainder of the SVV-001 polyprotein. One clear advantage of this strategy is that all SVV proteins produced retain their native sequence. pNTX-11 was digested with SwaI and used as a template for in vitro transcription. RNA transcripts were transfected into ten 15 cm dishes of PER.C6 cells. A GFP expressing plaque was observed and purified. Plaque-purified and amplified SVV-GFP was used to infect SCLC H446 cells. A cytopathic effect (CPE) typical of wild-type SVV-001 infection was observed, as well as bright green fluorescence. Individual infected cells were bright enough to be detected easily over 4 logs by flow cytometry or as plaques of varying sizes by fluorescence scanning. Protein was extracted from H446 cells infected with SVV001 or SVV-GFP and Western blotted for GFP, the F2A epitope and glyceraldehyde 3-phosphate dehydrogenase. A strong signal for both GFP and F2A was detected at 30 kDa, corresponding to the GFP-F2A fusion protein.

The approach described above can be adapted to incorporation of therapeutic transgenes. Successful generation of SVV-GFP indicates that transgenes of up to 800 bp can be accommodated.

### Example 3: Development of Mammalian T7 Polymerase Cell Line (PerC.6-T7 #2)

The current system to generate armed SVV constructs requires the following steps: (1) construct armed SVV plasmid; (2) linearize armed SVV plasmid to define 3′ end; (3) in vitro transcription reaction using T7 polymerase to generate RNA transcript with authentic 5′ and 3′ termini; (4) transfect RNA into target cells; and (5) isolate armed SVV virus

A new system has been devised to generate armed SVV constructs quickly. The new system relies on the mammalian T7 polymerase cell line (PerC.6-T7 #2). The new system requires the following steps: (1) develop T7 polymerase plasmid optimized for expression in mammalian cells; (2) clone T7 polymerase optimized mammalian expression plasmid into target cell line and select best clone (PerC.6-T7 #2); (2) linearize armed SVV plasmid; (3) transfect plasmid into T7-pol cells; and (4) isolate the armed SVV virus.

Using both the current and new system, armed SVV constructs were designed and armed SVV viruses produced. The armed SVV viruses from both systems functioned equivalently.

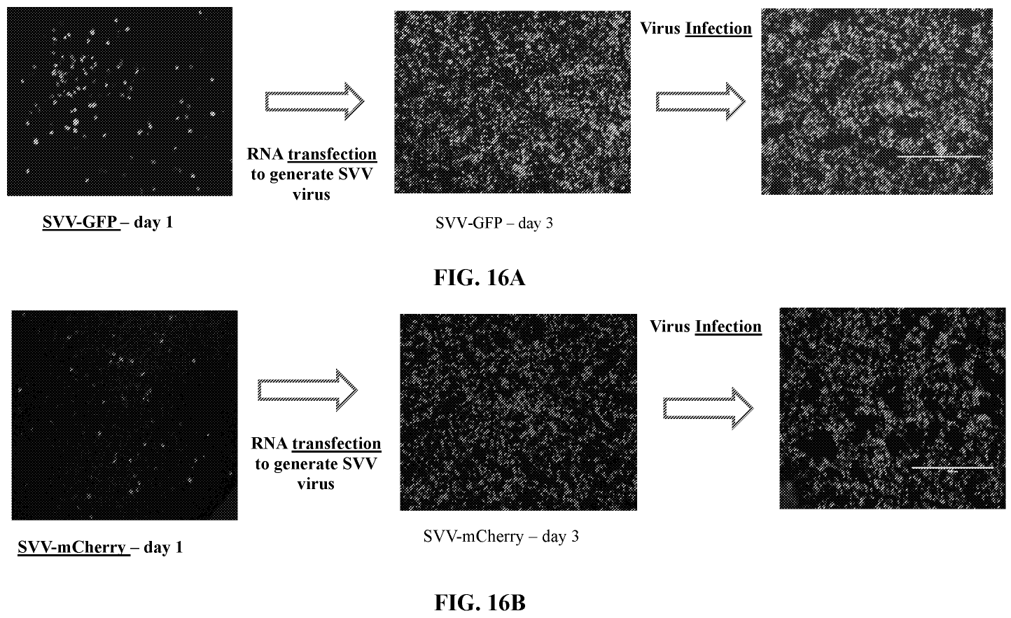

FIG. 16A and FIG. 16B show the rapid generation of armed SVV viruses in cell lines using the new system (PerC.6-T7 #2). FIG. 16A shows the results for SVV-GFP (SVV engineered to express GFP and FIG. 16B shows the results for SVV-mCherry (SVV engineered to express mCherry). This data demonstrates that it is possible to generate a variety of SVV constructs using the current system.

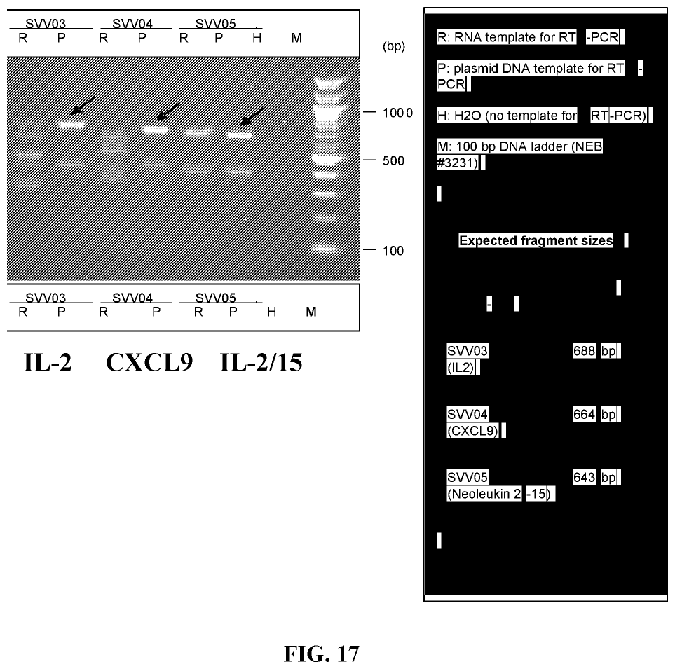

Using the current system, armed SVV constructs expressing IL-2, CXCL9, and IL-2/15 were generated. FIG. 17 shows the RT-PCR data for armed SVV generated to express IL-2, CXCL9, and IL-2/15. The RT-PCR data demonstrates that the armed SVV express therapeutic transgenes.

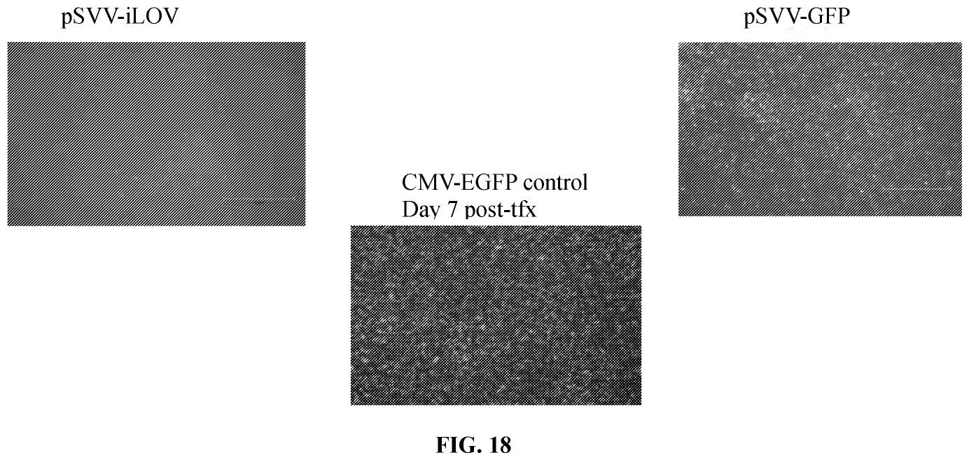

Furthermore, using the new system, an armed SVV construct carrying GFP was designed. FIG. 18 shows the transfection of linearized DNA in PerC-T7 pol cells. As is evident from FIG. 18 it is possible to generate armed SVV constructs using the PerC6-T7 pol cells.

### Example 4: Testing of SVV Armed with IL-2 and IL-2/-15

Interleukin-2 (IL-2), originally described as “T-cell growth factor” in 1976, is a small 15.5 kDa monomer secreted by a variety of cell types including CD4+ and CD8+ T cells, natural killer (NK) cells and activated dendritic cells. IL-2 has pleiotropic effects on the immune system. It plays a critical role in the generation, maintenance, and expansion of CD4+regulatory T cells, promotes the cytotoxic activity of NK and CD8+cells and governs homeostasis through the elimination of harmful autoreactive T cells via activation-induced cell death. IL-2 can signalize either via intermediate-affinity dimeric CD122/CD132 IL-2R or the high-affinityIL-2 Receptor composed of trimeric CD25/CD122/CD132.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (CD132). IL-15 is constitutively expressed by a large number of cell types and tissues. This cytokine induces the proliferation of natural killer cells, i.e. cells of the innate immune system whose principal role is to kill virally infected cells. IL-15 is a pleiotropic cytokine, it plays an important role in innate and adaptive immunity. IL-15 has been shown to enhance the anti-tumor immunity of CD8+ T cells.

Armed SVV constructs engineered to express IL-2 and IL-2/ -15 were generated and tested for the activity. The constructs were generated using the general procedures described in Example 1 above.

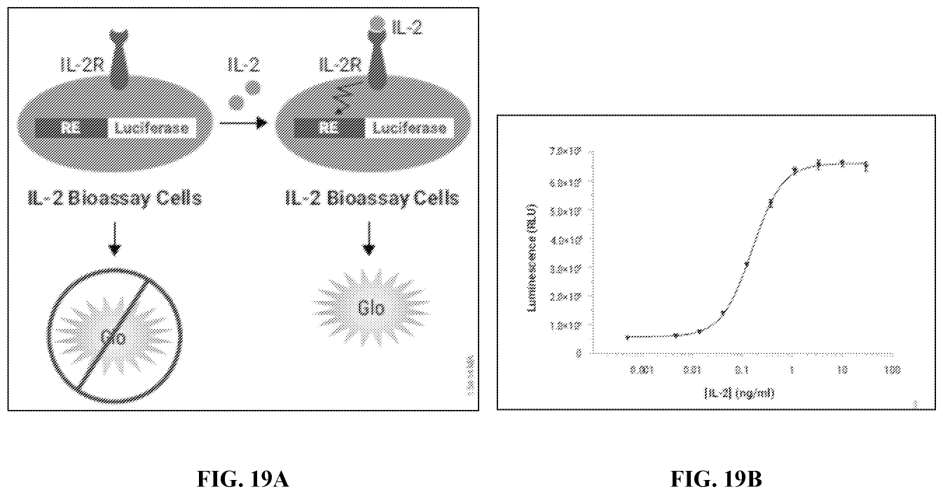

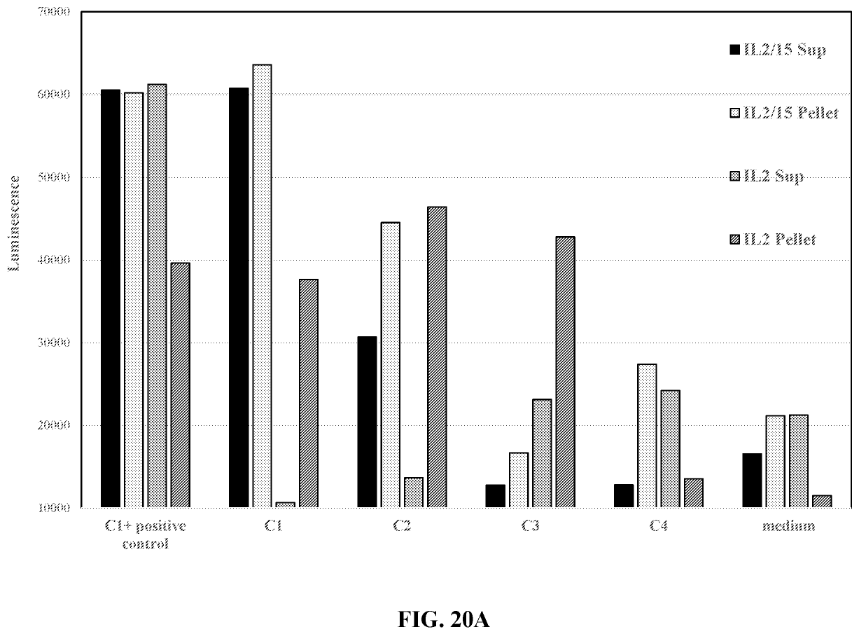

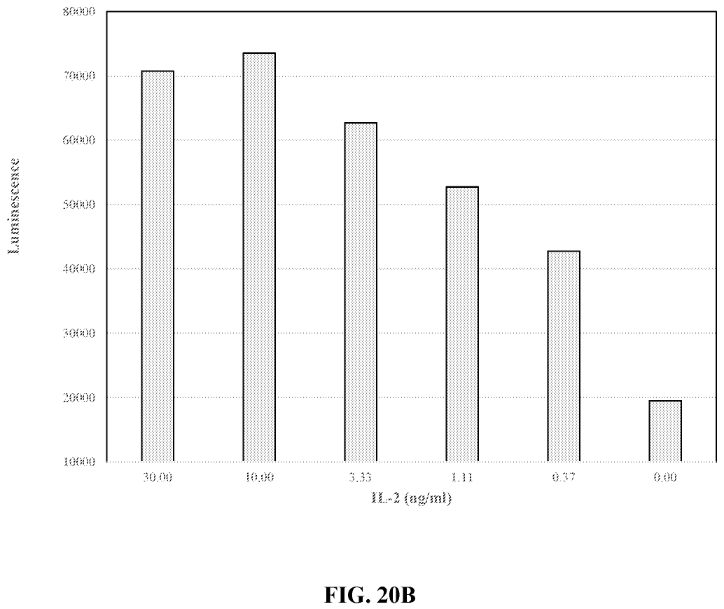

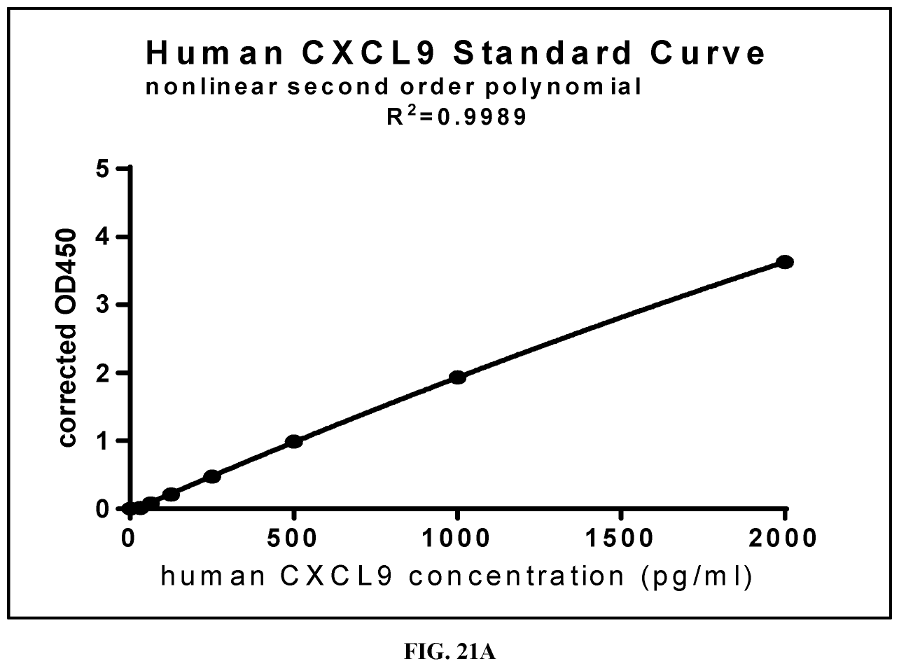

The IL-2 Bioassay Cells have been engineered to express luc2 in response to IL-2 signaling. When IL-2 binds to IL-2 Bioassay Cells, the receptor transduces intracellular signals that result in luminescence. The bioluminescent signal is detected and quantified. In the absence of IL-2, no signaling occurs downstream of IL-2R and no luminescent signal is generated. The IL-2 receptor (IL-2R) is composed of 3 subunits: IL-2Rα (CD25), IL-2RP (CD122) and IL-2Rγ (CD132). IL-15 signals via IL-2RP (CD122) and IL-2Rγ (CD132). Thus the IL-2 bioassay cells can detect cytokine binding and signaling via IL-2 or IL-15 receptors. These bioassays are shown in FIG. 19A and FIG. 19B.

Using these bioassays, the activity of the armed SVV constructs was assessed. The results of this testing are shown in FIG. 20A and FIG. 20B. FIG. 20A shows the activity for SVV-IL2 and SVV-IL2/15 (Neoleukin 2-15). FIG. 20B shows the activity for the IL-2 standard positive control. SVV-IL2/15 (Neoleukin 2-15) protein activity is in supernatant and pellet. SVV-IL-2 protein activity is in pellet only. This testing demonstrated that SVV can be transfected to express IL-2 and IL-2/15 (Neuleukin 2-15), which is expressed in the cells upon transfection with SVV.

### Example 5: Testing of SVV Armed with CXCL9

Chemokine (C—X-C motif) ligand 9 (CXCL9) is a small cytokine belonging to the CXC chemokine family that is also known as monokine induced by gamma interferon (MIG). The CXCL9 is one of the chemokines which plays role to induce chemotaxis, promote differentiation and multiplication of leukocytes, and cause tissue extravasation.

The CXCL9/CXCR3 receptor regulates immune cell migration, differentiation, and activation. Anti-tumor immune reactivity occurs through recruitment of immune cells, such as cytotoxic lymphocytes (CTLs), natural killer (NK) cells, NKT cells, and macrophages. CXCL9 predominantly mediates lymphocytic infiltration to tumor sites and suppresses tumor growth.

PerC.6-T7 #2 cells were transfected with plasmid pSVV-CXCL9 and supernatant (S) and pellet lysate (PS) were used to infect fresh PerC.6 cells. Supernatant samples were collected on days 1, 2, 3 (D1-3) and tested using CXCL9 ELISA.

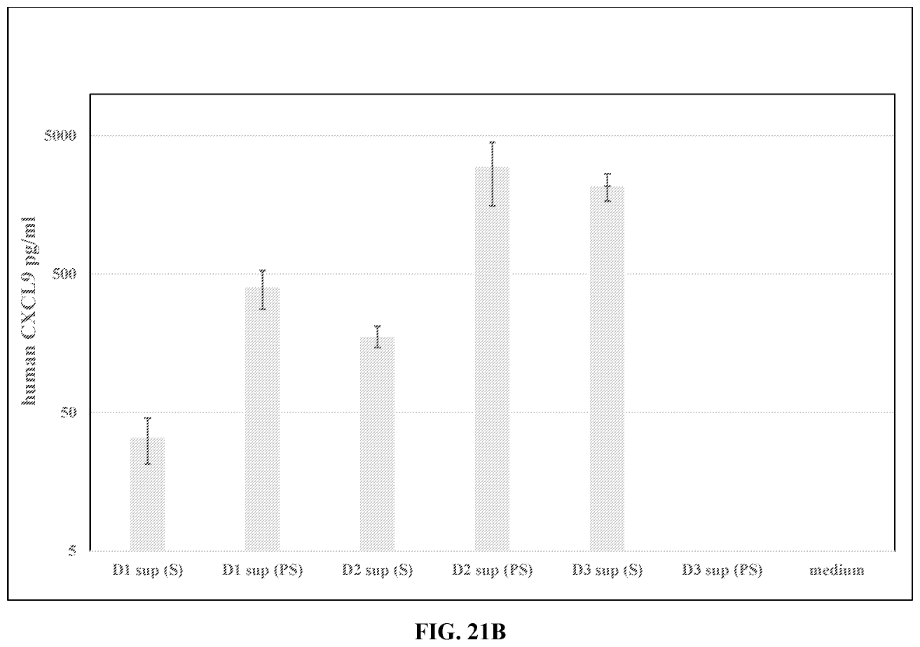

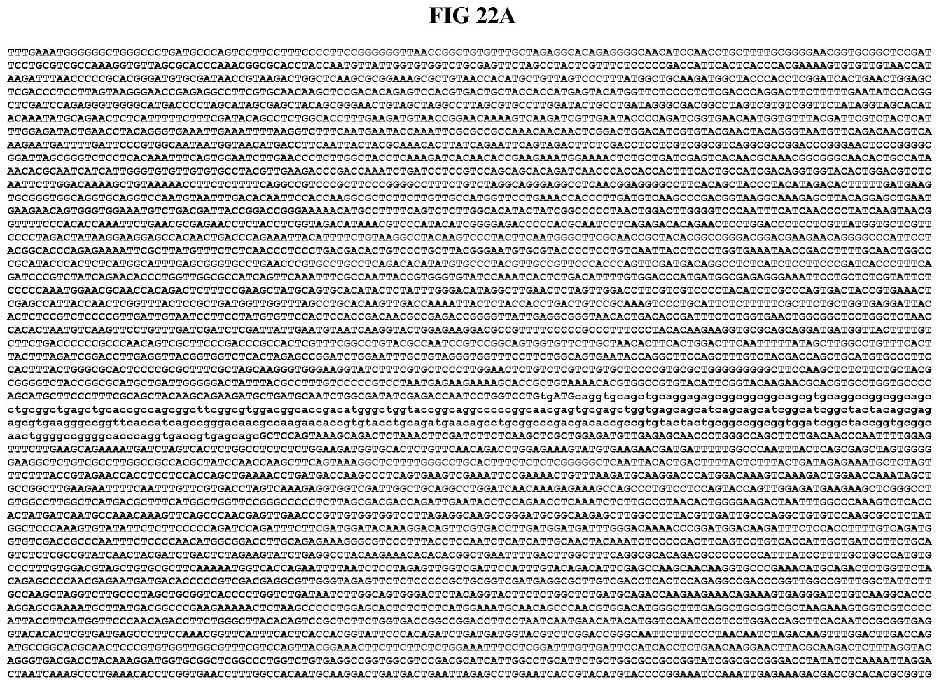

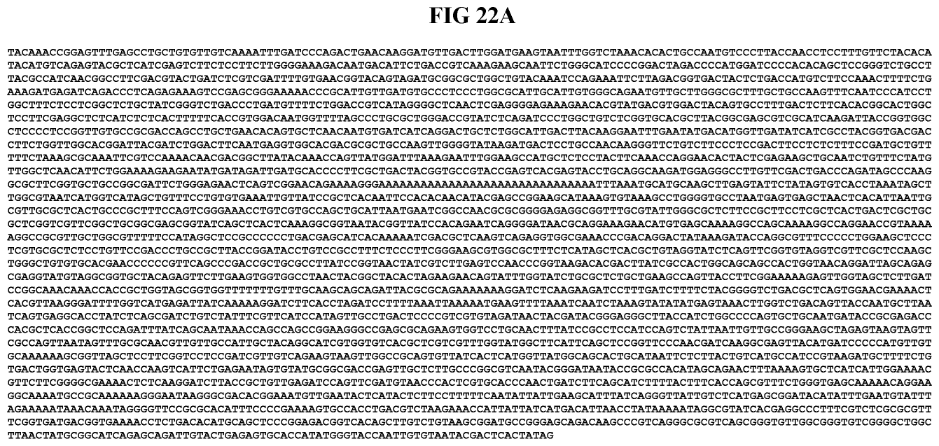

The results of this testing are shown in FIG. 21A and FIG. 21B. FIG. 21A and FIG. 21B show the data for SVV-CXCL9 Elisa. FIG. 21A shows the human CXCL standard curve. FIG. 21B shows the human CXCL9 level detection in amplified SVV-CXCL9 supernatants. Like Example 3 above, this example confirms that SVV armed to carry a therapeutic protein which is functionally expressed in cells after transfection.

### Example 6: Construction of Additional Armed SVV Constructs

Using the general procedure shown in Example 1, additional SVV constructs were generated. Specifically, the following SVV armed constructs were designed: armed SVV encoding anti-CTLA4 nanobody; armed SVV encoding an anti-CD3 nanobody; armed SVV encoding an anti-PDL1 nanobody; armed SVV encoding both an anti-CTLA4+anti-PDL1 nanobody; armed SVV encoding both an anti-CTLA4+anti-CD3 nanobody; armed SVV encoding both an anti-CD3+anti-PDL1 nanobody; armed SVV encoding IL-2 (versions 2 and 3); armed SVV encoding TGF-beta dominant negative RII decoy-no SS v.2; armed SVV encoding TGF-beta dominant negative RII decoy-delta SerMet v.3; armed SVV encoding cytosine deaminase (FCY2+3); and armed SVV encoding Nfsa mut 22-78.

The protein and nucleic acid sequences of these inserts are shown in the Table 2 below.

TABLE 2
Therapeutic proteins encoded by armed SVV of Example 6

| SEQ | Description | Sequence |
|---|---|---|
| 29 | Nanobody | gctgctcacggtcacctgggtgccctggccccagtagtcgcccacgaactccatgc |
| | encoding anti- | cgtactggctgctgcagaagcccacggtggcgcagtagtagtcggcggtgtcctcg |
| | CTL-A | ggcttcaggctgttcatctgcaggaaggcggtgttcttggcgttgtcccggctgat |
| | nucleotide | ggtgaaccggcccttcacgaagtcggcgtagtaccgccggccctcgctgccgctgc |
| | sequence | tgatgcagctcacgccctcccgctccttgccgggggcctgccggaaccagccgatg |
| | | gcgtagttgtccaggctgaagccgctggcggcgcagctcagccgcaggctgccgcc |
| | | ggggtgcaccaggccgccgccgctctcctgcagctgcacctgaaattcgccgctgc |
| | | ccgccgcgctgcccgcgctgcccaggtgcagctgcaggagagcggcggcggcagcg |
| | | tgcaggccggcggcagcctgcggctgagctgcaccgccagcggcttcggcgtggac |
| | | ggcaccgacatgggctggtaccggcaggcccccggcaacgagtgcgagctggtgag |
| | | cagcatcagcagcatcggcatcggctactacagcgagagcgtgaagggccggttca |
| | | ccatcagccgggacaacgccaagaacaccgtgtacctgcagatgaacagcctgcgg |
| | | cccgacgacaccgccgtgtactactgcggccggcggtggatcggctaccggtgcgg |
| | | caactggggccggggcacccaggtgaccgtgagcagc |
| 30 | Nanobody | QVQLQESGGGLVHPGGSLRLSCAASGFSLDNYAIGWFRQAPGKEREGVSCISSGSE |
| | encoding anti- | GRRYYADFVKGRFTISRDNAKNTAFLQMNSLKPEDTADYYCATVGFCSSQYGMEFV |
| | CTL-A | GDYWGQGTQVTVSSGSAGSAAGSGEFQVQLQESGGGSVQAGGSLRLSCTASGFGVD |
| | protein | GTDMGWYRQAPGNECELVSSISSIGIGYYSESVKGRFTISRDNAKNTVYLQMNSLR |
| | sequence | PDDTAVYYCGRRWIGYRCGNWGRGTQVTVSS |
| 31 | Nanobody | GGAGGAGACGGTGACCTGGGTCCCCTGGCCCCAGTAGTCATTTGCACCCCATCCCC |
| | encoding anti- | CTTCACCCATTTTTGCACAGTAATACACGGCCGTGTCGTCAGGTTTCAGGCTGTTC |
| | CD3 | ATTTGCAGATACAGCGTGTTCTCGGCGTTGTCTCTGGAGATGGTGAACCGGCCCTT |
| | nucleotide | CACGGAGTCTCGATAGTATGTGCTACCACCATTCCAGCTAATATCTGAGACCCACT |
| | sequence | CCAGCCACTTCCCTGGAGCCTGTCGGACCCAGCTCATGCCATAATCATCAAAAGTG |
| | | AATCCAGAGGCTGCACAGGAGAGTCTCAGAGACCCCCCAGGCTGCACCAAGCCTCC |
| | | CCCAGACTCCACCAGCTGCACCTCCTCGAGCAGCTGCACCTC |
| 32 | Nanobody | EVQLLEEVQLVESGGGLVQPGGSLRLSCAASGFTEDDYGMSWVRQAPGKWLEWVSD |
| | encoding anti- | ISWNGGSTYYRDSVKGRFTISRDNAENTLYLQMNSLKPDDTAVYYCAKMGEGGWGA |
| | CD3 protein | NDYWGQGTQVTVSS |
| | sequence | |
| 33 | Nanobody | gctgctcacggtcacctgggtgccctggccccagtagtcgcccacgaactccatgc |
| | encoding anti- | cgtactggctgctgcagaagcccacggtggcgcagtagtagtcggcggtgtcctcg |
| | PDL1 | ggcttcaggctgttcatctgcaggaaggcggtgttcttggcgttgtcccggctgat |
| | nucleotide | ggtgaaccggcccttcacgaagtcggcgtagtaccgccggccctcgctgccgctgc |
| | sequence | tgatgcagctcacgccctcccgctccttgccgggggcctgccggaaccagccgatg |
| | | gcgtagttgtccaggctgaagccgctggcggcgcagctcagccgcaggctgccgcc |
| | | ggggtgcaccaggccgccgccgctctcctgcagctgcacctg |
| 34 | Nanobody | QVQLQESGGGLVHPGGSLRLSCAASGFSLDNYAIGWFRQAPGKEREGVSCISSGSE |
| | encoding anti- | GRRYYADFVKGRFTISRDNAKNTAFLQMNSLKPEDTADYYCATVGFCSSQYGMEFV |
| | PDL1 protein | GDYWGQGTQVTVSS |
| | sequence | |
| 35 | Nanobody | caggtgcagctgcaggagagcggcggcggcctggtgcaccccggcggcagcctgcg |
| | encoding anti- | gctgagctgcgccgccagcggcttcagcctggacaactacgccatcggctggttcc |
| | CTLA4+anti- | ggcaggcccccggcaaggagcgggagggcgtgagctgcatcagcagcggcagcgag |
| | PDL1 | ggccggcggtactacgccgacttcgtgaagggccggttcaccatcagccgggacaa |
| | nucleotide | cgccaagaacaccgccttcctgcagatgaacagcctgaagcccgaggacaccgccg |
| | sequence | actactactgcgccaccgtgggcttctgcagcagccagtacggcatggagttcgtg |
| | | ggcgactactggggccagggcacccaggtgaccgtgagcagcggcagcgcgggcag |
| | | cgcggcgggcagcggcgaatttcaggtgcagctgcaggagagcggcggcggcagcg |
| | | tgcaggccggcggcagcctgcggctgagctgcaccgccagcggcttcggcgtggac |
| | | ggcaccgacatgggctggtaccggcaggcccccggcaacgagtgcgagctggtgag |
| | | cagcatcagcagcatcggcatcggctactacagcgagagcgtgaagggccggttca |
| | | ccatcagccgggacaacgccaagaacaccgtgtacctgcagatgaacagcctgcgg |
| | | cccgacgacaccgccgtgtactactgcggccggcggtggatcggctaccggtgcgg |
| | | caactggggccggggcacccaggtgaccgtgagcagc |
| 36 | Nanobody | QVQLQESGGGLVHPGGSLRLSCAASGFSLDNYAIGWFRQAPGKEREGVSCISSGSE |
| | encoding anti- | GRRYYADFVKGRFTISRDNAKNTAFLQMNSLKPEDTADYYCATVGFCSSQYGMEFV |
| | CTLA4+anti- | GDYWGQGTQVTVSSGSAGSAAGSGEFQVQLQESGGGSVQAGGSLRLSCTASGFGVD |
| | PDL1 protein | GTDMGWYRQAPGNECELVSSISSIGIGYYSESVKGRFTISRDNAKNTVYLQMNSLR |
| | sequence | PDDTAVYYCGRRWIGYRCGNWGRGTQVTVSS |
| 37 | Nanobody | caggtgcagctgcaggagagcggcggcggcagcgtgcaggccggcggcagcctgcg |
| | encoding anti- | gctgagctgcaccgccagcggcttcggcgtggacggcaccgacatgggctggtacc |
| | CTLA4+anti- | ggcaggcccccggcaacgagtgcgagctggtgagcagcatcagcagcatcggcatc |
| | CD3 | ggctactacagcgagagcgtgaagggccggttcaccatcagccgggacaacgccaa |
| | nucleotide | gaacaccgtgtacctgcagatgaacagcctgcggcccgacgacaccgccgtgtact |
| | sequence | actgcggccggcggtggatcggctaccggtgcggcaactggggccggggcacccag |
| | | gtgaccgtgagcagcggcagcgcgggcagcgcggcgggcagcggcgaatttcaggt |
| | | gcagctgcaggagagcggcggcggcagcgtgcaggccggcggcagcctgcggctga |
| | | gctgcaccgccagcggcttcggcgtggacggcaccgacatgggctggtaccggcag |
| | | gcccccggcaacgagtgcgagctggtgagcagcatcagcagcatcggcatcggcta |
| | | ctacagcgagagcgtgaagggccggttcaccatcagccgggacaacgccaagaaca |
| | | ccgtgtacctgcagatgaacagcctgcggcccgacgacaccgccgtgtactactgc |
| | | ggccggcggtggatcggctaccggtgcggcaactggggccggggcacccaggtgac |
| | | cgtgagcagc |
| 38 | Nanobody | EVQLLEEVQLVESGGGLVQPGGSLRLSCAASGFTEDDYGMSWVRQAPGKWLEWVSD |
| | encoding anti- | ISWNGGSTYYRDSVKGRFTISRDNAENTLYLQMNSLKPDDTAVYYCAKMGEGGWGA |
| | CTLA4+anti- | NDYWGQGTQVTVSSGSAGSAAGSGEFQVQLQESGGGSVQAGGSLRLSCTASGFGVD |
| | CD3 protein | GTDMGWYRQAPGNECELVSSISSIGIGYYSESVKGRFTISRDNAKNTVYLQMNSLR |
| | sequence | PDDTAVYYCGRRWIGYRCGNWGRGTQVTVSS |
| 39 | Nanobody | caggtgcagctgcaggagagcggcggcggcctggtgcaccccggcggcagcctgcg |
| | encoding anti- | gctgagctgcgccgccagcggcttcagcctggacaactacgccatcggctggttcc |
| | anti-CD3 + | ggcaggcccccggcaaggagcgggagggcgtgagctgcatcagcagcggcagcgag |
| | anti-PLD1 | ggccggcggtactacgccgacttcgtgaagggccggttcaccatcagccgggacaa |
| | nucleotide | cgccaagaacaccgccttcctgcagatgaacagcctgaagcccgaggacaccgccg |
| | sequence | actactactgcgccaccgtgggcttctgcagcagccagtacggcatggagttcgtg |
| | | ggcgactactggggccagggcacccaggtgaccgtgagcagcggcagcgcgggcag |
| | | cgcggcgggcagcggcgaatttGAGGTGCAGCTGCTCGAGGAGGTGCAGCTGGTGG |
| | | AGTCTGGGGGAGGCTTGGTGCAGCCTGGGGGGTCTCTGAGACTCTCCTGTGCAGCC |
| | | TCTGGATTCACTTTTGATGATTATGGCATGAGCTGGGTCCGACAGGCTCCAGGGAA |
| | | GTGGCTGGAGTGGGTCTCAGATATTAGCTGGAATGGTGGTAGCACATACTATCGAG |
| | | ACTCCGTGAAGGGCCGGTTCACCATCTCCAGAGACAACGCCGAGAACACGCTGTAT |
| | | CTGCAAATGAACAGCCTGAAACCTGACGACACGGCCGTGTATTACTGTGCAAAAAT |
| | | GGGTGAAGGGGGATGGGGTGCAAATGACTACTGGGGCCAGGGGACCCAGGTCACCG |
| | | TCTCCTCC |
| 40 | Nanobody | QVQLQESGGGLVHPGGSLRLSCAASGFSLDNYAIGWFRQAPGKEREGVSCISSGSE |
| | encoding anti- | GRRYYADFVKGRFTISRDNAKNTAFLQMNSLKPEDTADYYCATVGFCSSQYGMEFV |
| | anti-CD3 + | GDYWGQGTQVTVSSGSAGSAAGSGEFEVQLLEEVQLVESGGGLVQPGGSLRLSCAA |
| | anti-PLD1 | SGFTFDDYGMSWVRQAPGKWLEWVSDISWNGGSTYYRDSVKGRFTISRDNAENTLY |
| | protein | LQMNSLKPDDTAVYYCAKMGEGGWGANDYWGQGTQVTVSS |
| | sequence | |
| 41 | IL-2 version 3 | gcgccgaccagcagcagcaccaaaaaaacccagctgcagctggaacatctgctgct |
| | nucleotide | ggatctgcagatgattctgaacggcattaacaactataaaaacccgaaactgaccc |
| | sequence | gcatgctgacctttaaattttatatgccgaaaaaagcgaccgaactgaaacatctg |
| | | cagtgcctggaagaagaactgaaaccgctggaagaagtgctgaacctggcgcagag |
| | | caaaaactttcatctgcgcccgcgcgatctgattagcaacattaacgtgattgtgc |
| | | tggaactgaaaggcagcgaaaccacctttatgtgcgaatatgcggatgaaaccgcg |
| | | accattgtggaatttctgaaccgctggattaccttttgccagagcattattagcac |
| | | cctgacc |
| 42 | IL-2 version 3 | APTSSSTKKTQLQLEHLLLDLQMILNGINNYKNPKLTRMLTFKFYMPKKATELKHL |
| | protein | QCLEEELKPLEEVLNLAQSKNFHLRPRDLISNINVIVLELKGSETTEMCEYADETA |
| | sequence | TIVEFLNRWITFCQSIISTLT |
| 43 | IL-2 version 2 | atgtatcgcatgcagctgctgagctgcattgcgctgagcctggcgctggtgaccaa |
| | nucleotide | cagcgcgccgaccagcagcagcaccaaaaaaacccagctgcagctggaacatctgc |
| | sequence | tgctggatctgcagatgattctgaacggcattaacaactataaaaacccgaaactg |
| | | acccgcatgctgacctttaaattttatatgccgaaaaaagcgaccgaactgaaaca |
| | | tctgcagtgcctggaagaagaactgaaaccgctggaagaagtgctgaacctggcgc |
| | | agagcaaaaactttcatctgcgcccgcgcgatctgattagcaacattaacgtgatt |
| | | gtgctggaactgaaaggcagcgaaaccacctttatgtgcgaatatgcggatgaaac |
| | | cgcgaccattgtggaatttctgaaccgctggattaccttttgccagagcattatta |
| | | gcaccctgacc |
| 44 | IL-2 version 2 | MYRMQLLSCIALSLALVTNSAPTSSSTKKTQLQLEHLLLDLQMILNGINNYKNPKL |
| | protein | TRMLTFKFYMPKKATELKHLQCLEEELKPLEEVLNLAQSKNFHLRPRDLISNINVI |
| | sequence | VLELKGSETTFMCEYADETATIVEFLNRWITFCQSIISTLT |
| 45 | TGF-beta | acgatcccaccgcacgttcagaagtcggttaataacgacatgatagtcactgacaa |
| | decoy version | caacggtgcagtcaagtttccacaactgtgtaaattttgtgatgtgagattttcca |
| | 2 nucleotide | cctgtgacaaccagaaatcctgcatgagcaactgcagcatcacctccatctgtgag |
| | sequence | aagccacaggaagtctgtgtggctgtatggagaaagaatgacgagaacataacact |
| | | agagacagtttgccatgaccccaagctcccctaccatgactttattctggaagatg |
| | | ctgcttctccaaagtgcattatgaaggaaaaaaaaaagcctggtgagactttcttc |
| | | atgtgttcctgtagctctgatgagtgcaatgacaacatcatcttctcagaagaata |
| | | taacaccagcaatcctgacttgttgctagtcatatttcaagtgacaggcatcagcc |
| | | tcctgccaccactgggagttgccatatctgtcatcatcatcttctactgctaccgc |
| | | gtttacccatacgatgttccagattacgct |
| 46 | TGF beta | TIPPHVQKSVNNDMIVTDNNGAVKFPQLCKFCDVRESTCDNQKSCMSNCSITSICE |
| | decoy version | KPQEVCVAVWRKNDENITLETVCHDPKLPYHDFILEDAASPKCIMKEKKKPGETFF |
| | 2 protein | MCSCSSDECNDNIIFSEEYNTSNPDLLLVIFQVTGISLLPPLGVAISVIIIFYCYR |
| | sequence | VYPYDVPDYA |
| 47 | TGF-beta | ggtcgggggctgctcaggggcctgtggccgctgcacatcgtcctgtggacgcgtat |
| | decoy version | cgccagcacgatcccaccgcacgttcagaagtcggttaataacgacatgatagtca |
| | 3 nucleotide | ctgacaacaacggtgcagtcaagtttccacaactgtgtaaattttgtgatgtgaga |
| | sequence | ttttccacctgtgacaaccagaaatcctgcatgagcaactgcagcatcacctccat |
| | | ctgtgagaagccacaggaagtctgtgtggctgtatggagaaagaatgacgagaaca |
| | | taacactagagacagtttgccatgaccccaagctcccctaccatgactttattctg |
| | | gaagatgctgcttctccaaagtgcattatgaaggaaaaaaaaaagcctggtgagac |
| | | tttcttcatgtgttcctgtagctctgatgagtgcaatgacaacatcatcttctcag |
| | | aagaatataacaccagcaatcctgacttgttgctagtcatatttcaagtgacaggc |
| | | atcagcctcctgccaccactgggagttgccatatctgtcatcatcatcttctactg |
| | | ctaccgcgtttacccatacgatgttccagattacgct |
| 48 | TGF beta | GRGLLRGLWPLHIVLWTRIASTIPPHVQKSVNNDMIVTDNNGAVKFPQLCKFCDVR |
| | decoy version | FSTCDNQKSCMSNCSITSICEKPQEVCVAVWRKNDENITLETVCHDPKLPYHDFIL |
| | 3 protein | EDAASPKCIMKEKKKPGETFFMCSCSSDECNDNIIFSEEYNTSNPDLLLVIFQVTG |
| | sequence | ISLLPPLGVAISVIIIFYCYRVYPYDVPDYA |
| 49 | Cytosine | ATGGTGACCGGCGGCATGGCCAGCAAGTGGGACCAGAAGGGCATGGACATCGCCTA |
| | deaminase | CGAGGAGGCCCTGCTGGGCTACAAGGAGGGCGGCGTGCCCATCGGCGGCTGCCTGA |
| | nucleotide | TCAACAACAAGGACGGCAGCGTGCTGGGCAGAGGCCACAACATGAGATTCCAGAAG |
| | sequence | GGCAGCGCCACCCTGCACGGCGAGATCAGCACCCTGGAGAACTGCGGCAGACTGGA |
| | | GGGCAAGGTGTACAAGGACACCACCCTGTACACCACCCTGAGCCCCTGCGACATGT |
| | | GCACCGGCGCCATCATCATGTACGGCATCCCCAGATGCGTGATCGGCGAGAACGTG |
| | | AACTTCAAGAGCAAGGGCGAGAAGTACCTGCAGACCAGAGGCCACGAGGTGGTGGT |
| | | GGTGGACGACGAGAGATGCAAGAAGCTGATGAAGCAGTTCATCGACGAGAGACCCC |
| | | AGGACTGGTTCGAGGACATCGGCGAG |
| 50 | Cytosine | MVTGGMASKWDQKGMDIAYEEALLGYKEGGVPIGGCLINNKDGSVLGRGHNMRFQK |
| | deaminase | GSATLHGEISTLENCGRLEGKVYKDTTLYTTLSPCDMCTGAIIMYGIPRCVIGENV |
| | protein | NFKSKGEKYLQTRGHEVVVVDDERCKKLMKQFIDERPQDWFEDIGE |
| | sequence | |
| 51 | Nfsa mut22- | atgaccccgaccattgaactgatttgcggccatcgcagcattcgccattttaccga |
| | 78 deaminase | tgaaccgattagcgaagcgcagcgcgaagcgattattaacagcgcgcgcgcgacca |
| | nucleotide | gcagcagctattttctgcagtgcagcagcattattcgcattaccgataaagcgctg |
| | sequence | cgcgaagaactggtgaccctgaccggcggccagaaacatgtggcgcaggcggcgga |
| | | attttgggtgttttgcgcggattttaaccgccatctgcagatttgcccggatgcgc |
| | | agctgggcctggcggaacagctgctgatgggctgggataccgcgatgatggcgcag |
| | | aacgcgctgattgcggcggaaagcctgggcctgggcggcgtgtatattggcggcct |
| | | gcgcaacaacattgaagcggtgaccaaactgctgaaactgccgcagcatgtgctgc |
| | | cgctgtttggcctgtgcctgggctggccggcggataacccggatctgaaaccgcgc |
| | | ctgccggcgagcattctggtgcatgaaaacagctatcagccgctggataaaggcgc |
| | | gctggcgcagtatgatgaacagctggcggaatattatctgacccgcggcagcaaca |
| | | accgccgcgatacctggagcgatcatattcgccgcaccattattGaagaaagcGCG |
| | | ccgTTTattctggattatctgcataaacagggctgggcgacccgc |
| 52 | Nfsa mut22- | MTPTIELICGHRSIRHFTDEPISEAQREAIINSARATSSSYFLQCSSIIRITDKAL |
| | 78 deaminase | REELVTLTGGQKHVAQAAEFWVFCADFNRHLQICPDAQLGLAEQLLMGWDTAMMAQ |
| | protein | NALIAAESLGLGGVYIGGLRNNIEAVTKLLKLPQHVLPLFGLCLGWPADNPDLKPR |
| | sequence | LPASILVHENSYQPLDKGALAQYDEQLAEYYLTRGSNNRRDTWSDHIRRTIIEESA |
| | | PFILDYLHKQGWATR |

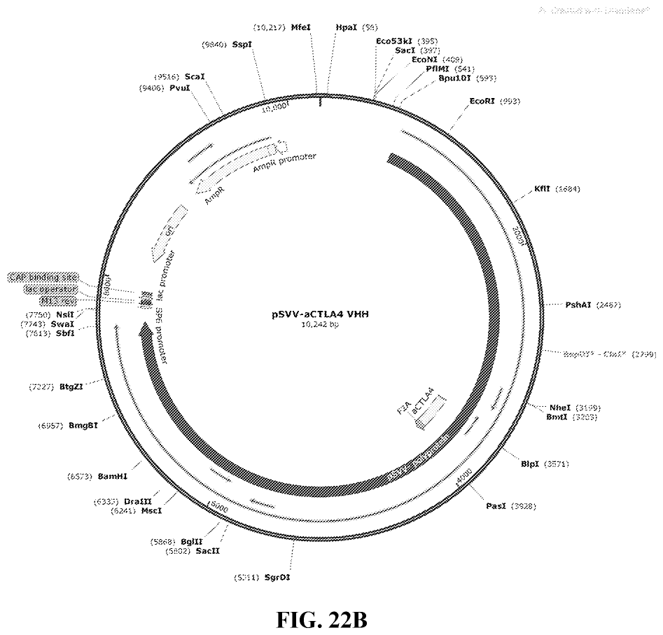

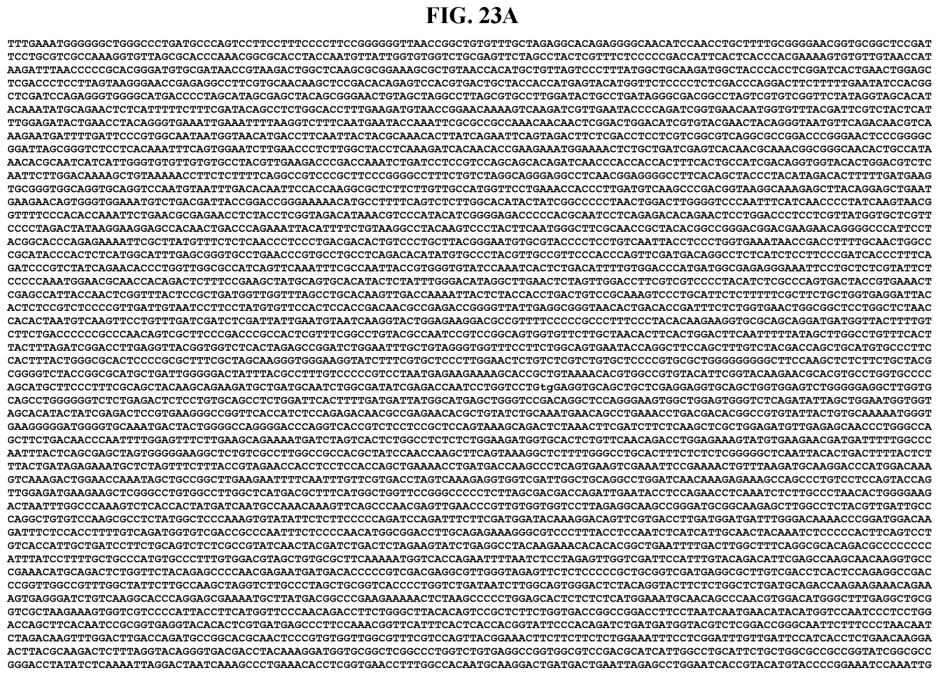

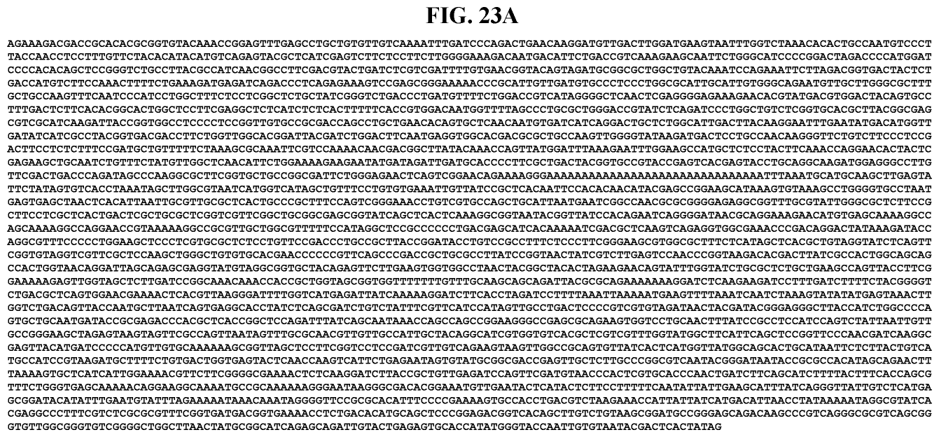

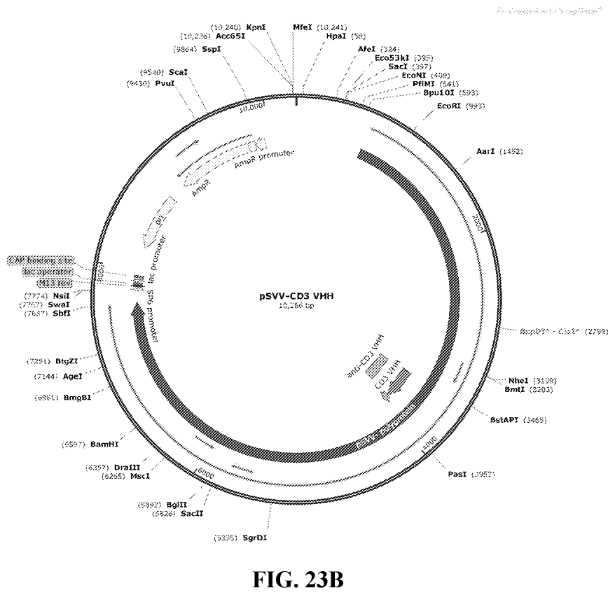

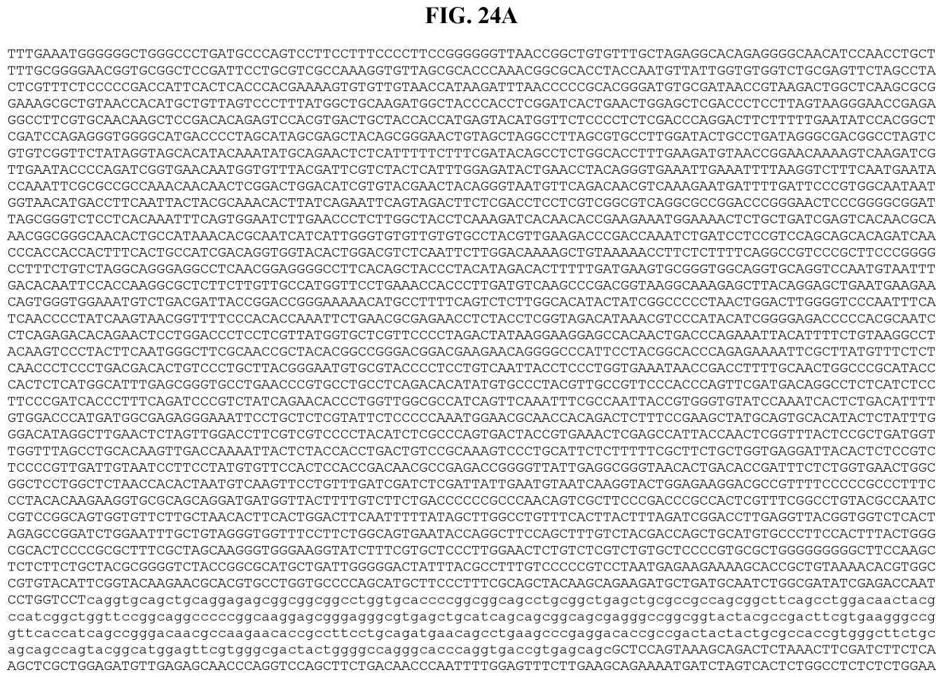

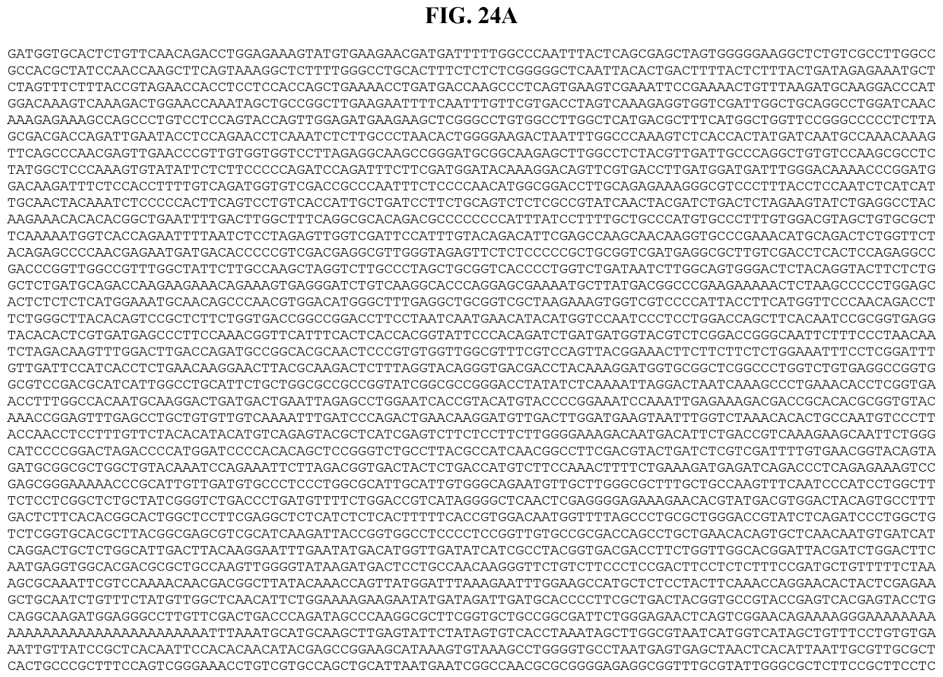

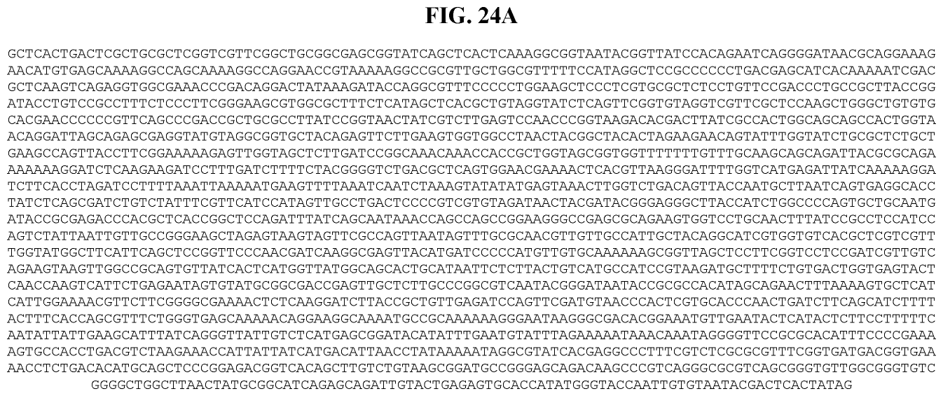

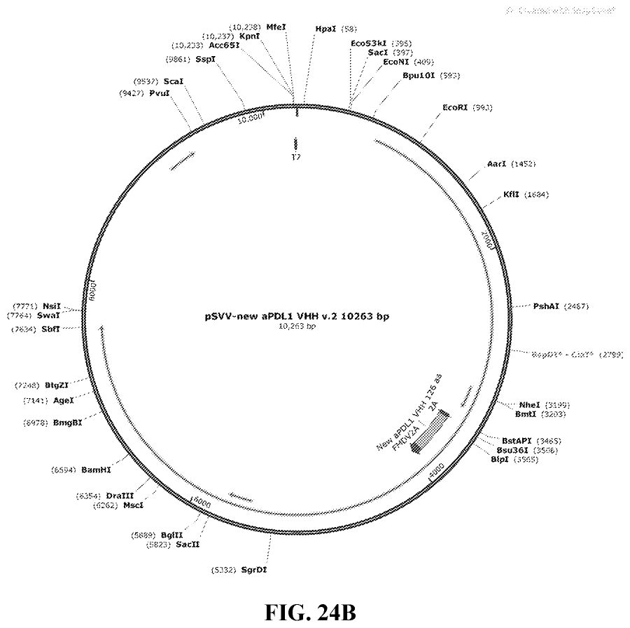

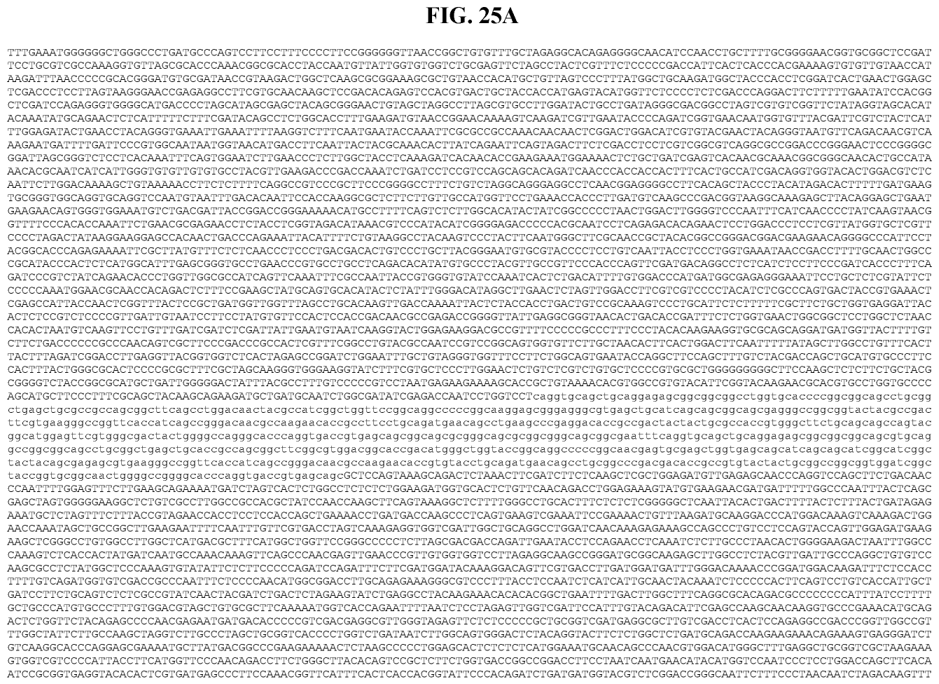

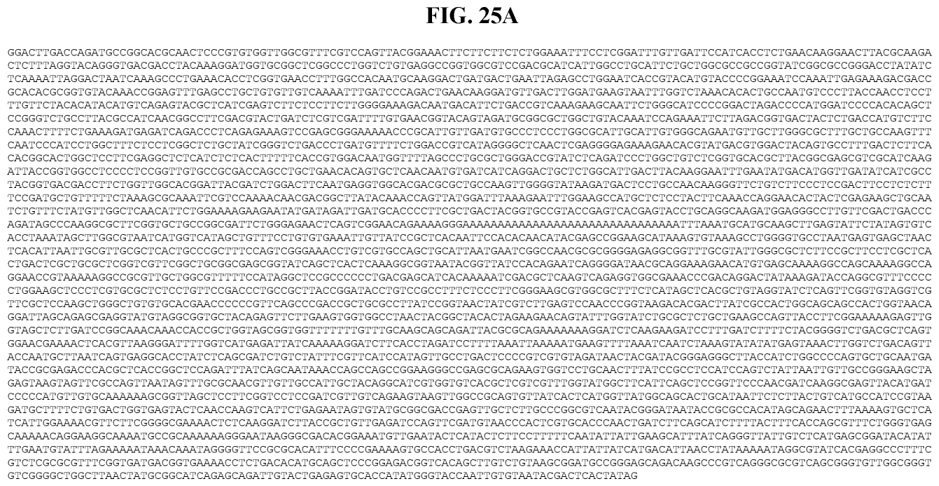

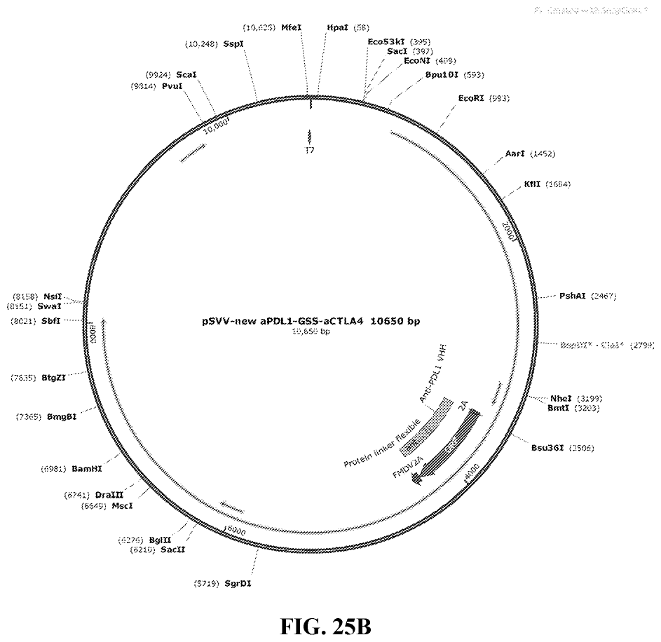

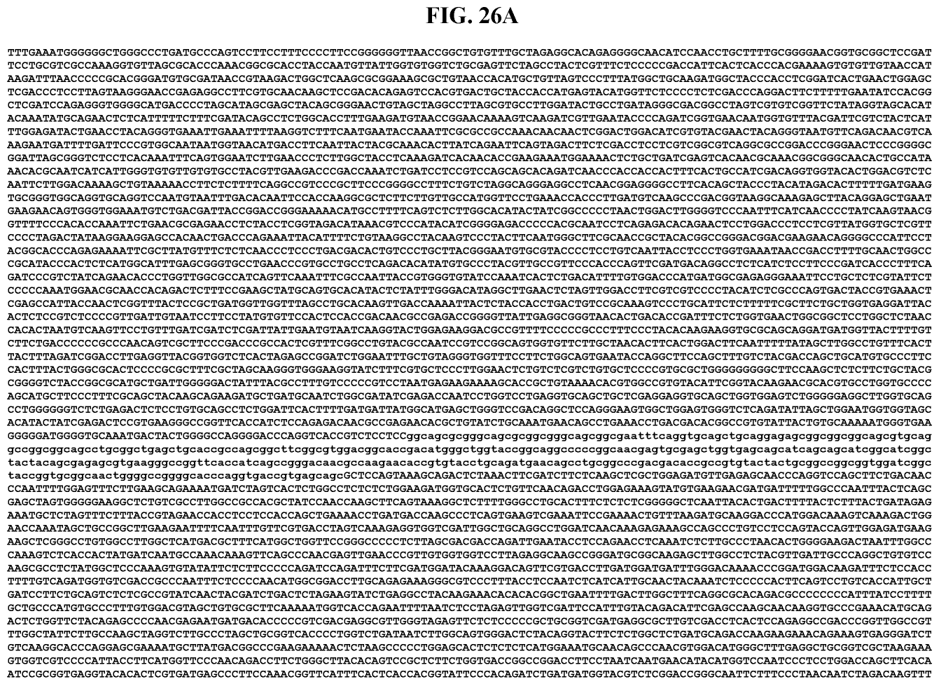

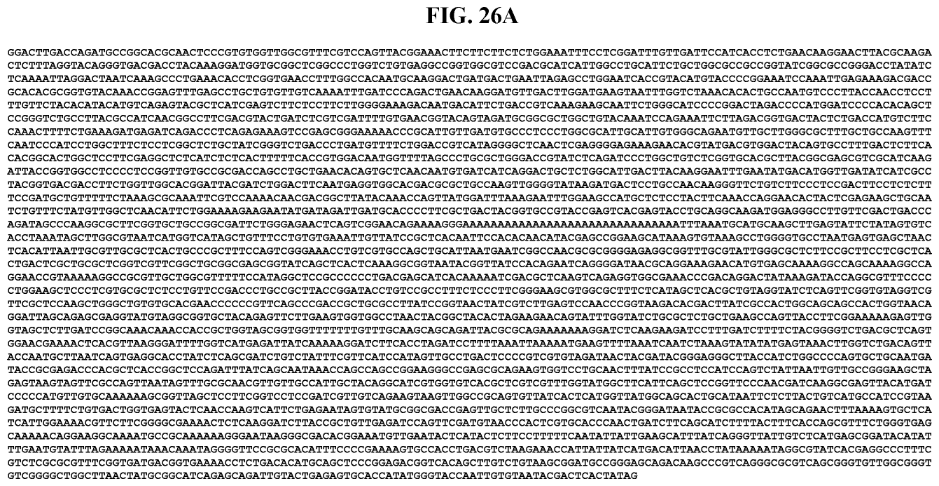

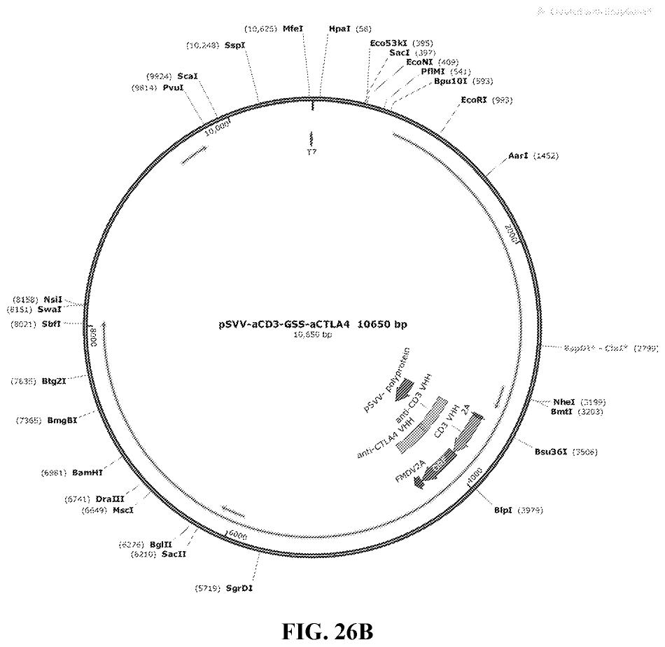

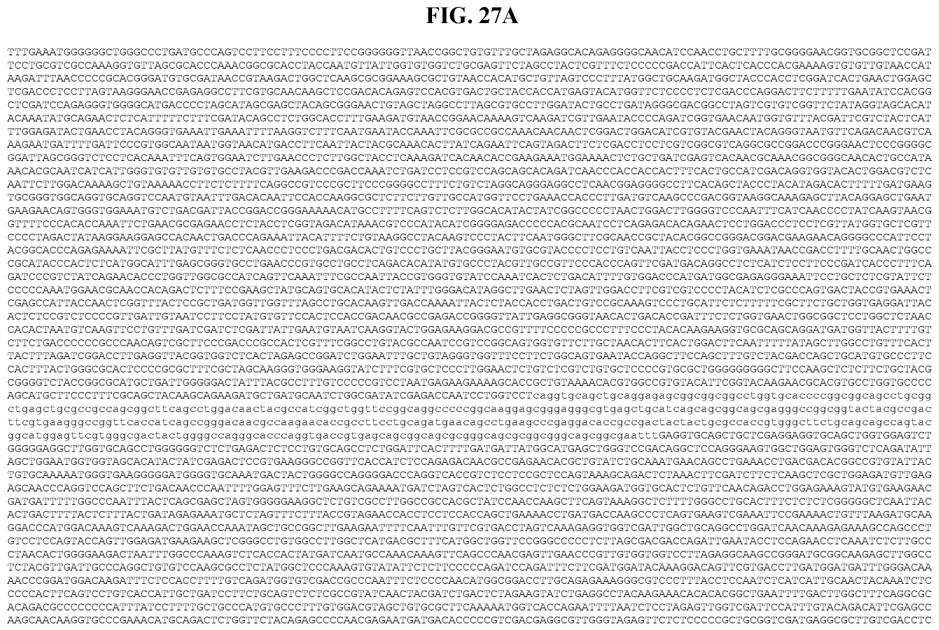

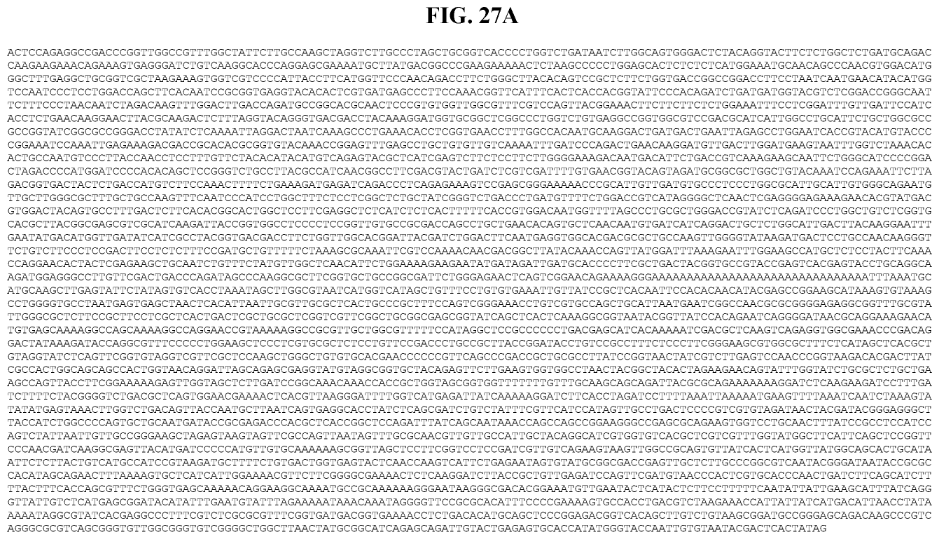

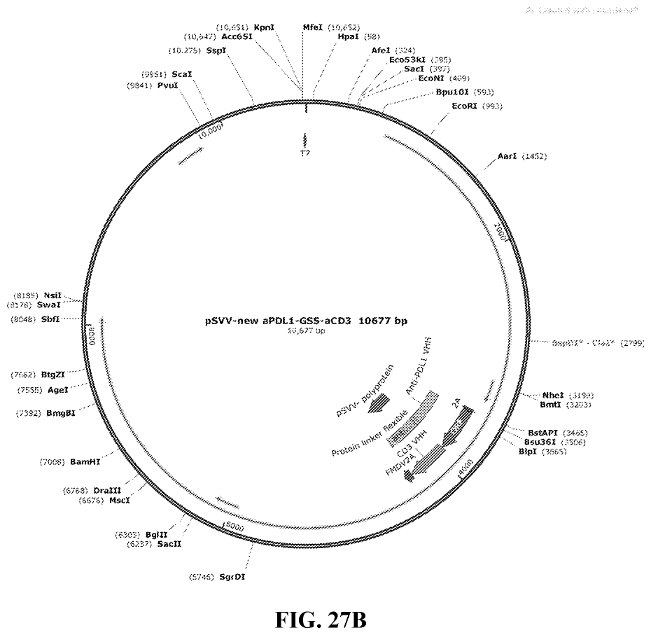

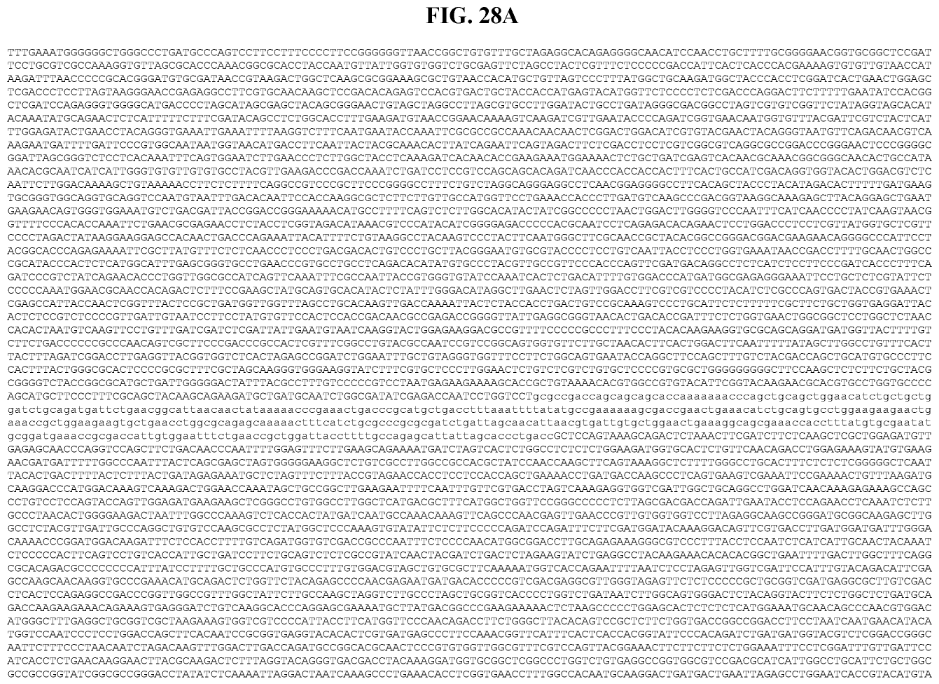

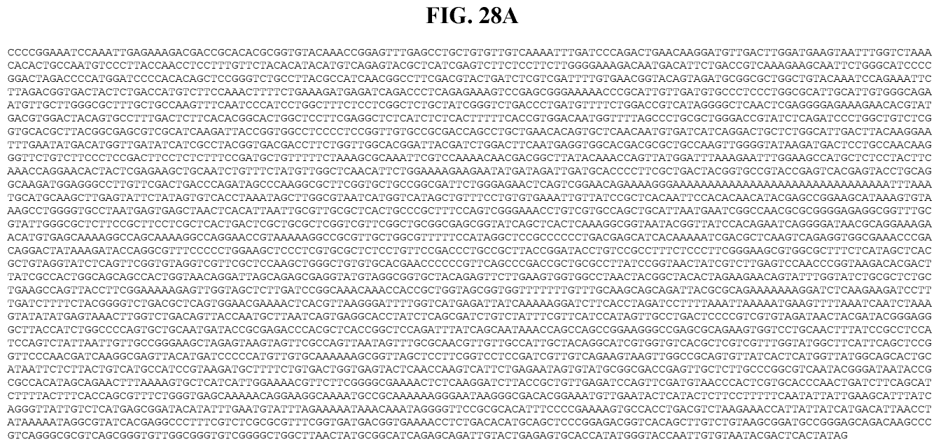

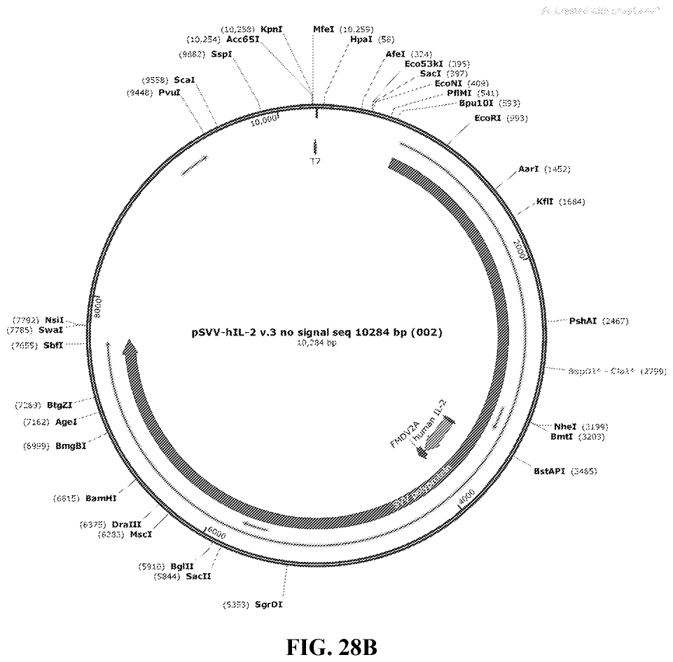

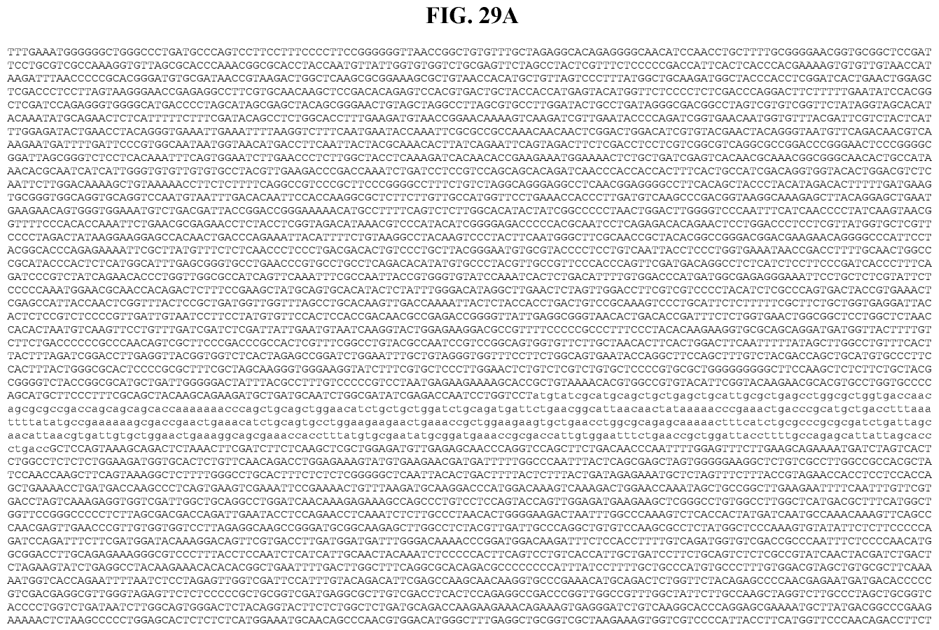

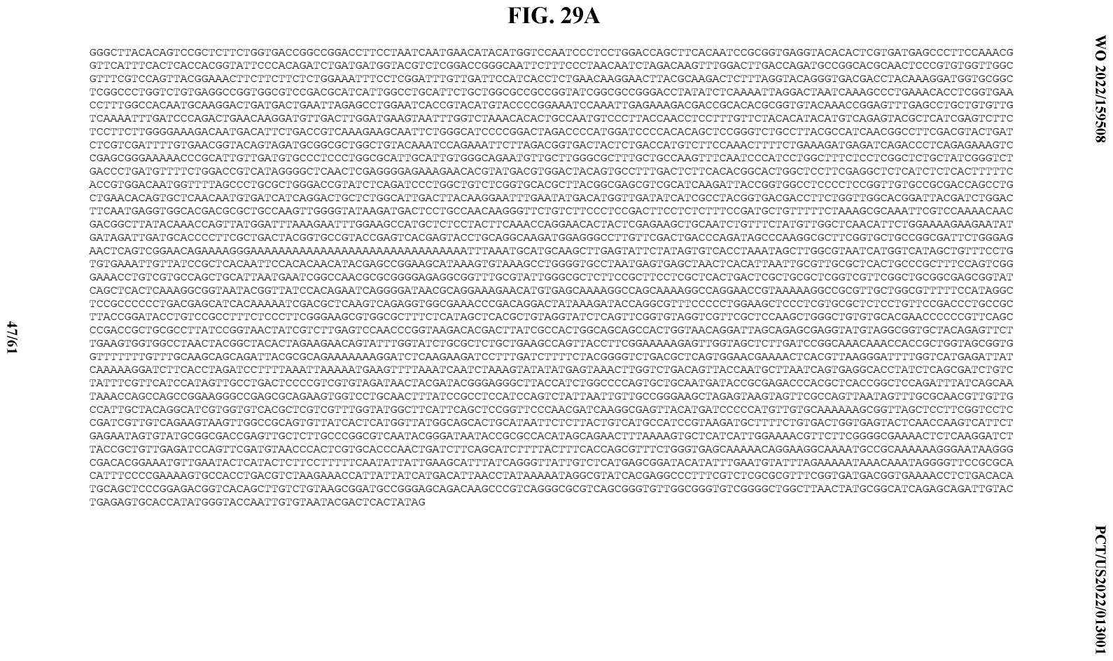

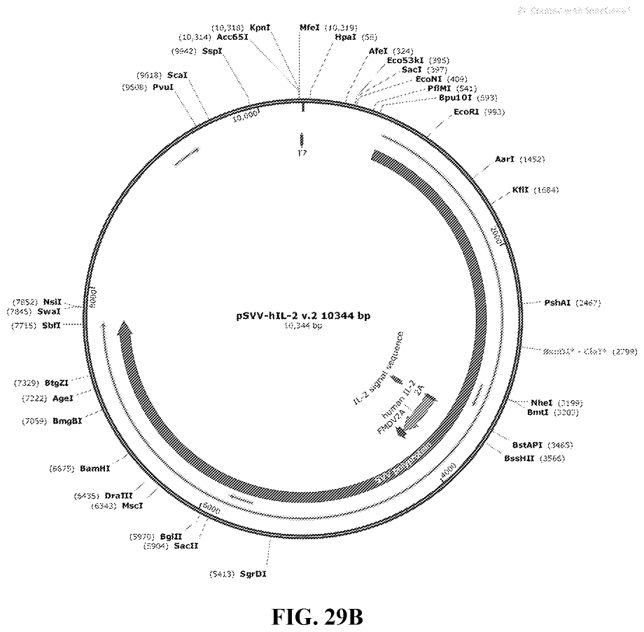

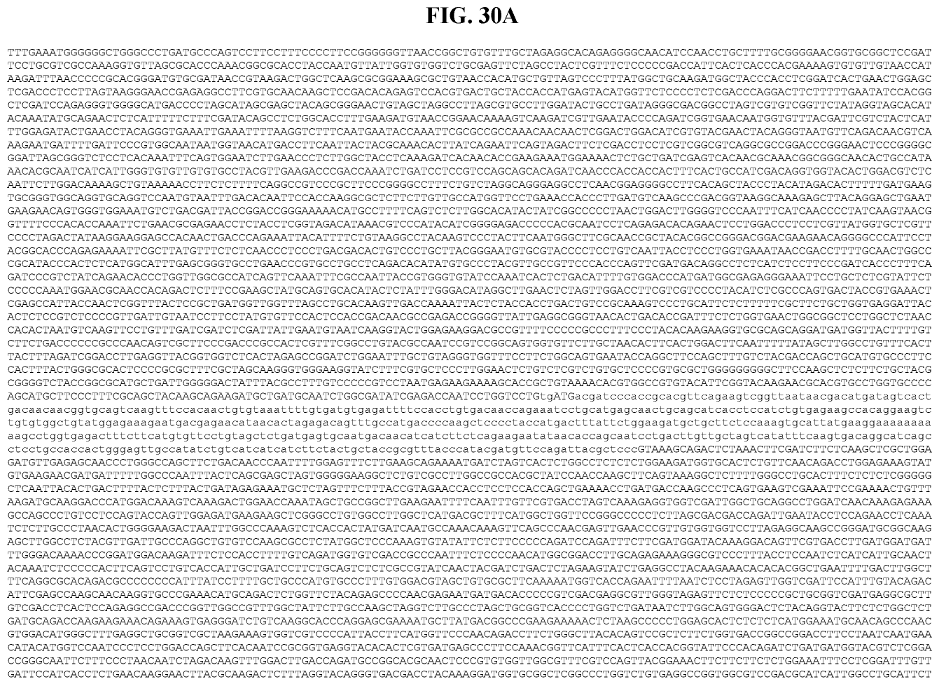

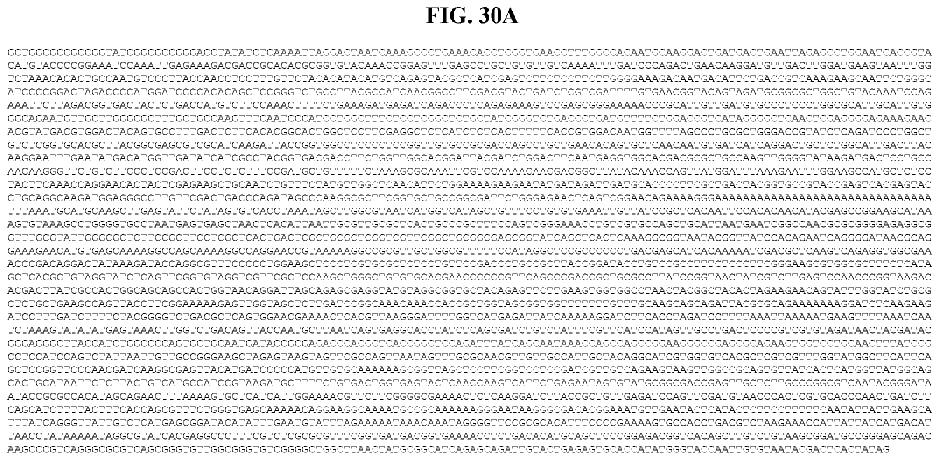

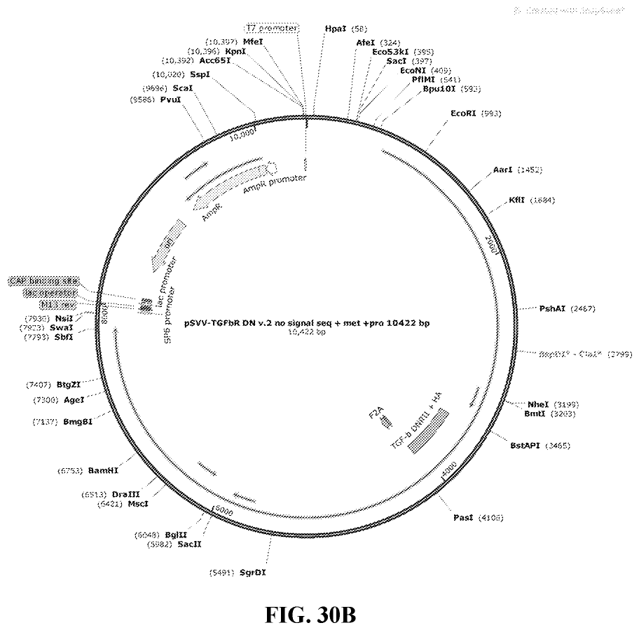

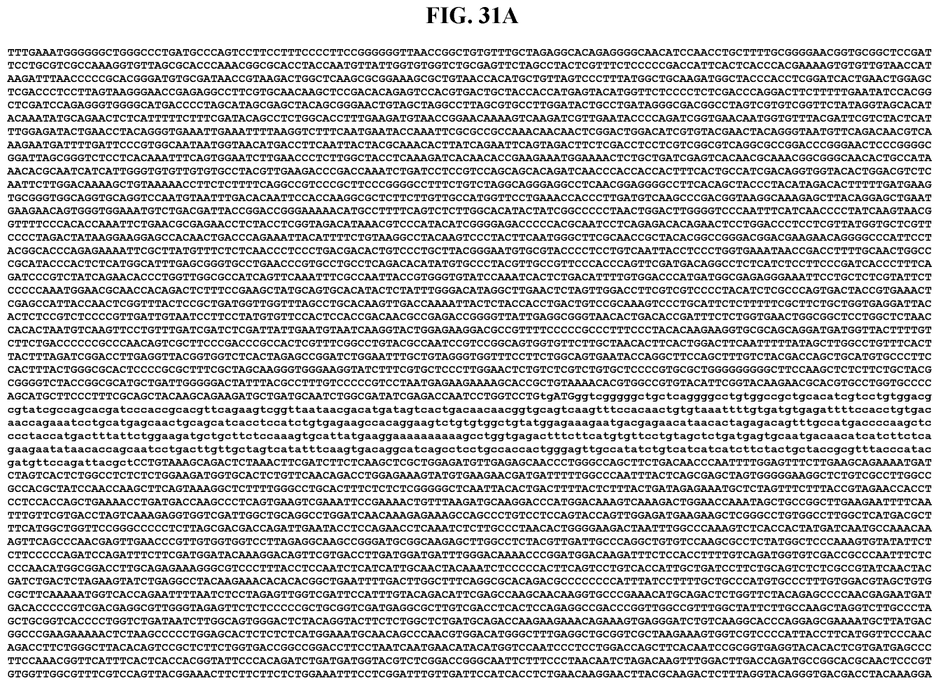

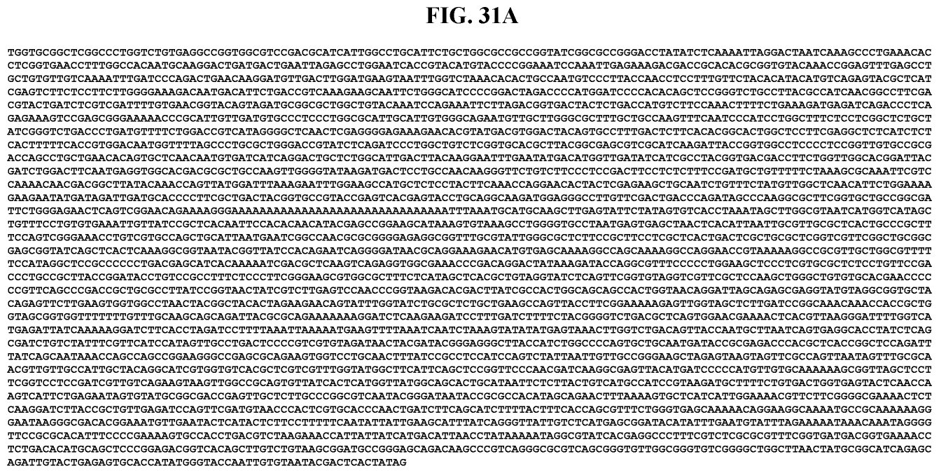

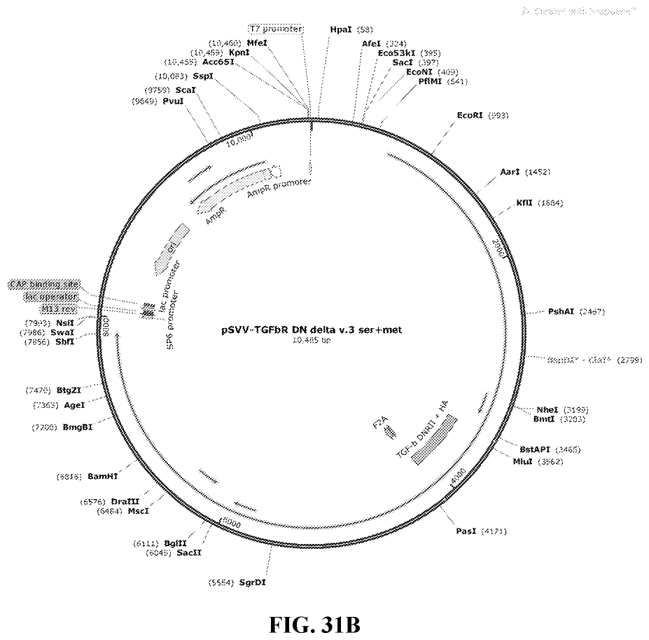

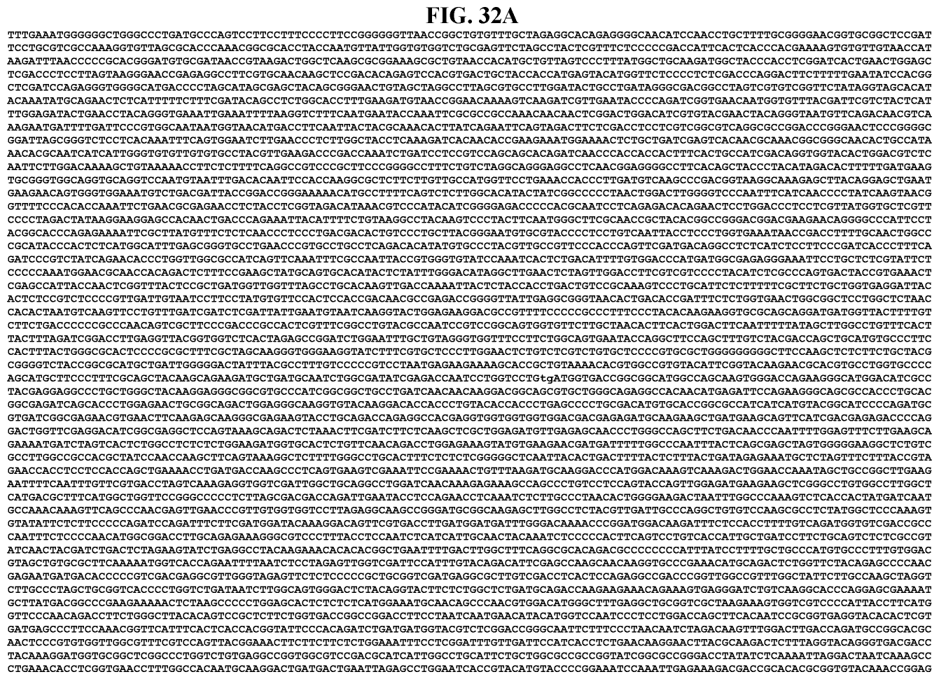

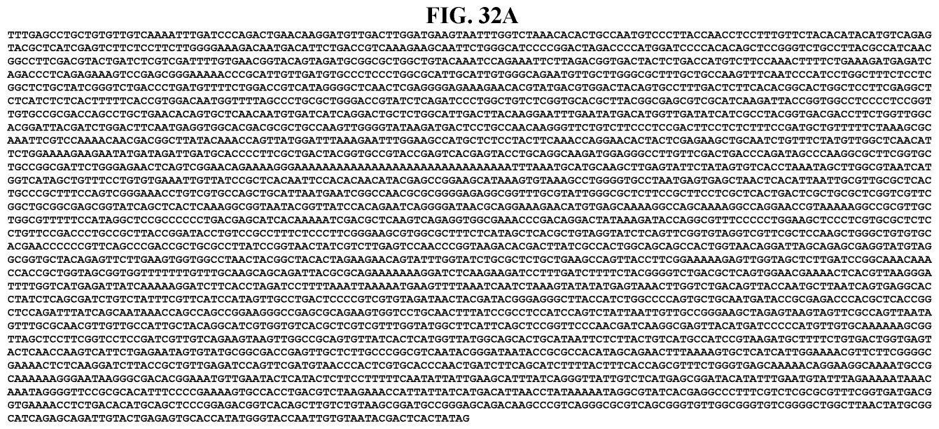

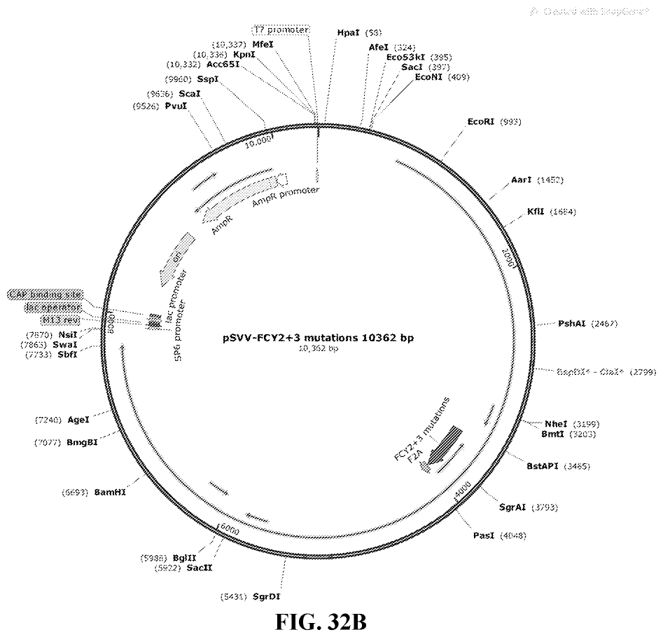

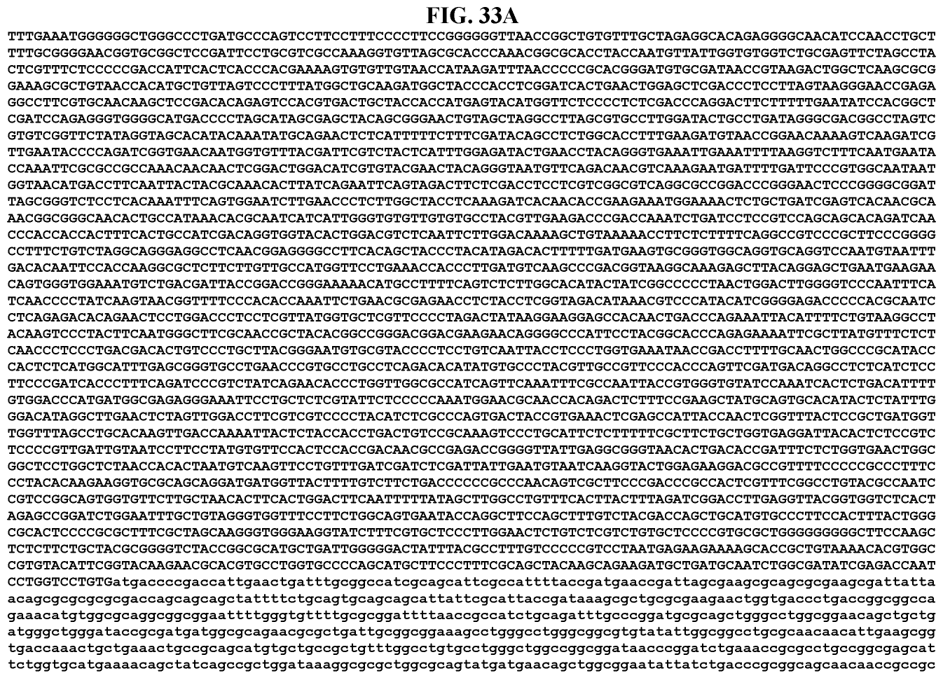

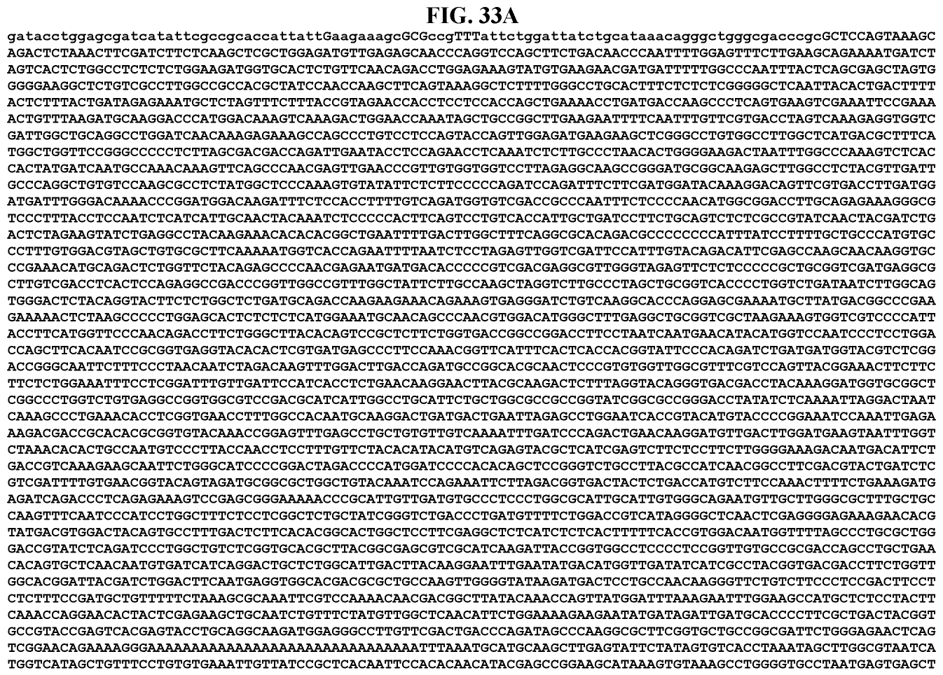

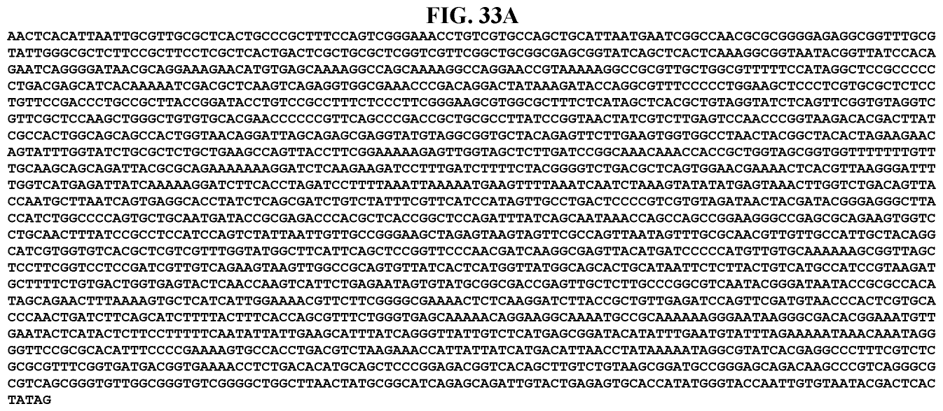

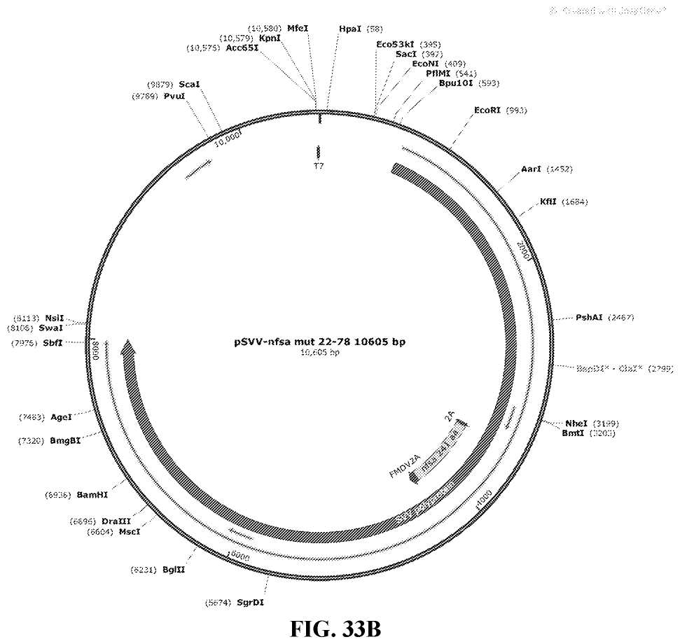

The sequences of the plasmids carrying these constructs (SEQ ID NO: 53-64) as well as the maps of these plasmids are shown in FIG. 22A-33B.

### ILLUSTRATIVE EMBODIMENTS

Provided here are illustrative embodiments of the disclosed technology. These embodiments are illustrative only and do not limit the scope of the present disclosure or of the claims attached.

Further Embodiment 1. An altered Seneca Valley Virus, wherein the altered Seneca Valley Virus comprises the sequence of a Seneca Valley Virus or oncolytic fragment thereof into which a nucleic acid has been inserted that is at least 85%, at least 90%, at least 95%, at least 99%, or 100% identical to nucleotides 1-7891 of SEQ ID NO: 19.

Further Embodiment 2. An armed Seneca Valley Virus, wherein the armed Seneca Valley Virus comprises the sequence of a Seneca Valley Virus or oncolytic fragment thereof into which a nucleic acid the nucleic acid sequence of SEQ ID NO: 1, 3, 5, 7, 9, or 11 has been inserted.

Further Embodiment 3. An armed Seneca Valley Virus, wherein the armed Seneca Valley Virus comprises the sequence of a Seneca Valley Virus or oncolytic fragment thereof into which a nucleic acid encoding the amino acid sequence of SEQ ID NO: 2, 4, 6, 8, 10, or 12 has been inserted.

Further Embodiment 4. An armed Seneca Valley Virus, wherein the armed Seneca Valley Virus comprises the sequence of a Seneca Valley Virus or oncolytic fragment thereof into which a nucleic acid encoding a protein at least 85%, at least 90%, at least 95%, or at 99% identical to the amino acid sequence of SEQ ID NO: 2, 4, 6, 8, 10, or 12 has been inserted.

Further Embodiment 5. An armed Seneca Valley Virus, wherein the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted.

Further Embodiment 6. An armed Seneca Valley Virus, wherein the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted.

Further Embodiment 7. An armed Seneca Valley Virus, wherein the armed Seneca Valley Virus comprises: a nucleotide sequence at least 85%, at least 90%, at least 95%, or at least 99% identical to nucleotides 1-7762 of SEQ ID NO: 13; a nucleotide sequence at least 85%, at least 90%, at least 95%, or at least 99% identical to nucleotides 1-7783 of SEQ ID NO: 14; a nucleotide sequence at least 85%, at least 90%, at least 95%, or at least 99% identical to nucleotides 1-7759 of SEQ ID NO: 15; a nucleotide sequence at least 85%, at least 90%, at least 95%, or at least 99% identical to nucleotides 1-7984 of SEQ ID NO: 16; a nucleotide sequence at least 85%, at least 90%, at least 95%, or at least 99% identical to nucleotides 1-8140 of SEQ ID NO: 17; or a nucleotide sequence at least 85%, at least 90%, at least 95%, or at least 99% identical to nucleotides 1-7738 of SEQ ID NO: 18.

Further Embodiment 8. An armed Seneca Valley Virus generated by inserting a nucleic acid sequence encoding a therapeutic protein into the genome of a Seneca Valley Virus between the coding sequences for protein 2A and 2B, wherein the nucleic acid encoding the therapeutic protein comprises: a nucleic acid of SEQ ID NO: 1, 3, 5, 7, 9, or 11; a nucleic acid at least 85%, 95%, or 99% identical to the nucleic acid sequence of SEQ ID NO: 1, 3, 5, 7, 9, or 11; a nucleic acid encoding the amino acid sequence of SEQ ID NO: 2, 4, 6, 8, 10, or 12; or a nucleic acid encoding a protein at least 85%, at least 90%, at least 95%, or at 99% identical to the amino acid sequence of SEQ ID NO: 2, 4, 6, 8, 10, or 12.

Further Embodiment 9. The armed Seneca Valley Virus of further embodiment 8, wherein the Seneca Valley Virus is SVV-001.

Further Embodiment 10. The armed Seneca Valley Virus of further embodiments 7 to 9, wherein the armed Seneca Valley Virus comprises: nucleotides 1-7762 of SEQ ID NO: 13; nucleotides 1-7783 of SEQ ID NO: 14; nucleotides 1-7759 of SEQ ID NO: 15; nucleotides 1-7984 of SEQ ID NO: 16; nucleotides 1-8140 of SEQ ID NO: 17; or nucleotides 1-7738 of SEQ ID NO: 18.

Further Embodiment 11. The armed Seneca Valley Virus of any one of further embodiments 1-10, wherein the armed Seneca Valley Virus is oncolytic and wherein the armed Seneca Valley Virus expresses a therapeutic agent or functional fragment thereof capable of treating cancer.

Further Embodiment 12. A vector comprising the armed Seneca Valley Virus of any one of further embodiments 1-11.

Further Embodiment 13. A plasmid comprising the nucleic acid sequence of SEQ ID NO: 13-18 or 53-64.

Further Embodiment 14. A plasmid comprising a nucleic acid at least 85%, at least 90%, at least 95% or at least 99% identical to the nucleic acid sequence of SEQ ID NO: 13-18 or 53-64.

Further Embodiment 15. A plasmid comprising a nucleic acid at least 85% or at least 90% identical to nucleotides 677-8050 in any of one the nucleic acid sequence of SEQ ID NO: 13-18 or 53-64.

Further Embodiment 16. A method of generating an armed Seneca Valley Virus comprising inserting a nucleic acid of SEQ ID NO: 1, 3, 5, 7, 9, or 11 or a nucleic acid encoding the amino acid sequence of SEQ ID NO: 2, 4, 6, 8, 10, or 12 in an Seneca Valley Virus.

Further Embodiment 17. A method of generating an armed Seneca Valley Virus comprising inserting a nucleic acid into an Seneca Valley Virus, wherein the nucleic acid is at least 85%, at least 90%, at least 95%, at least 99%, or 100% identical to: nucleotides 1-7762 of SEQ ID NO: 13; nucleotides 1-7783 of SEQ ID NO: 14; nucleotides 1-7759 of SEQ ID NO: 15; nucleotides 1-7984 of SEQ ID NO: 16; nucleotides 1-8140 of SEQ ID NO: 17; or nucleotides 1-7738 of SEQ ID NO: 18.

Further Embodiment 18. The method of further embodiments 16 or 17, wherein the nucleic acid is inserted into the genome of a Seneca Valley Virus between the coding sequences for protein 2A and 2B.

Further Embodiment 19. The method of any one of further embodiments 16 to 18, wherein the Seneca Valley Virus is SVV-001.

Further Embodiment 20. The method of any one of further embodiments 16 to 19, wherein the armed Seneca Valley Virus is oncolytic and wherein the armed Seneca Valley Virus expresses a therapeutic agent or functional fragment thereof capable of treating cancer.

Further Embodiment 21. A method of treating a cancer in a subject in need thereof comprising administering to the subject an effective amount of an armed Seneca Valley Virus of any one of further embodiments 1-11.

Further Embodiment 22. The method of further embodiment 21, wherein the subject is administered at least one anti-cancer therapeutic agent selected from the group consisting of: a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an IFN-I inhibiting agent, an engineered anti-cancer virus or virus derivative and a combination of any thereof.

Further Embodiment 23. The method of further embodiments 21 or 22, wherein the at least one anti-cancer therapeutic agent is administered formerly, simultaneously or subsequently to the administering of the armed Seneca Valley Virus.

Further Embodiment 24. The method of any one of further embodiments 21-23, wherein the subject is further administered at least one additional IFN-I inhibiting agent selected from the group consisting of: HDAC inhibitor, JAK/STAT inhibitor, IFN inhibitor, IFN antibody, IFN-α Receptor 1 antibody, IFN-α Receptor 2 antibody and viral peptide and a combination of any thereof.

Further Embodiment 25. The method of further embodiment 24, wherein the HDAC inhibitor is Trichostatin A.

Further Embodiment 26. The method of further embodiment 24, wherein the JAK/STAT inhibitor is staurosporine.

Further Embodiment 27. The method of any one of further embodiments 21-26, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Further Embodiment 28. The method of any one of further embodiments 21-227, wherein the success of cancer treatment is improved compared to treatment using Seneca Valley Virus that has not been armed.

Further Embodiment 29. A pharmaceutical composition for treating cancer in a subject in need thereof, the pharmaceutical composition comprising an armed Seneca Valley virus of any one of further embodiments 1-9 and a pharmaceutical acceptable carrier.

Further Embodiment 30. The pharmaceutical composition of further embodiment 29, wherein the composition further comprises a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an IFN-I inhibiting agent, an engineered anti-cancer virus or virus derivative and a combination of any thereof.

Further Embodiment 31. The pharmaceutical composition of further embodiments 29 or 30, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Further Embodiment 32. An armed Seneca Valley Virus of any one of further embodiments 1-11 for use in the manufacture of a medicament for treating cancer.

Further Embodiment 33. Use of an armed Seneca Valley Virus of any one of further embodiments 1-11 for treating a cancer.

Further Embodiment 34. The use of further embodiments 32 or 33, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Further Embodiment 35. The armed Seneca Valley Virus of any one of further embodiments 1-1 the vector of further embodiments 10 or 11, or the plasmid of any one of further embodiments 11 to 13, wherein the nucleic acid is RNA.

Further Embodiment 36. An altered Seneca Valley Virus comprising nucleotides 1-7891 of SEQ ID NO: 19.

Further Embodiment 37. An altered Seneca Valley Virus generated by inserting a nucleic acid sequence into the genome of a Seneca Valley Virus between the coding sequences for protein 2A and 2B, wherein the nucleic acid sequence comprise: nucleotides 3508-4014 of SEQ ID NO: 19; or a nucleic acid sequence that is at least 85%, at least 95%, or at least 99% identical to nucleotides 3508-4014 of SEQ ID NO: 19.

Further Embodiment 38. A vector or plasmid comprising nucleotides 1-7891 of SEQ ID NO: 19.

Alternate Embodiment 1. An altered Seneca Valley Virus, wherein the altered Seneca Valley Virus comprises the sequence of a Seneca Valley Virus or oncolytic fragment thereof into which a nucleic acid has been inserted that is at least 85%, at least 90%, at least 95%, at least 99%, or 100% identical to nucleotides 1-7891 of SEQ ID NO: 19.

Alternate Embodiment 2. A vector or plasmid encoding the altered Seneca Valley Virus of embodiment 1.

Alternate Embodiment 3. A plasmid comprising SEQ ID NO: 19 or a nucleotide that is at least 85%, at least 90%, at least 95%, at least 99%, or 100% identical to nucleotides 1-7891 of SEQ ID NO: 19.

Alternate Embodiment 4: The altered Seneca Valley Virus wherein the altered virus is oncolytic and expresses ova and Covid epitopes.

Alternate Embodiment 4. A method of generating an altered Seneca Valley Virus comprising inserting a nucleic acid into an Seneca Valley Virus, wherein the nucleic acid is at least 85%, at least 90%, at least 95%, at least 99%, or 100% identical to nucleotides 1-7891 of SEQ ID NO: 19.

Additional Embodiment 1. An armed Seneca Valley Virus, wherein the armed Seneca Valley Virus comprises Seneca Valley Virus or oncolytic fragment thereof and a nucleic acid encoding a therapeutic protein of interest.

Additional Embodiment 2. The armed Seneca Valley Virus of additional embodiment 1, wherein the protein of interest comprises an interleukin, a chemokine, or a nanobody acting as a checkpoint inhibitor.

Additional Embodiment 3. The armed Seneca Valley Virus of additional embodiment 2, wherein the therapeutic protein of interest comprises an anti-PD-L1 nanobody, IL-2 or mutant thereof, CXCL9, IL-15, IL-2/IL-15 (Neoleukin 2-15), a TGF-β decoy or mutant thereof, NfsA or mutant thereof, an anti-CTLA4 nanobody, an anti-CD3 nanobody, an anti-CTLA-4+anti-PDLI-1 nanobody, an anti-CLTA4+anti-PLD-1 nanobody, or a cytosine deaminase.

Additional Embodiment 4. The armed Seneca Valley Virus of any one of additional embodiments 1-3, wherein the therapeutic protein of interest comprises IL-2, CXCL-9, or IL-2/IL-15.

Additional Embodiment 5. The armed Seneca Valley Virus of any one additional embodiments 1-4, wherein the armed Seneca Valley Virus comprises Seneca Valley Virus or oncolytic fragment thereof into which the nucleic acid encoding a therapeutic protein of interest has been inserted.

Additional Embodiment 6. The armed Seneca Valley Virus of additional embodiment 5, wherein the armed Seneca Valley Virus comprises the sequence of a Seneca Valley Virus or oncolytic fragment thereof into which a nucleic acid the nucleic acid sequence of SEQ ID NO: 1, 3, 5, 7, 9, 11, 29, 31, 33, 35, 37, 39, 41, 43, 45, 47, 49, or 51 has been inserted.

Additional Embodiment 7. The armed Seneca Valley Virus of additional embodiment 5, wherein the armed Seneca Valley Virus comprises the sequence of a Seneca Valley Virus or oncolytic fragment thereof into which a nucleic acid encoding the amino acid sequence of SEQ ID NO: 2, 4, 6, 8, 10, 12, 30, 32, 34, 36, 38, 40, 42, 44, 46, 48, 50, or 52 has been inserted.

Additional Embodiment 8. The armed Seneca Valley Virus of additional embodiment 5, wherein the armed Seneca Valley Virus comprises the sequence of a Seneca Valley Virus or oncolytic fragment thereof into which a nucleic acid encoding a protein at least 85%, at least 90%, at least 95%, or at 99% identical to the amino acid sequence of SEQ ID NO: 2, 4, 6, 8, 10, 12, 30, 32, 34, 36, 38, 40, 42, 44, 46, 48, 50, or 52 has been inserted.

Additional Embodiment 9. The armed Seneca Valley Virus of additional embodiment 5, wherein the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted.

Additional Embodiment 10. The armed Seneca Valley Virus of additional embodiment 5, wherein the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted.

Additional Embodiment 11. The armed Seneca Valley Virus of additional embodiment 5, wherein the armed Seneca Valley Virus comprises: (a) a nucleotide sequence at least 85%, at least 90%, at least 95%, or at least 99% identical to nucleotides 1-7762 of SEQ ID NO: 13; (b) a nucleotide sequence at least 85%, at least 90%, at least 95%, or at least 99% identical to nucleotides 1-7783 of SEQ ID NO: 14; (c) a nucleotide sequence at least 85%, at least 90%, at least 95%, or at least 99% identical to nucleotides 1-7759 of SEQ ID NO: 15; (d) a nucleotide sequence at least 85%, at least 90%, at least 95%, or at least 99% identical to nucleotides 1-7984 of SEQ ID NO: 16; (e) a nucleotide sequence at least 85%, at least 90%, at least 95%, or at least 99% identical to nucleotides 1-8140 of SEQ ID NO: 17; or (f) a nucleotide sequence at least 85%, at least 90%, at least 95%, or at least 99% identical to nucleotides 1-7738 of SEQ ID NO: 18.

Additional Embodiment 12. An armed Seneca Valley Virus of additional embodiment 5, wherein the armed Seneca Valley Virus is generated by inserting a nucleic acid sequence encoding a therapeutic protein into the genome of a Seneca Valley Virus between the coding sequences for protein 2A and 2B, wherein the nucleic acid encoding the therapeutic protein comprises: (a) a nucleic acid of SEQ ID NO: 1, 3, 5, 7, 9, 11, 29, 31, 33, 35, 37, 39, 41, 43, 45, 47, 49, or 51; (b) a nucleic acid at least 85%, 95%, or 99% identical to the nucleic acid sequence of SEQ ID NO: 1, 3, 5, 7, 9, 11, 29, 31, 33, 35, 37, 39, 41, 43, 45, 47, 49, or 51; (c) a nucleic acid encoding the amino acid sequence of SEQ ID NO: 2, 4, 6, 8, 10, 12, 30, 32, 34, 36, 38, 40, 42, 44, 46, 48, 50, or 52; or (d) a nucleic acid encoding a protein at least 85%, at least 90%, at least 95%, or at 99% identical to the amino acid sequence of SEQ ID NO: 2, 4, 6, 8, 10, 12, 30, 32, 34, 36, 38, 40, 42, 44, 46, 48, 50, or 52.

Additional Embodiment 13. The armed Seneca Valley Virus of any one of additional embodiments 1-12, wherein the Seneca Valley Virus is SVV-001.

Additional Embodiment 14. The armed Seneca Valley Virus of additional embodiment 13, wherein the armed Seneca Valley Virus comprises: (a) nucleotides 1-7762 of SEQ ID NO: 13; (b) nucleotides 1-7783 of SEQ ID NO: 14; (c) nucleotides 1-7759 of SEQ ID NO: 15; (d) nucleotides 1-7984 of SEQ ID NO: 16; (e) nucleotides 1-8140 of SEQ ID NO: 17; or (f) nucleotides 1-7738 of SEQ ID NO: 18.

Additional Embodiment 15. An armed Seneca Valley Virus, wherein the armed Seneca Valley Virus comprises the sequence of a Seneca Valley Virus or oncolytic fragment thereof into which a nucleic acid has been inserted that is at least 85%, at least 90%, at least 95%, at least 99%, or 100% identical to nucleotides 1-7891 of SEQ ID NO: 19.

Additional Embodiment 16. The armed Seneca Valley Virus of any one of additional embodiments 1-15, wherein the armed Seneca Valley Virus is oncolytic and wherein the armed Seneca Valley Virus expresses a therapeutic agent or functional fragment thereof capable of treating cancer.

Additional Embodiment 17. A vector comprising the armed Seneca Valley Virus of any one of additional embodiments 1-16.

Additional Embodiment 18. A plasmid comprising the armed Seneca Valley Virus of any one of additional embodiments 1-16.

Additional Embodiment 19. The plasmid of additional embodiment 18, wherein the plasmid comprises the nucleic acid sequence of SEQ ID NO: 13-18 or 53-64.

Additional Embodiment 20. The plasmid of additional embodiment 18, wherein the plasmid comprises a nucleic acid at least 85%, at least 90%, at least 95% or at least 99% identical to the nucleic acid sequence of SEQ ID NO: 13-18 or 53-64.

Additional Embodiment 21. The plasmid of additional embodiment 18, wherein the plasmid comprises a nucleic acid at least 85% or at least 90% identical to nucleotides 677-8050 in any of one the nucleic acid sequence of SEQ ID NO: 13-18 or 53-64.

Additional Embodiment 22. A method of generating an armed Seneca Valley Virus comprising inserting a nucleic acid encoding a therapeutic protein of interest into a Seneca Valley Virus or oncolytic fragment thereof.

Additional Embodiment 23. The method of additional embodiment 22, wherein the protein of interest comprises an interleukin, a chemokine, or a nanobody acting as a checkpoint inhibitor.

Additional Embodiment 24. The method of additional embodiment 23, wherein the therapeutic protein of interest comprises an anti-PD-L1 nanobody, IL-2, CXCL9, IL-15, IL-2/IL-15, a TGF-β decoy, NfsA.

Additional Embodiment 25. The method of any one of additional embodiments 22-24, wherein the therapeutic protein of interest comprises IL-2, CXCL-9, or IL-2/IL-15.

Additional Embodiment 26. The method of any one of additional embodiments 22-26 wherein the method comprises: constructing a plasmid comprising the Seneca Valley Virus or oncolytic fragment thereof and the nucleic acid encoding a therapeutic protein of interest; linearizing the plasmid to define 3′ end; in vitro transcription reaction using T7 polymerase to generate RNA transcript with authentic 5′ and 3′ termini; transfection of the RNA transcript into target cells; and isolation of the armed SVV virus.

Additional Embodiment 27. The method of any one of additional embodiments 22-27 wherein the method comprises: cloning a T7 polymerase optimized mammalian expression plasmid into target cells; providing a linearized armed SVV plasmid comprising the Seneca Valley Virus or oncolytic fragment thereof and the nucleic acid encoding a therapeutic protein of interest; transfecting the armed SVV plasmid into the T7-pol target cells; and isolating the armed Seneca Valley Virus.

Additional Embodiment 28. The method of additional embodiment 27, further comprising constructing a plasmid comprising the Seneca Valley Virus or oncolytic fragment thereof and the nucleic acid encoding a therapeutic protein of interest.

Additional Embodiment 29. The method of additional embodiment 28, further comprising generating a linearized armed SVV plasmid.

Additional Embodiment 30. The method of any one of additional embodiments 22-29, wherein the nucleic acid is inserted into the genome of a Seneca Valley Virus between the coding sequences for protein 2A and 2B.

Additional Embodiment 31. The method of any one additional embodiments 22-30, wherein the Seneca Valley Virus is SVV-001.

Additional Embodiment 32. The method of any one of additional embodiments 22-31, wherein the armed Seneca Valley Virus is oncolytic and wherein the armed Seneca Valley Virus expresses a therapeutic agent or functional fragment thereof capable of treating cancer.

Additional Embodiment 33. The method of any one of additional embodiments 22-32, wherein the method comprises inserting a nucleic acid of SEQ ID NO: 1, 3, 5, 7, 9, 11, 29, 31, 33, 35, 37, 39, 41, 43, 45, 47, 49, or 51 or a nucleic acid encoding the amino acid sequence of SEQ ID NO: 2, 4, 6, 8, 10, 12, 30, 32, 34, 36, 38, 40, 42, 44, 46, 48, 50, or 52 into a Seneca Valley Virus or oncolytic fragment thereof.

Additional Embodiment 34. The method of any one of additional embodiments 22-32, wherein the method comprises inserting a nucleic acid into a Seneca Valley Virus or oncolytic fragment thereof, wherein the nucleic acid is at least 85%, at least 90%, at least 95%, at least 99%, or 100% identical to: nucleotides 1-7762 of SEQ ID NO: 13; nucleotides 1-7783 of SEQ ID NO: 14; nucleotides 1-7759 of SEQ ID NO: 15; nucleotides 1-7984 of SEQ ID NO: 16; nucleotides 1-8140 of SEQ ID NO: 17; or nucleotides 1-7738 of SEQ ID NO: 18.

Additional Embodiment 35. A method of treating a cancer in a subject in need thereof comprising administering to the subject an effective amount of an armed Seneca Valley Virus of any one of additional embodiments 1-16.

Additional Embodiment 36. The method of additional embodiment 35, wherein the subject is administered at least one anti-cancer therapeutic agent selected from the group consisting of a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an IFN-I inhibiting agent, an engineered anti-cancer virus or virus derivative and a combination of any thereof.

Additional Embodiment 37. The method of additional embodiments 35 or 36, wherein the at least one anti-cancer therapeutic agent is administered formerly, simultaneously or subsequently to the administering of the armed Seneca Valley Virus.

Additional Embodiment 38. The method of any one of additional embodiments 35-37, wherein the subject is further administered at least one additional IFN-I inhibiting agent selected from the group consisting of HDAC inhibitor, JAK/STAT inhibitor, IFN inhibitor, IFN antibody, IFN-α Receptor 1 antibody, IFN-α Receptor 2 antibody and viral peptide and a combination of any thereof.

Additional Embodiment 39. The method of any one of additional embodiments 35-38, wherein the HDAC inhibitor is Trichostatin A.

Additional Embodiment 40. The method of any one of additional embodiments 35-38, wherein the JAK/STAT inhibitor is staurosporine.

Additional Embodiment 41. The method of any one of additional embodiments 35-40, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Additional Embodiment 42. The method any one of additional embodiments 35-41, wherein the success of cancer treatment is improved compared to treatment using Seneca Valley Virus that has not been armed.

Additional Embodiment 43. A pharmaceutical composition for treating cancer in a subject in need thereof, the pharmaceutical composition comprising an armed Seneca Valley virus of any one of additional embodiments 1-10 and a pharmaceutical acceptable carrier.

Additional Embodiment 44. The pharmaceutical composition of additional embodiment 43, wherein the composition further comprises a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an IFN-I inhibiting agent, an engineered anti-cancer virus or virus derivative and a combination of any thereof.

Additional Embodiment 45. The pharmaceutical composition of additional embodiment 43, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Additional Embodiment 46. An armed Seneca Valley Virus of any one of additional embodiments 1-16 for use in the manufacture of a medicament for treating cancer.

Additional Embodiment 47. The use of additional embodiments 46, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Additional Embodiment 48. Use of an armed Seneca Valley Virus of any one of additional embodiments 1-10 for treating a cancer.

Additional Embodiment 49. The use of additional embodiment 48, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

Additional Embodiment 50. An armed Seneca Valley Virus comprising nucleotides 1-7891 of SEQ ID NO: 19.

Additional Embodiment 51. An armed Seneca Valley Virus generated by inserting a nucleic acid sequence into the genome of a Seneca Valley Virus between the coding sequences for protein 2A and 2B, wherein the nucleic acid sequence comprises: nucleotides 3508-4014 of SEQ ID NO: 19; or a nucleic acid sequence that is at least 85%, at least 95%, or at least 99% identical to nucleotides 3508-4014 of SEQ ID NO: 19.

Additional Embodiment 52. A vector or plasmid comprising nucleotides 1-7891 of SEQ ID NO: 19.

It is to be understood that while the disclosure has been described in conjunction with the preferred specific embodiments thereof, that the foregoing description and the examples that follow are intended to illustrate and not limit the scope of the disclosure.

It will be understood by those skilled in the art that various changes may be made, and equivalents may be substituted without departing from the scope of the disclosure, and further that other aspects, advantages and modifications will be apparent to those skilled in the art to which the disclosure pertains. In addition to the embodiments described herein, the present disclosure contemplates and claims those inventions resulting from the combination of features of the disclosure cited herein and those of the cited prior art references which complement the features of the present disclosure. Similarly, it will be appreciated that any described material, feature, or article may be used in combination with any other material, feature, or article, and such combinations are considered within the scope of this disclosure.

The disclosures of each patent, patent application, and publication cited or described herein are hereby incorporated herein by reference, each in its entirety, for all purposes.

1. An armed Seneca Valley Virus, wherein the armed Seneca Valley Virus comprises Seneca Valley Virus or oncolytic fragment thereof and a nucleic acid encoding a therapeutic protein of interest.

2. The armed Seneca Valley Virus of claim 1, wherein the protein of interest comprises an interleukin, a chemokine, or a nanobody acting as a checkpoint inhibitor.

3. The armed Seneca Valley Virus of claim 2, wherein the therapeutic protein of interest comprises an anti-PD-L1 nanobody, IL-2 or mutant thereof, CXCL9, IL-15, IL-2/IL-15 (Neoleukin 2-15), a TGF-β decoy or mutant thereof, NfsA or mutant thereof, an anti-CTLA4 nanobody, an anti-CD3 nanobody, an anti-CTLA-4+anti-PDLI-1 nanobody, an anti-CLTA4+anti-PLD-1 nanobody, or a cytosine deaminase.

4. The armed Seneca Valley Virus of claim 3, wherein the therapeutic protein of interest comprises IL-2, CXCL-9, or IL-2/IL-15.

5. The armed Seneca Valley Virus of claim 1, wherein the armed Seneca Valley Virus comprises Seneca Valley Virus or oncolytic fragment thereof into which the nucleic acid encoding a therapeutic protein of interest has been inserted.

6. The armed Seneca Valley Virus of claim 5, wherein the armed Seneca Valley Virus comprises the sequence of a Seneca Valley Virus or oncolytic fragment thereof into which a nucleic acid the nucleic acid sequence of SEQ ID NO: 1, 3, 5, 7, 9, 11, 29, 31, 33, 35, 37, 39, 41, 43, 45, 47, 49, or 51 has been inserted.

7. The armed Seneca Valley Virus of claim 5, wherein the armed Seneca Valley Virus comprises the sequence of a Seneca Valley Virus or oncolytic fragment thereof into which a nucleic acid encoding the amino acid sequence of SEQ ID NO: 2, 4, 6, 8, 10, 12, 30, 32, 34, 36, 38, 40, 42, 44, 46, 48, 50, or 52 has been inserted.

8. The armed Seneca Valley Virus of claim 5, wherein the armed Seneca Valley Virus comprises the sequence of a Seneca Valley Virus or oncolytic fragment thereof into which a nucleic acid encoding a protein at least 85%, at least 90%, at least 95%, or at 99% identical to the amino acid sequence of SEQ ID NO: 2, 4, 6, 8, 10, 12, 30, 32, 34, 36, 38, 40, 42, 44, 46, 48, 50, or 52 has been inserted.

9. The armed Seneca Valley Virus of claim 5, wherein the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted.

10. The armed Seneca Valley Virus of claim 5, wherein the armed Seneca Valley Virus comprises the sequence of a Seneca Valley Virus or oncolytic fragment thereof into which nucleotides 3508-3885 of SEQ ID NO: 13, nucleotides 3505-3906 of SEQ ID NO: 14, nucleotides 3508-3882 of SEQ ID NO: 15, nucleotides 3508-4107 of SEQ ID NO: 15, nucleotides 3508-4107 of SEQ ID NO: 16, nucleotides 3508-4263 of SEQ ID NO: 17, or nucleotides 3508-3861 of SEQ ID NO: 18 have been inserted.

11. The armed Seneca Valley Virus of claim 5, wherein the armed Seneca Valley Virus comprises:
(a) a nucleotide sequence at least 85%, at least 90%, at least 95%, or at least 99% identical to nucleotides 1-7762 of SEQ ID NO: 13;
(b) a nucleotide sequence at least 85%, at least 90%, at least 95%, or at least 99% identical to nucleotides 1-7783 of SEQ ID NO: 14;
(c) a nucleotide sequence at least 85%, at least 90%, at least 95%, or at least 99% identical to nucleotides 1-7759 of SEQ ID NO: 15;
(d) a nucleotide sequence at least 85%, at least 90%, at least 95%, or at least 99% identical to nucleotides 1-7984 of SEQ ID NO: 16;
Page 4 of 9
(e) a nucleotide sequence at least 85%, at least 90%, at least 95%, or at least 99% identical to nucleotides 1-8140 of SEQ ID NO: 17; or
(f) a nucleotide sequence at least 85%, at least 90%, at least 95%, or at least 99% identical to nucleotides 1-7738 of SEQ ID NO: 18.

12. The armed Seneca Valley Virus of claim 5, wherein the armed Seneca Valley Virus is generated by inserting a nucleic acid sequence encoding a therapeutic protein into the genome of a Seneca Valley Virus between the coding sequences for protein 2A and 2B, wherein the nucleic acid encoding the therapeutic protein comprises:
(a) a nucleic acid of SEQ ID NO: 1, 3, 5, 7, 9, 11, 29, 31, 33, 35, 37, 39, 41, 43, 45, 47, 49, or 51;
(b) a nucleic acid at least 85%, 95%, or 99% identical to the nucleic acid sequence of SEQ ID NO: 1, 3, 5, 7, 9, 11, 29, 31, 33, 35, 37, 39, 41, 43, 45, 47, 49, or 51;
(c) a nucleic acid encoding the amino acid sequence of SEQ ID NO: 2, 4, 6, 8, 10, 12, 30, 32, 34, 36, 38, 40, 42, 44, 46, 48, 50, or 52; or
(d) a nucleic acid encoding a protein at least 85%, at least 90%, at least 95%, or at 99% identical to the amino acid sequence of SEQ ID NO: 2, 4, 6, 8, 10, 12, 30, 32, 34, 36, 38, 40, 42, 44, 46, 48, 50, or 52.

13. The armed Seneca Valley Virus of claim 1, wherein the Seneca Valley Virus is SVV-001.

14. The armed Seneca Valley Virus of claim 13, wherein the armed Seneca Valley Virus comprises:
(a) nucleotides 1-7762 of SEQ ID NO: 13;
(b) nucleotides 1-7783 of SEQ ID NO: 14;
(c) nucleotides 1-7759 of SEQ ID NO: 15;
(d) nucleotides 1-7984 of SEQ ID NO: 16;
(e) nucleotides 1-8140 of SEQ ID NO: 17; or
(f) nucleotides 1-7738 of SEQ ID NO: 18.

15. An armed Seneca Valley Virus, wherein the armed Seneca Valley Virus comprises the sequence of a Seneca Valley Virus or oncolytic fragment thereof into which a nucleic acid has been inserted that is at least 85%, at least 90%, at least 95%, at least 99%, or 100% identical to nucleotides 1-7891 of SEQ ID NO: 19.

16. The armed Seneca Valley Virus of claim 1, wherein the armed Seneca Valley Virus is oncolytic and wherein the armed Seneca Valley Virus expresses a therapeutic agent or functional fragment thereof capable of treating cancer.

17. A vector comprising the armed Seneca Valley Virus of claim 1.

18. A plasmid comprising the armed Seneca Valley Virus of claim 1.

19. The plasmid of claim 18, wherein the plasmid comprises the nucleic acid sequence of SEQ ID NO: 13-18 or 53-64.

20. The plasmid of claim 18, wherein the plasmid comprises a nucleic acid at least 85%, at least 90%, at least 95% or at least 99% identical to the nucleic acid sequence of SEQ ID NO: 13-18 or 53-64.

21. The plasmid of claim 18, wherein the plasmid comprises a nucleic acid at least 85% or at least 90% identical to nucleotides 677-8050 in any of one the nucleic acid sequence of SEQ ID NO: 13-18 or 53-64.

22. A method of generating an armed Seneca Valley Virus comprising inserting a nucleic acid encoding a therapeutic protein of interest into a Seneca Valley Virus or oncolytic fragment thereof.

23. The method of claim 22, wherein the protein of interest comprises an interleukin, a chemokine, or a nanobody acting as a checkpoint inhibitor.

24.-34. (canceled)

35. A method of treating a cancer in a subject in need thereof comprising administering to the subject an effective amount of an armed Seneca Valley Virus of claim 1.

36. The method of claim 35, wherein the subject is administered at least one anti-cancer therapeutic agent selected from the group consisting of: a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an IFN-I inhibiting agent, an engineered anti-cancer virus or virus derivative and a combination of any thereof.

37. The method of claim 35, wherein the at least one anti-cancer therapeutic agent is administered formerly, simultaneously or subsequently to the administering of the armed Seneca Valley Virus.

38. The method of claim 35, wherein the subject is further administered at least one additional IFN-I inhibiting agent selected from the group consisting of HDAC inhibitor, JAK/STAT inhibitor, IFN inhibitor, IFN antibody, IFN-α Receptor 1 antibody, IFN-α Receptor 2 antibody and viral peptide and a combination of any thereof.

39. The method of claim 35, wherein the HDAC inhibitor is Trichostatin A.

40. The method of claim 35, wherein the JAK/STAT inhibitor is staurosporine.

41. The method of claim 35, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

42. The method of claim 35, wherein the success of cancer treatment is improved compared to treatment using Seneca Valley Virus that has not been armed.

43. A pharmaceutical composition for treating cancer in a subject in need thereof, the pharmaceutical composition comprising an armed Seneca Valley virus of claim 1 and a pharmaceutical acceptable carrier.

44. The pharmaceutical composition of claim 43, wherein the composition further comprises a checkpoint inhibitor, a PD-1 inhibitor, a PD-L1 inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a CAR therapy, a dendritic cell-based therapy, a cancer vaccine, an oncolytic virus, an IFN-I inhibiting agent, an engineered anti-cancer virus or virus derivative and a combination of any thereof.

45. The pharmaceutical composition of claim 43, wherein the cancer comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer.

46.-49. (canceled)

50. An armed Seneca Valley Virus comprising nucleotides 1-7891 of SEQ ID NO: 19.

51. (canceled)

52. A vector or plasmid comprising nucleotides 1-7891 of SEQ ID NO: 19.


SEQUENCE LISTING (52 sequences)

SEQ ID NO: 1 - DNA, 381 bp, Homo sapiens
caggtgcagctgcaggagtctggaggaggctcggtgcagaccggagggtctctgagactctcctgtacagcctctacatcaatatacagtaacaactacatggcctggttcagccagtctccaggaaaggggcgcgagggggtcgcagctgtttatatggatgatggtcgcccatactatgccgattccgtgaagggccgattcaccatctccctagacagcgccaagaacacgatgtatttgcaaatgaacagcctgaaacctgaggacactgccatgtactactgtgcggcagctccaggccccttaagtcgtaactactggtacacgtccgccaactatgactactggggccaggggacccaggtcaccgtctcctca

SEQ ID NO: 2 - protein, 127 aa, Homo sapiens
QVQLQESGGGSVQTGGSLRLSCTASTSIYSNNYMAWFSQSPGKGREGVAAVYMDDGRPYYADSVKGRFTISLDSAKNTMYLQMNSLKPEDTAMYYCAAAPGPLSRNYWYTSANYDYWGQGTQVTVSS

SEQ ID NO: 3 - DNA, 399 bp, Homo sapiens
gctcctgcctcctccagcaccaagaaaacccagctccagctggaacatctcctgctggatctgcagatgatcctgaacggcatcaacaactacaagaaccccaagctgacccggatgctgaccgccaagttcgccatgcccaagaaggccaccgagctgaaacatctgcagtgcctggaagaggaactgaagcctctggaagaggtgctgaacggcgcccagtccaagaacttccacctgaggcctcgggacctgatctccaacatcaacgtgatcgtgctggaactgaagggctccgagacaaccttcatgtgcgagtacgccgacgagacagctaccatcgtggaatttctgaaccggtggatcaccttcgcccagtccatcatctccaccctgacc

SEQ ID NO: 4 - protein, 133 aa, Homo sapiens
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 5 - DNA, 378 bp, Homo sapiens
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 6 - protein, 124 aa, Homo sapiens
MKKSGVLFLLGIILLVLIGVQGTPVVRKGRCSCISTNQGTHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 7 - DNA, 600 bp, Homo sapiens
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtttacccatacgatgttccagattacgct

SEQ ID NO: 8 - protein, 200 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVYPYDVPDYA

SEQ ID NO: 9 - DNA, 756 bp, Homo sapiens
agcgcgatgccgctggaaagcgaacaggaaaactgcgaaatgaccccgaccattgaactgatttgcggccatcgcagcattcgccattttaccgatgaaccgattagcgaagcgcagcgcgaagcgattattaacagcgcgcgcgcgaccagcagcagctattttctgcagtgcagcagcattattcgcattaccgataaagcgctgcgcgaagaactggtgaccctgaccggcggccagaaacatgtggcgcaggcggcggaattttgggtgttttgcgcggattttaaccgccatctgcagatttgcccggatgcgcagctgggcctggcggaacagctgctgatgggctgggataccgcgatgatggcgcagaacgcgctgattgcggcggaaagcctgggcctgggcggcgtgtatattggcggcctgcgcaacaacattgaagcggtgaccaaactgctgaaactgccgcagcatgtgctgccgctgtttggcctgtgcctgggctggccggcggataacccggatctgaaaccgcgcctgccggcgagcattctggtgcatgaaaacagctatcagccgctggataaaggcgcgctggcgcagtatgatgaacagctggcggaatattatctgacccgcggcagcaacaaccgccgcgatacctggagcgatcatattcgccgcaccattattaaagaaagccgcccgagcattctggattatctgcataaacagggctgggcgacccgc

SEQ ID NO: 10 - protein, 252 aa, Homo sapiens
SAMPLESEQENCEMTPTIELICGHRSIRHFTDEPISEAQREAIINSARATSSSYFLQCSSIIRITDKALREELVTLTGGQKHVAQAAEFWVFCADFNRHLQICPDAQLGLAEQLLMGWDTAMMAQNALIAAESLGLGGVYIGGLRNNIEAVTKLLKLPQHVLPLFGLCLGWPADNPDLKPRLPASILVHENSYQPLDKGALAQYDEQLAEYYLTRGSNNRRDTWSDHIRRTIIKESRPSILDYLHKQGWATR

SEQ ID NO: 11 - DNA, 354 bp, Homo sapiens
atgaagtgggtgaccttcatcagcctgctgttcctgttcagcagcgcctacagccccaagaagaagatccagctgcacgccgagcacgccctgtacgacgccctgatgatcctgaacatcgtgaagaccaacagcccccccgccgaggagaagctggaggactacgccttcaacttcgagctgatcctggaggagatcgccagactgttcgagagcggcgaccagaaggacgaggccgagaaggccaagagaatgaaggagtggatgaagagaatcaagaccaccgccagcgaggacgagcaggaggagatggccaacgccatcatcaccatcctgcagagctggatcttcagc

SEQ ID NO: 12 - protein, 118 aa, Homo sapiens
MKWVTFISLLFLFSSAYSPKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 13 - DNA, 10263 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctcaggtgcagctgcaggagagcggcggcggcagcgtgcagaccggcggcagcctgagactgagctgcaccgccagcaccagcatctacagcaacaactacatggcctggttcagccagcccggcaagggcagagagggcgtggccgccgtgtacatggacgacggcagaccctactacgccgacagcgtgaagggcagattcaccatcagcctggacagcgccaagaacaccatgtacctgcagatgaacagcctgaagcccgaggacaccgccatgtactactgcgccgccgcccccggccccctgagcagaaactactggtacaccagcgccaactacgactactggggccagggcacccaggtgaccgtgagcagcgctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 14 - DNA, 10284 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctgctcctgcctcctccagcaccaagaaaacccagctccagctggaacatctcctgctggatctgcagatgatcctgaacggcatcaacaactacaagaaccccaagctgacccggatgctgaccgccaagttcgccatgcccaagaaggccaccgagctgaaacatctgcagtgcctggaagaggaactgaagcctctggaagaggtgctgaacggcgcccagtccaagaacttccacctgaggcctcgggacctgatctccaacatcaacgtgatcgtgctggaactgaagggctccgagacaaccttcatgtgcgagtacgccgacgagacagctaccatcgtggaatttctgaaccggtggatcaccttcgcccagtccatcatctccaccctgaccgctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 15 - DNA, 10260 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacagctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 16 - DNA, 10485 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtttacccatacgatgttccagattacgctgctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 17 - DNA, 10641 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctagcgcgatgccgctggaaagcgaacaggaaaactgcgaaatgaccccgaccattgaactgatttgcggccatcgcagcattcgccattttaccgatgaaccgattagcgaagcgcagcgcgaagcgattattaacagcgcgcgcgcgaccagcagcagctattttctgcagtgcagcagcattattcgcattaccgataaagcgctgcgcgaagaactggtgaccctgaccggcggccagaaacatgtggcgcaggcggcggaattttgggtgttttgcgcggattttaaccgccatctgcagatttgcccggatgcgcagctgggcctggcggaacagctgctgatgggctgggataccgcgatgatggcgcagaacgcgctgattgcggcggaaagcctgggcctgggcggcgtgtatattggcggcctgcgcaacaacattgaagcggtgaccaaactgctgaaactgccgcagcatgtgctgccgctgtttggcctgtgcctgggctggccggcggataacccggatctgaaaccgcgcctgccggcgagcattctggtgcatgaaaacagctatcagccgctggataaaggcgcgctggcgcagtatgatgaacagctggcggaatattatctgacccgcggcagcaacaaccgccgcgatacctggagcgatcatattcgccgcaccattattaaagaaagccgcccgagcattctggattatctgcataaacagggctgggcgacccgcgctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 18 - DNA, 10239 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctatgaagtgggtgaccttcatcagcctgctgttcctgttcagcagcgcctacagccccaagaagaagatccagctgcacgccgagcacgccctgtacgacgccctgatgatcctgaacatcgtgaagaccaacagcccccccgccgaggagaagctggaggactacgccttcaacttcgagctgatcctggaggagatcgccagactgttcgagagcggcgaccagaaggacgaggccgagaaggccaagagaatgaaggagtggatgaagagaatcaagaccaccgccagcgaggacgagcaggaggagatggccaacgccatcatcaccatcctgcagagctggatcttcagcgctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 19 - DNA, 10392 bp, Artificial Sequence
  Description of Artificial Sequence Synthetic polynucleotide
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctgagcagctcgagagtataatcaactttgaaaaactgactgaatggaccagtggcccgaaaaagtcgacgaatctatctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaaacacatcgcaaccaacgccgttctcttctttggcagatgtgttggatccgagcagctggagtccatctacaggtactacggcctgctgctgaaggaaagatccgaacaaaagcttatttctgaagaggacttggctccagtaaagcagactctaaacttcgatcttctcaagctcgctggagatgttgagagcaacccaggtccagcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaatttaaatgcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgggtaccaattgtgtaatacgactcactatag

SEQ ID NO: 20 - DNA, 20 bp, Artificial Sequence
  Description of Artificial Sequence Synthetic primer
tttgaaatggggggctgggc

SEQ ID NO: 21 - DNA, 19 bp, Artificial Sequence
  Description of Artificial Sequence Synthetic primer
gaggagacccgctaatccg

SEQ ID NO: 22 - DNA, 54 bp, Artificial Sequence
  Description of Artificial Sequence Synthetic primer
tatgggtacctgtaatacgactcactatagggctttgaaatggggggctgggcc

SEQ ID NO: 23 - DNA, 24 bp, Artificial Sequence
  Description of Artificial Sequence Synthetic primer
ccgtcaaagaagcaattctgggca

SEQ ID NO: 24 - DNA, 60 bp, Artificial Sequence
  Description of Artificial Sequence Synthetic primer
gcatgcattttttttttttttttttttttttttttttcccttttctgttccgactgagtt

SEQ ID NO: 25 - DNA, 30 bp, Artificial Sequence
  Description of Artificial Sequence Synthetic primer
ggtaacatgaccttcaattactacgcaaac

SEQ ID NO: 26 - DNA, 23 bp, Artificial Sequence
  Description of Artificial Sequence Synthetic primer
gatcagtacgtcgaaggccgttg

SEQ ID NO: 27 - DNA, 76 bp, Artificial Sequence
  Description of Artificial Sequence Synthetic primer
gcttgcatgcatttaaatttttttttttttttttttttttttttttttcccttttctgttccgactgagttctccc

SEQ ID NO: 28 - DNA, 47 bp, Artificial Sequence
  Description of Artificial Sequence Synthetic primer
caattgtgtaatacgactcactatagtttgaaatggggggctgggcc

SEQ ID NO: 29 - DNA, 765 bp, Homo sapiens
gctgctcacggtcacctgggtgccctggccccagtagtcgcccacgaactccatgccgtactggctgctgcagaagcccacggtggcgcagtagtagtcggcggtgtcctcgggcttcaggctgttcatctgcaggaaggcggtgttcttggcgttgtcccggctgatggtgaaccggcccttcacgaagtcggcgtagtaccgccggccctcgctgccgctgctgatgcagctcacgccctcccgctccttgccgggggcctgccggaaccagccgatggcgtagttgtccaggctgaagccgctggcggcgcagctcagccgcaggctgccgccggggtgcaccaggccgccgccgctctcctgcagctgcacctgaaattcgccgctgcccgccgcgctgcccgcgctgcccaggtgcagctgcaggagagcggcggcggcagcgtgcaggccggcggcagcctgcggctgagctgcaccgccagcggcttcggcgtggacggcaccgacatgggctggtaccggcaggcccccggcaacgagtgcgagctggtgagcagcatcagcagcatcggcatcggctactacagcgagagcgtgaagggccggttcaccatcagccgggacaacgccaagaacaccgtgtacctgcagatgaacagcctgcggcccgacgacaccgccgtgtactactgcggccggcggtggatcggctaccggtgcggcaactggggccggggcacccaggtgaccgtgagcagc

SEQ ID NO: 30 - protein, 255 aa, Homo sapiens
QVQLQESGGGLVHPGGSLRLSCAASGFSLDNYAIGWFRQAPGKEREGVSCISSGSEGRRYYADFVKGRFTISRDNAKNTAFLQMNSLKPEDTADYYCATVGFCSSQYGMEFVGDYWGQGTQVTVSSGSAGSAAGSGEFQVQLQESGGGSVQAGGSLRLSCTASGFGVDGTDMGWYRQAPGNECELVSSISSIGIGYYSESVKGRFTISRDNAKNTVYLQMNSLRPDDTAVYYCGRRWIGYRCGNWGRGTQVTVSS

SEQ ID NO: 31 - DNA, 378 bp, Homo sapiens
ggaggagacggtgacctgggtcccctggccccagtagtcatttgcaccccatcccccttcacccatttttgcacagtaatacacggccgtgtcgtcaggtttcaggctgttcatttgcagatacagcgtgttctcggcgttgtctctggagatggtgaaccggcccttcacggagtctcgatagtatgtgctaccaccattccagctaatatctgagacccactccagccacttccctggagcctgtcggacccagctcatgccataatcatcaaaagtgaatccagaggctgcacaggagagtctcagagaccccccaggctgcaccaagcctcccccagactccaccagctgcacctcctcgagcagctgcacctc

SEQ ID NO: 32 - protein, 126 aa, Homo sapiens
EVQLLEEVQLVESGGGLVQPGGSLRLSCAASGFTFDDYGMSWVRQAPGKWLEWVSDISWNGGSTYYRDSVKGRFTISRDNAENTLYLQMNSLKPDDTAVYYCAKMGEGGWGANDYWGQGTQVTVSS

SEQ ID NO: 33 - DNA, 378 bp, Homo sapiens
gctgctcacggtcacctgggtgccctggccccagtagtcgcccacgaactccatgccgtactggctgctgcagaagcccacggtggcgcagtagtagtcggcggtgtcctcgggcttcaggctgttcatctgcaggaaggcggtgttcttggcgttgtcccggctgatggtgaaccggcccttcacgaagtcggcgtagtaccgccggccctcgctgccgctgctgatgcagctcacgccctcccgctccttgccgggggcctgccggaaccagccgatggcgtagttgtccaggctgaagccgctggcggcgcagctcagccgcaggctgccgccggggtgcaccaggccgccgccgctctcctgcagctgcacctg

SEQ ID NO: 34 - protein, 126 aa, Homo sapiens
QVQLQESGGGLVHPGGSLRLSCAASGFSLDNYAIGWFRQAPGKEREGVSCISSGSEGRRYYADFVKGRFTISRDNAKNTAFLQMNSLKPEDTADYYCATVGFCSSQYGMEFVGDYWGQGTQVTVSS

SEQ ID NO: 35 - DNA, 765 bp, Artificial Sequence
  Description of Artificial Sequence Synthetic polynucleotide
caggtgcagctgcaggagagcggcggcggcctggtgcaccccggcggcagcctgcggctgagctgcgccgccagcggcttcagcctggacaactacgccatcggctggttccggcaggcccccggcaaggagcgggagggcgtgagctgcatcagcagcggcagcgagggccggcggtactacgccgacttcgtgaagggccggttcaccatcagccgggacaacgccaagaacaccgccttcctgcagatgaacagcctgaagcccgaggacaccgccgactactactgcgccaccgtgggcttctgcagcagccagtacggcatggagttcgtgggcgactactggggccagggcacccaggtgaccgtgagcagcggcagcgcgggcagcgcggcgggcagcggcgaatttcaggtgcagctgcaggagagcggcggcggcagcgtgcaggccggcggcagcctgcggctgagctgcaccgccagcggcttcggcgtggacggcaccgacatgggctggtaccggcaggcccccggcaacgagtgcgagctggtgagcagcatcagcagcatcggcatcggctactacagcgagagcgtgaagggccggttcaccatcagccgggacaacgccaagaacaccgtgtacctgcagatgaacagcctgcggcccgacgacaccgccgtgtactactgcggccggcggtggatcggctaccggtgcggcaactggggccggggcacccaggtgaccgtgagcagc

SEQ ID NO: 36 - protein, 255 aa, Artificial Sequence; identical to SEQ ID NO: 30
  Description of Artificial Sequence Synthetic polypeptide

SEQ ID NO: 37 - DNA, 738 bp, Artificial Sequence
  Description of Artificial Sequence Synthetic polynucleotide
caggtgcagctgcaggagagcggcggcggcagcgtgcaggccggcggcagcctgcggctgagctgcaccgccagcggcttcggcgtggacggcaccgacatgggctggtaccggcaggcccccggcaacgagtgcgagctggtgagcagcatcagcagcatcggcatcggctactacagcgagagcgtgaagggccggttcaccatcagccgggacaacgccaagaacaccgtgtacctgcagatgaacagcctgcggcccgacgacaccgccgtgtactactgcggccggcggtggatcggctaccggtgcggcaactggggccggggcacccaggtgaccgtgagcagcggcagcgcgggcagcgcggcgggcagcggcgaatttcaggtgcagctgcaggagagcggcggcggcagcgtgcaggccggcggcagcctgcggctgagctgcaccgccagcggcttcggcgtggacggcaccgacatgggctggtaccggcaggcccccggcaacgagtgcgagctggtgagcagcatcagcagcatcggcatcggctactacagcgagagcgtgaagggccggttcaccatcagccgggacaacgccaagaacaccgtgtacctgcagatgaacagcctgcggcccgacgacaccgccgtgtactactgcggccggcggtggatcggctaccggtgcggcaactggggccggggcacccaggtgaccgtgagcagc

SEQ ID NO: 38 - protein, 255 aa, Artificial Sequence
  Description of Artificial Sequence Synthetic polypeptide
EVQLLEEVQLVESGGGLVQPGGSLRLSCAASGFTFDDYGMSWVRQAPGKWLEWVSDISWNGGSTYYRDSVKGRFTISRDNAENTLYLQMNSLKPDDTAVYYCAKMGEGGWGANDYWGQGTQVTVSSGSAGSAAGSGEFQVQLQESGGGSVQAGGSLRLSCTASGFGVDGTDMGWYRQAPGNECELVSSISSIGIGYYSESVKGRFTISRDNAKNTVYLQMNSLRPDDTAVYYCGRRWIGYRCGNWGRGTQVTVSS

SEQ ID NO: 39 - DNA, 792 bp, Artificial Sequence
  Description of Artificial Sequence Synthetic polynucleotide
caggtgcagctgcaggagagcggcggcggcctggtgcaccccggcggcagcctgcggctgagctgcgccgccagcggcttcagcctggacaactacgccatcggctggttccggcaggcccccggcaaggagcgggagggcgtgagctgcatcagcagcggcagcgagggccggcggtactacgccgacttcgtgaagggccggttcaccatcagccgggacaacgccaagaacaccgccttcctgcagatgaacagcctgaagcccgaggacaccgccgactactactgcgccaccgtgggcttctgcagcagccagtacggcatggagttcgtgggcgactactggggccagggcacccaggtgaccgtgagcagcggcagcgcgggcagcgcggcgggcagcggcgaatttgaggtgcagctgctcgaggaggtgcagctggtggagtctgggggaggcttggtgcagcctggggggtctctgagactctcctgtgcagcctctggattcacttttgatgattatggcatgagctgggtccgacaggctccagggaagtggctggagtgggtctcagatattagctggaatggtggtagcacatactatcgagactccgtgaagggccggttcaccatctccagagacaacgccgagaacacgctgtatctgcaaatgaacagcctgaaacctgacgacacggccgtgtattactgtgcaaaaatgggtgaagggggatggggtgcaaatgactactggggccaggggacccaggtcaccgtctcctcc

SEQ ID NO: 40 - protein, 264 aa, Artificial Sequence
  Description of Artificial Sequence Synthetic polypeptide
QVQLQESGGGLVHPGGSLRLSCAASGFSLDNYAIGWFRQAPGKEREGVSCISSGSEGRRYYADFVKGRFTISRDNAKNTAFLQMNSLKPEDTADYYCATVGFCSSQYGMEFVGDYWGQGTQVTVSSGSAGSAAGSGEFEVQLLEEVQLVESGGGLVQPGGSLRLSCAASGFTFDDYGMSWVRQAPGKWLEWVSDISWNGGSTYYRDSVKGRFTISRDNAENTLYLQMNSLKPDDTAVYYCAKMGEGGWGANDYWGQGTQVTVSS

SEQ ID NO: 41 - DNA, 399 bp, Homo sapiens
gcgccgaccagcagcagcaccaaaaaaacccagctgcagctggaacatctgctgctggatctgcagatgattctgaacggcattaacaactataaaaacccgaaactgacccgcatgctgacctttaaattttatatgccgaaaaaagcgaccgaactgaaacatctgcagtgcctggaagaagaactgaaaccgctggaagaagtgctgaacctggcgcagagcaaaaactttcatctgcgcccgcgcgatctgattagcaacattaacgtgattgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 42 - protein, 133 aa, Homo sapiens [sequence omitted]

SEQ ID NO: 43 - DNA, 459 bp, Homo sapiens
atgtatcgcatgcagctgctgagctgcattgcgctgagcctggcgctggtgaccaacagcgcgccgaccagcagcagcaccaaaaaaacccagctgcagctggaacatctgctgctggatctgcagatgattctgaacggcattaacaactataaaaacccgaaactgacccgcatgctgacctttaaattttatatgccgaaaaaagcgaccgaactgaaacatctgcagtgcctggaagaagaactgaaaccgctggaagaagtgctgaacctggcgcagagcaaaaactttcatctgcgcccgcgcgatctgattagcaacattaacgtgattgtgctggaactgaaaggcagcgaaaccacctttatgtgcgaatatgcggatgaaaccgcgaccattgtggaatttctgaaccgctggattaccttttgccagagcattattagcaccctgacc

SEQ ID NO: 44 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 45 - DNA, 534 bp, Homo sapiens
acgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtttacccatacgatgttccagattacgct

SEQ ID NO: 46 - protein, 178 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVYPYDVPDYA

SEQ ID NO: 47 - DNA, 597 bp, Homo sapiens
ggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtttacccatacgatgttccagattacgct

SEQ ID NO: 48 - protein, 199 aa, Homo sapiens
GRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVYPYDVPDYA

SEQ ID NO: 49 - DNA, 474 bp, Homo sapiens
atggtgaccggcggcatggccagcaagtgggaccagaagggcatggacatcgcctacgaggaggccctgctgggctacaaggagggcggcgtgcccatcggcggctgcctgatcaacaacaaggacggcagcgtgctgggcagaggccacaacatgagattccagaagggcagcgccaccctgcacggcgagatcagcaccctggagaactgcggcagactggagggcaaggtgtacaaggacaccaccctgtacaccaccctgagcccctgcgacatgtgcaccggcgccatcatcatgtacggcatccccagatgcgtgatcggcgagaacgtgaacttcaagagcaagggcgagaagtacctgcagaccagaggccacgaggtggtggtggtggacgacgagagatgcaagaagctgatgaagcagttcatcgacgagagaccccaggactggttcgaggacatcggcgag

SEQ ID NO: 50 - protein, 158 aa, Homo sapiens
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGE

SEQ ID NO: 51 - DNA, 717 bp, Homo sapiens
atgaccccgaccattgaactgatttgcggccatcgcagcattcgccattttaccgatgaaccgattagcgaagcgcagcgcgaagcgattattaacagcgcgcgcgcgaccagcagcagctattttctgcagtgcagcagcattattcgcattaccgataaagcgctgcgcgaagaactggtgaccctgaccggcggccagaaacatgtggcgcaggcggcggaattttgggtgttttgcgcggattttaaccgccatctgcagatttgcccggatgcgcagctgggcctggcggaacagctgctgatgggctgggataccgcgatgatggcgcagaacgcgctgattgcggcggaaagcctgggcctgggcggcgtgtatattggcggcctgcgcaacaacattgaagcggtgaccaaactgctgaaactgccgcagcatgtgctgccgctgtttggcctgtgcctgggctggccggcggataacccggatctgaaaccgcgcctgccggcgagcattctggtgcatgaaaacagctatcagccgctggataaaggcgcgctggcgcagtatgatgaacagctggcggaatattatctgacccgcggcagcaacaaccgccgcgatacctggagcgatcatattcgccgcaccattattgaagaaagcgcgccgtttattctggattatctgcataaacagggctgggcgacccgc

SEQ ID NO: 52 - protein, 239 aa, Homo sapiens
MTPTIELICGHRSIRHFTDEPISEAQREAIINSARATSSSYFLQCSSIIRITDKALREELVTLTGGQKHVAQAAEFWVFCADFNRHLQICPDAQLGLAEQLLMGWDTAMMAQNALIAAESLGLGGVYIGGLRNNIEAVTKLLKLPQHVLPLFGLCLGWPADNPDLKPRLPASILVHENSYQPLDKGALAQYDEQLAEYYLTRGSNNRRDTWSDHIRRTIIEESAPFILDYLHKQGWATR